(19) United States
(12) Patent Application Publication
(10) Pub. No.: US 2003/0158672 A1
(43) Pub. Date: Aug. 21, 2003

(54) Use of computationally derived protein structures of genetic polymorphisms in pharmacogenomics for drug design and clinical applications

(76) Inventors: Kalyanaraman Ramnarayan, San Diego, CA (US); Edward T. Maggio, San Diego, CA (US); P. Patrick Hess, San Clemente, CA (US)

Correspondence Address: Stephanie Seidman, Heller Ehrman White & McAuliffe LLP, 7th Floor, 4350 La Jolla Village Drive, San Diego, CA, 92122-1246, US

(21) Appl. No.: 10/271,181

(22) Filed: Oct. 10, 2002

Related U.S. Application Data
(62) Division of application No. 09/709,905, filed on Nov. 10, 2000.
Application No. 09/709,905 is a continuation-in-part of application No. 09/438,566, filed on Nov. 10, 1999, now abandoned.
Application No. 09/709,905 is a continuation-in-part of application No. 09/704,362, filed on Nov. 1, 2000.
Application No. 09/704,362 is a continuation of application No. 09/438,566, filed on Nov. 10, 1999, now abandoned.

(51) Int. Cl. G06G 7/48; G06G 7/58; G06F 19/00; G01N 33/48; G01N 33/50

(52) U.S. Cl. 702/19; 703/11; 702/20

(57) ABSTRACT

Provided herein are computer-based methods for generating and using three-dimensional (3-D) structural models of target molecules and databases containing the models. The targets can be protein structural variants derived from genes containing polymorphisms. The models are generated using molecular modeling techniques and are used in structure-based drug design studies for identifying drugs that bind to particular structural variants in structure-based drug design studies, for designing allele-specific drugs and population-specific drugs and for predicting clinical responses in patients. Computer-based methods for predicting drug resistance or sensitivity via computational phenotyping are also provided. Databases containing protein structural variant models are also provided.

### RELATED APPLICATIONS

This application is a divisional of U.S. application Ser. No. 09/709,905 to Kalyanaraman Ramnarayan, Edward T. Maggio and P. Patrick Hess, filed Nov. 10, 2000 entitled “USE OF COMPUTATIONALLY DERIVED PROTEIN STRUCTURES OF GENETIC POLYMORPHISMS IN PHARMACOGENOMICS FOR DRUG DESIGN AND CLINICAL APPLICATIONS.” U.S. application Ser. No. 09/709,905 is a continuation-in-part of U.S. application Ser. No. 09/438,566 to Kalyanaraman Ramnarayan, Edward T. Maggio and P. Patrick Hess, filed Nov. 10, 1999 entitled “USE OF COMPUTATIONALLY DERIVED PROTEIN STRUCTURES OF GENETIC POLYMORPHISMS IN PHARMACOGENOMICS FOR DRUG DESIGN AND CLINICAL APPLICATIONS”; and U.S. application Ser. No. 09/704,362 to Kalyanaraman Ramnarayan, Edward T. Maggio and P. Patrick Hess, filed Nov. 1, 2000, entitled “USE OF COMPUTATIONALLY DERIVED PROTEIN STRUCTURES OF GENETIC POLYMORPHISMS IN PHARMACOGENOMICS FOR DRUG DESIGN AND CLINICAL APPLICATIONS.” U.S. application Ser. No. 09/704,362 is a continuation of U.S. application Ser. No. 09/438,566. Priority is claimed herein to the above-noted applications, the disclosures of which are incorporated by reference herein in their entirety.

### INCORPORATION BY REFERENCE OF TABLES PROVIDED ON COMPACT DISKS

An electronic version on compact disk (CD) ROM of Tables 4 and 5, which set forth coordinates for three-dimensional structures of proteins in the database described herein is filed herewith. The contents thereof is incorporated by reference in its entirety. Table 4 is the HIV reverse transcriptase coordinates, and Table 5 is the HIV protease coordinates. The files that contain Table 4 are entitled 1906DTAB.001 and 1906DTAB.002, created on Oct. 9, 2002, and are 59,538 kilobytes and 304 kilobytes, respectively. The file that contains Table 5 is entitled 1906DTAB.003, created on Oct. 9, 2002, and contains 11,413 kilobytes.

### FIELD OF THE INVENTION

The present invention is related to computer-based methods and relational databases that use three-dimensional (3-D) protein structural models derived from genetic polymorphisms in the areas of computer-assisted drug design and the prediction of clinical responses in patients.

### BACKGROUND OF THE INVENTION

Recent advances in molecular biology, such as the discovery and identification of large numbers of genes and the sequences thereof encoded in the genomes of humans, other mammals and infectious disease agents, have contributed to the identification of a large number of proteins, biological receptors and other macromolecules and complexes that are promising therapeutic targets. Based on the information derived from the gene sequences, the three-dimensional (3-D) molecular structures of the corresponding target proteins or receptors can be determined.

Since 3-D protein structure is related to biological function, structure-based drug design is an increasingly useful methodology that has made a great impact in the design of biologically active lead compounds. Drug designers can design and screen potential new drugs via computational methods, such as docking or binding studies, before actually beginning patient testing. These experiments can be performed in silico at a tiny fraction of the clinical cost.

The resulting molecules, while serving as lead compounds, often have unpredictable effects when employed in clinical trials. In addition, it has been observed that existing drugs with known clinical efficacy far often fail to achieve beneficial results when given to particular patients, or particular subpopulations, such as ethnic groups, of patients. Genetic stratification of a population can be the difference between drug failure and drug approval. Hence there is a need to develop methods to improve the drug discovery process. Therefore, it is an object herein to provide, among a variety of benefits, methods and products that address and solve these problems. In particular, it is an object herein to provide computationally-based methods for drug design, clinical testing protocols, identification of new drug candidates and drug therapies; for predicting drug sensitivity and resistance and other methods.

### SUMMARY OF THE INVENTION

Provided herein are computer-based methods for generating and using three-dimensional (3-D) structural models of target biomolecules, particularly polymorphic and allelic variants. Also provided herein are databases that contain the sequences of such variants and also the 3-D structure of the variants for use with the methods.

Genetic polymorphisms arise, for example, as a result of gene sequence differences or as a result of post-translational modifications, including glycosylation. Hence genetic polymorphisms are manifested as gene products and proteins having variant structures. The variant structures result in differences in biological responses among the originating organisms. These differences in response, include, but are not limited to, differences among patient responses to a particular drug, effective dosage differences, and side effects. With respect to infectious organisms, some polymorphisms may arise that convey resistance or susceptibility to particular drug therapies by the altering the drug target structure.

Structural changes that arise as a result of genetic polymorphisms are not of unlimited variety, since 3-D structure impacts upon function. A knowledge of the repertoire of the fine differences among generally similar 3-D structures of particular proteins will permit design of drugs that bind to the most polymorphisms, drugs that induce the fewest side-effects, and drugs that are more effective against infectious agents. Knowledge of these structures ultimately will permit patient-specific or subpopulation-specific, such as ethnic, age, or gender groups, design or selection of drugs.

The methods that are provided are for determining and using 3-dimensional (3-D) protein structures that are derived from genetic polymorphisms to understand differences in biological activity that result from the polymorphisms, and to use this understanding to aid in the identification of potential new drug candidates and drug therapies. Also provided are methods for analyzing 3-D structures of protein structural variant targets derived from genetic polymorphisms to identify common structural features among the variants; methods for identifying structural changes in target proteins that are associated with multiple mutations arising from genetic polymorphisms and correlating this information with biological activity; and methods for using clinical data in conjunction with structural variants derived from genetic polymorphisms to understand and predict the pharmacological effects and clinical outcomes for drugs or potential drugs. Also provided are methods for generating 3-D protein structures derived from a given genotype to analyze protein-drug binding in silico to predict drug sensitivity or resistance. Also provided are databases that are used in methods provided herein and methods for generating the databases.

In particular, target biomolecules are protein structural variants encoded by genes containing genetic variations, or polymorphisms. 3-D models of the structures of proteins are determined. The models are generated using molecular modeling techniques, such as homology modeling. The resulting models are then used in the methods provided herein, which include structure-based drug design studies to design and identify drugs that bind to particular structural variants; structure-based drug design studies and to predict clinical responses in patients; and to design drugs that bind to all or a substantial portion of allelic variants of a target, to thereby increase the population of patients for whom a particular drug will be effective and/or to decrease the undesirable side-effects in a larger population.

Hence, computer-based methods of drug design based on target protein structural models derived from genetic polymorphisms are provided. The methods involve obtaining one, preferably two or more amino acid sequences of a target protein that is the product of a gene exhibiting genetic polymorphisms, where sequences represent different genetic polymorphisms, and generating 3-D protein structural variant models from the sequences. Structure-based drug design techniques are used to design potential new drug candidates or to suggest modifications to existing drugs based on predicted intermolecular interactions of the drugs or drug candidates with the models. Alternatively, drug molecules can be computationally docked with 3-D protein structural variant models based upon the sequences and energetically refined before performing structure-based drug design studies.

In preferred embodiments, binding interactions between a drug or potential new drug candidate molecule and the structural variants are calculated in order to optimize intermolecular interactions between drug or potential drug molecules and the structural variant models or to select drug therapies for patients by determining a drug or drugs that have favorable binding interactions with the structural variant models.

In other embodiments, the binding interactions are determined by calculating the free energy of binding between the protein structural variant model and a docked molecule; and decomposing the total free energy of binding based on the interacting residues in the protein active site.

After the protein structural variant models are generated, selected model structures are analyzed to determine common structural features that are conserved throughout the selected models. The conserved structural features can serve as scaffolds or pharmacophore models into which potential drugs or modified drugs are docked. For example, the selected model structures may represent the structural variants resulting from the most commonly occurring genetic polymorphisms or from genetic polymorphisms found in a specific patient subpopulation, such as a particular age group, ethnic or racial group, sex, or other subpopulation. Alternatively, the models may be selected based on clinical information, for example, the structural variants may be derived based on patients receiving a specific treatment regimen or exhibiting a particular clinical response to a given drug or on the duration of a particular drug treatment.

The methods provided herein can be used for predicting clinical responses in patients based on genetic polymorphisms. For example, a structural variant model derived from a subject, such as a human patient, exhibiting a particular genetic polymorphism is generated and screened against a number of reference protein structural variant models derived from genetic polymorphisms of the same gene in other such subjects. In certain embodiments, the reference structures are stored in a database, preferably with observed clinical data associated with the structures, or polymorphisms. The structural variant model from the subject is compared to a reference structures, for example, by database searching, in order to identify reference structural variants that are similar to the model structure derived from the subject. Based on the premise that structurally similar targets will have similar clinical responses, a clinical outcome can be predicted for the patient based on the structures identified through structural comparison or database searching. This information can also be used in the design and analysis of clinical trials; it can also be used for selecting appropriate therapies for a subject in instances in which the subject is a patient and the protein is a drug target.

The methods are also used to design therapeutic agents that are active against biological targets that have become drug resistant, particularly due to genetic mutations. In certain embodiments, 3-D protein structural variant models are generated for a target protein in which genetic mutations have occurred and against which a given drug is no longer biologically active. The models are compared to 3-D protein structural variant models of the target protein against which the drug has biological activity in order to identify structural differences between the susceptible and resistant targets. The differences can be used to understand the structural contributions to drug resistance, and this information can be utilized in structure-based drug design calculations to identify new drugs or modifications to the existing drug that circumvent the resistance problem.

A computer-based method for identifying compensatory mutations in a target protein is also provided. The method involves obtaining the amino acid sequence of a target protein containing multiple amino acid mutations that is expressed in a patient, where the structure of a form of the target protein that responds to a particular drug, including the active site, has been structurally characterized; generating a 3-D structural model of the mutated protein; comparing the structure of the mutated protein with the form of the protein that responds to the drug to identify structural differences and/or similarities arising from the mutations; comparing the biological activities of the drug against the mutated protein and the form of the protein that responds to the drug to determine the effects of the mutations on drug response; and identifying the mutations in the protein that affect biological activity based on the comparisons. The target biolmolecules can also be used in a method referred to herein as computational phenotyping to predict drug sensitivity or resistance for a given genotype. These computer-based method for identifying phenotypes in silico are provided. The methods involve obtaining from a patient/specimen, such as a body fluid or tissue sample, including blood, cerebral spinal fluid, urine, saliva, sweat and tissue samples, the amino acid sequence of a target protein; generating a 3-D structural model of the target protein; performing protein-drug binding analyses; and predicting drug sensitivity or resistance based on the protein-drug binding analyses.

Molecular structure databases containing protein structural variant models produced by the methods are also provided. The databases may also contain biological or clinical data associated with the structural variants. The databases can be interfaced to a molecular graphics package for visualization and analysis of the 3-D molecular structural models. In particular, databases containing the 3-D structures of polymorphic variants of selected target genes, particularly pharmaceutically significant genes with pharmaceutically significant gene products, such as proteases and polymerases, including reverse transcriptases, and receptors, such as cell surface receptors, are provided. The databases may be stored an provided on any suitable medium, including, but are not limited to, floppy disks, hard drives, CD-ROMS and DVDs.

Also provided are relational databases for managing and using information relating to genetic polymorphisms. The databases contain 3-D molecular coordinates for structural variants derived from genetic polymorphism, a molecular graphics interface for 3-D molecular structure visualization, computer functionality for protein sequence and structural analyses and database searching tools. The databases may further include observed clinical data associated with the genetic polymorphism. The databases provide a means to design the allele-specific drugs and also to identify among alleles common or conserved structural features that can serve as the target for drug design.

The databases can also be used for identification of invariant residues and regions of a target biomoleucle, such as an HIV protease or reverse transcriptase. The identified invariant regions are then used to computationally screen compounds, preferably small molecules by assessing binding interactions. The compounds so-identified serve as candidates for drugs that will be effective for a larger proporation of a population or against a broader range of variants of a pathogen, where the target protein is from a pathogens.

Systems, including computers, containing the databases also are provided herein. Any computer known to those of skill in the art for maintaining such databases is contemplated. User interfaces for accessing and manipulating the databases and content thereof are also provided.

### BRIEF DESCRIPTION OF THE DRAWINGS

FIG. 1 illustrates a method for creating a protein structural variant relational database.

FIG. 2 is a flow chart that describes one method used to generate structural variant models derived from genetic polymorphisms and to use the models in structure-based drug design studies.

FIG. 3 is a flow chart that describes an alternative method used to generate structural variant models derived from genetic polymorphisms and to use the models in structure-based drug design studies.

FIG. 4 shows the correlation between experimental and calculated changes of binding energy upon ligand modifications in the binding site of NS3.

FIG. 5 shows a comparison of calculated versus experimental binding free energy changes for complexes of the tumor necrosis factor (TNF) receptor with different inhibitors.

FIG. 6 shows the HIV PR inhibitors approved by the FDA.

FIG. 7 shows the frequency versus amino acid residue plot of HIV PR.

FIG. 8 shows frequency analysis of 10591 HIV PR Sequences, where ResNum is the residue number; TotOcc is the total occurrence of the mutation; Dist is the distance of the mutating residue from approximate center of active site (Asp28); WtAA is the amino acid in the wild type protein; NumMut is the number of mutations; and MutList is a list of amino acid mutations.

FIG. 9 is a block diagram of an exemplary computer.

FIG. 10 is a graphical representation of a relational database.

FIG. 11 is a tabulation of the 3-D coordinates of a representative entry in a database that includes 3-D structures.

### DETAILED DESCRIPTION OF THE INVENTION

A. Definitions

B. Computer-based methods of drug design based on genetic polymorphisms

1. Methods for obtaining amino acid sequences of a target protein

2. Generation of 3-D protein structural variant models

a. Homology Modeling

b. Ab initio generation of 3-D structures

c. Crystal structures

3. Use of 3-D structural variant models in drug design

a. Selection of relevant structural variants

b. Drug design

c. Computational docking

d. Free energy of binding studies

C. Applications of computer-based methods

1. Genetic polymorphisms and structure-based drug design

2. Drug resistance

3. Identification of conserved structural features or pharmacophores

4. Identification of compensatory structural changes

5. Clinical Applications

D. Creation of 3-D Structural Polymorphism Databases

1. Exemplary Databases and generation thereof

2. Computer systems and Database

E. Computational phenotyping

### A. Definitions

Unless defined otherwise, all technical and scientific terms used herein have the same meaning as is commonly understood by one of skill in the art to which this invention belongs. All patents, patent applications, published patent applications and publications referred to herein are, unless noted otherwise, incorporated by reference in their entirety. In the event a definition in this section is not consistent with definitions elsewhere, the definition set forth in this section will control.

As used herein, polymorphism refers to a variation in the sequence of a gene in the genome amongst a population, such as allelic variations and other variations that arise or are observed. Genetic polymorphisms refers to the variant forms of gene sequences that can arise as a result of nucleotide base pair differences, alternative mRNA splicing or post-translational modifications, including, for example, glycosylation. Thus, a polymorphism refers to the occurrence of two or more genetically determined alternative sequences or alleles in a population. These differences can occur in coding and non-coding portions of the genome, and can be manifested or detected as differences in nucleic acid sequences, gene expression, including, for example transcription, processing, translation, transport, protein processing, trafficking, DNA synthesis, expressed proteins, other gene products or products of biochemical pathways or in post-translational modifications and any other differences manifested among members of a population. A single nucleotide polymorphism (SNP) refers to a polymorphism that arises as the result of a single base change, such as an insertion, deletion or change in a base.

A polymorphic marker or site is the locus at which divergence occurs. Such site may be as small as one base pair (an SNP). Polymorphic markers include, but are not limited to, restriction fragment length polymorphisms, variable number of tandem repeats (VNTR's), hypervariable regions, minisatellites, dinucleotide repeats, trinucleotide repeats, tetranucleotide repeats and other repeating patterns, simple sequence repeats and insertional elements, such as Alu. Polymorphic forms also are manifested as different mendelian alleles for a gene. Polymorphisms may be observed by differences in proteins, protein modifications, RNA expression modification, DNA and RNA methylation, regulatory factors that alter gene expression and DNA replication, and any other manifestation of alterations in genomic nucleic acid or organelle nucleic acids.

As used herein, structural variant proteins refer the variety of 3-D molecular structures or models thereof that result from the polymorphisms. These variants typically arise from transcription and translation of genes containing genetic polymorphisms, but also include diffentially glyosylated or otherwise post-translationally modified variants that potentially exhibit differential interactions with drugs and drug candidates.

As used herein, binding interactions refer to atomic or physical interactions between molecules including, but not limited to binding free energy, hydrophobic interactions, electrostatic interactions, steric interactions and other interactions that are commonly considered by those of skill in the art to determine the affinity of one molecule to bind to another. Favorable binding interactions refer to binding interactions that promote physical or chemical associations between molecules.

As used herein, a target protein is defined as a protein that is a receptor with which drugs or other ligands, such as small molecule or peptide agonists or antagonists or other proteins or biomacromolecules, such as DNA or RNA, interact to bring about a biological response.

As used herein, structure-based drug design refers to computer-based methods in which 3-D coordinates for molecular structures are used to identify potential drugs that can interact with a biological receptor. Examples of such methods include, but are not limited to, searching of small molecule libraries or databases, conformational searching of a ligand within an active site of identify biologically active conformations or computational docking methods.

As used herein, pharmacogenomics refers to study of the variability of patient responses to drugs due to inherent genetic differences.

As used herein, computational docking refers to techniques wherein molecules, for example, a ligand and receptor or active site, are fitted together based on complementary interactions, for example, steric, hydrophobic or electrostatic interactions.

As used herein, energetic refinement refers to the use of molecular mechanics simulation techniques, such as energy minimization or molecular dynamics, or other techniques, such as quantum-based approaches, to “adjust” the coordinates of a molecular structural model to bring it into a stable, low energy, conformation. In molecular mechanics simulations, the potential energy of a molecular system is represented as a function of its atomic coordinates along with a set of atomic parameters, called a forcefield. Energy minimization refers to a method wherein the coordinates of a molecular conformation are adjusted according to a target function to result in a lower energy conformation. Molecular dynamics refers to methods for simulating molecular motion by inputting kinetic energy into the molecular system corresponding to a specified temperature, and integrating the classical equations of motion for the molecular system. During a molecular dynamics simulation, a system undergoes conformational changes so that different parts of its accessible phase space are explored.

As used herein, clinical data refers to information obtained from patients pertaining to pharmacological responses of the patient to a given drug, including, but not limited to efficacy data, side effects, resistance or susceptibility to drug therapy, pharmacokinetics or clinical trial results.

As used herein, patient histories, include medical histories and other any information, such as parental medical histories, dates and places of birth of the patient and parents, number of siblings, number of children and other such data.

As used herein, compensatory mutations are mutations that act in concert with active site mutations by compensating for functional deficits caused by changes or mutations that affect binding in the active site.

As used herein, a relational database is a collection of data items organized as a set of formally-described tables from which data can be accessed or reassembled in many different ways without having to reorganize the database tables. Such databases are readily available commercially, for example, from Oracle, IBM, Microsoft, Sybase, Computer Associates, SAP, or multiple other vendors.

As used herein, a phenotype refers to a set of parameters that includes any distinguishable trait of an organism. A phenotype can be physical traits and can be, in instances in which the subject is an animal, a mental trait, such as emotional traits. Some phenotypes can be determined by observation elicited by questionnaires or by referring to prior medical and other records. For purposes herein, a phenotype is a parameter around which the database can be sorted.

As used herein, genotype refers to a specific gene or totality of genetic information in a specific cell or organism.

As used herein, haplotype refers refers to two or more polymorphism located on a single DNA strand. Hence, haplotyping refers to identification of two or more polymorphisms on a single DNA strand. Haplotypes can be indicative of a phenotype.

As used herein, a parameter is any input data that will serve as a basis for sorting the database. These parameters will include phenotypic traits, medical histories, family histories and any other such information elicited from a subject or observed about the subject. A parameter may describe the subject, some historical or current environmental or social influence experienced by the subject, or a condition or environmental influence on someone related to the subject. Paramaters include, but are not limited to, any of those described herein, and known to those of skill in the art.

As used herein, computational phenotyping, refers to computer-based processes that assess the phenotype resulting from a particular genotype. The phenotype describes observables, such as, but are not limited to, the structure of the encoded protein, its functional morphological and structural attributes. In particular, as contemplated herein, the phenotype that is assesed is the interaction of a protein with a particular compounds, particularly a drug. As exemplified herein, the method provides a means to select an effective drug for a particular subjects, particularly mammals, or class thereof.

As used herein, a database refers to a collection of data; in this case data relating to polymorphic variants. Hence a database contains the nucleic acid sequences encoding the variants, or a portion of the variant, such as a portion contianing the active site or targeted site. Additionally, the database may contain other information related to each entry, including but are not limited to, the corresponding 3-D structure of the encoded protein (or a portion thereof) and information regarding the source of each sequence. Some of the entries in a database may be identical, and for purposes herein, a database contains at least 2 different entries, typically far more than 2 entries. The number of entries depends upon the protein of interest and variety and number of polymorphisms that exist. Generally a database will have at least 10 different entries, typically more than 100, more than 500, more than 1000, more than 2000, 3000, 4000, 5000, 8000, 10,000, 50,000, 100,000 and greater. Databases herein containing 20,000 entries and more have been generated and are exemplified herein.

As used herein, a relational database stores information in a form representative of matrices, such as two-dimensional tables, including rows and columns of data, or higher dimensional matrices. For example, in one embodiment, the relational database has separate tables each with a parameter. The tables are linked with a record number, which also acts as an index. The database can be searched or sorted by using data in the tables and is stored in any suitable storage medium, such as floppy disk, CD rom disk, hard drive or other suitable medium.

As used herein, a profile refers to information relating to, but not limited to and not necessarily including all of, age, sex, ethnicity, disease history, family history, phenotypic characteristics, such as height and weight and other relevant parameters.

As used herein, a biopolymer includes, but is not limited to, nucleic acid, proteins, polysaccharides, lipids and other macromolecules. Nucleic acids include DNA, RNA, and fragments thereof. Nucleic acids may be derived from genomic DNA, RNA, mitochondrial nucleic acid, chloroplast nucleic acid and other organelles with separate genetic material.

As used herein, a DNA or nucleic acid homolog refers to a nucleic acid that includes a preselected conserved nucleotide sequence. By the term “substantially homologous” is meant having at least 80%, preferably at least 90%, most preferably at least 95% homology therewith or a less percentage of homology or identity and conserved biological activity or function.

As used herein, a receptor refers to a molecule that has an affinity for a given ligand. Receptors may be naturally-occurring or synthetic molecules. Receptors may also be referred to in the art as anti-ligands. As used herein, the terms, receptor and anti-ligand are interchangeable. Receptors can be used in their unaltered state or as aggregates with other species. Receptors may be attached, covalently or noncovalently, or in physical contact with, to a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants (such as on viruses, cells, or other materials), drugs, polynucleotides, nucleic acids, peptides, cofactors, lectins, sugars, polysaccharides, cells, cellular membranes, and organelles.

Examples of receptors and applications using such receptors, include but are not restricted to:

a) enzymes: specific transport proteins or enzymes essential to survival of microorganisms, which could serve as targets for antibiotic (ligand) selection;

b) antibodies: identification of a ligand-binding site on the antibody molecule that combines with the epitope of an antigen of interest may be investigated; determination of a sequence that mimics an antigenic epitope may lead to the development of vaccines of which the immunogen is based on one or more of such sequences or lead to the development of related diagnostic agents or compounds useful in therapeutic treatments such as for auto-immune diseases;

c) nucleic acids: identification of ligand, such as protein or RNA, binding sites;

d) catalytic polypeptides: polymers, preferably polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant (see, e.g., U.S. Pat. No. 5,215,899);

e) hormone receptors: determination of the ligands that bind with high affinity to a receptor is useful in the development of hormone replacement therapies; for example, identification of ligands that bind to such receptors may lead to the development of drugs to control blood pressure; and

f) opiate receptors: determination of ligands that bind to the opiate receptors in the brain is useful in the development of less-addictive replacements for morphine and related drugs.

As used herein, prion refers to an infectious pathogen that causes central nervous system spongiform encephalopathies in humans and animals. No nucleic acid component is necessary for the infectivity of prion protein (see, e.g., U.S. Pat. No. 5,808,969).

As used herein, a ligand is a molecule that is specifically recognized by a particular receptor. Examples of ligands, include, but are not limited to, agonists and antagonists for cell membrane receptors, toxins and venoms, viral epitopes, hormones (e.g., steroids), hormone receptors, opiates, peptides, enzymes, enzyme substrates, cofactors, drugs, lectins, sugars, oligonucleotides, nucleic acids, oligosaccharides, proteins, and monoclonal antibodies.

As used herein, complementary refers to the topological compatibility or matching together of interacting surfaces of a ligand molecule and its receptor. Thus, the receptor and its ligand can be described as complementary, and furthermore, the contact surface characteristics are complementary to each other.

As used herein, a ligand-receptor pair or complex formed when two macromolecules have combined through molecular recognition to form a complex.

The terms “homology” and “identity” are often used interchangeably. In this regard, percent homology or identity may be determined, for example, by comparing sequence information using a GAP computer program. The GAP program utilizes the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine identity In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipman, D., SIAM J Applied Math 48:1073 (1988)). Methods commonly employed to determine identity or similarity between two sequences include, but are not limited to, those disclosed in Guide to Human Genome Computing, Martin J. Bishop, ed., Academic Press, San Diego, 1998, and Carillo, H. & Lipman, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide. For example, a test polypeptide may be defined as any polypeptide that is 90% or more identical to a reference polypeptide.

As used herein, the term at least “90% identical to” refers to percent identities from 90 to 99.99 relative to a reference polypeptide. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). Differences are defined as nucleic acid or amino acid substitutions, or deletions.

As used herein, AMBER is a force field well known in the arts and designed for the study of proteins and nucleic acids as defined in Weiner et al. J. Comput. Chem. (1986) 7:230-252, where a modified AMBER (version 3.3) force field is a fully vectorized version of AMBER (version 3.0) with coordinate coupling, intra/inter decomposition, and the option to include the polarization energy as part of the total energy. AMBER is available in commercially available molecular modeling programs such as, but not limited to, Macromodel (Columbia University).

As used herein, ECEPP (Empirical Conformational Energies of Peptides Program) is a force field well know in the arts (U.S. Pat. Nos. 5,910,478; 5,846,763). ECEPP/3 refers to version 3 of this well known force field.

As used herein, QSAR refers to quantitative structure-activity relationship.

As used herein, vdw refers to van der Waals.

As used herein, RMSD refers to root mean-squared deviation.

As used herein, medical history refers to the parameters and data typically obtained by a physician when examining a subject or other such professional when examining other mammals, and includes such information as prior diseases, age, weight, height, sex and other information. For purposes, the subjects that serve as the source of the samples from which nucleic acids encoding polymorphisms are isolated, include animals, plants, pathogens and any organism that has nucleic acid that exhibits polymorphism. In this context medical history refers to information pertinent to the particular organism.

As used herein, subject history, refers to data such as locale in which the subject was born, raised or resident or visited, and parental history and other such information.

As used herein, a drug is an agent that binds to or interacts with a targeted protein. For purposes, a therapeutic agent is a drug.

### B. Computer-Based Methods of Drug Design Based on Genetic Polymorphisms

Methods for computer-based drug design based on genetic poly-morphisms are provided. The methods includes the steps of obtaining one or more, preferably two or more, amino acid sequences of a target protein that is the product of a gene exhibiting genetic polymorphisms; generating 3-dimensional (3-D) protein structural variant models of all or a portion of the protein from the sequences; and based upon the structures of the 3-D models, designing drug candidates or modifying existing drugs based on the predicted intermolecular interactions of the drug candidates or modified drugs with the structural variants or portions thereof by computationally docking drug molecules with the target protein models; and then, optionally energetically refining the docked complexes; determining the binding interactions between the drug or potential new drug candidate molecules and the models by calculating the free energy of binding of the docked complexes and decomposing the total free energy of binding based on interacting residues in the protein active site or sites deemed important for protein activity.

A variety of methods that include these steps are provided. Such methods have particular application, for example, in predicting patient responses. As noted, patients exhibit variable responses to drugs. For some patients a drug may be very beneficial and achieve a desired response; whereas for other patients, with the same disorder, the same drug will have little or no effect. It is known that individuals as well as groups of individuals exhibit a variety of genetic polymorphisms. As described herein, the presence or absence of such polymorphisms can be correlated with the variability of patient responses to drugs.

It is shown herein that by understanding how genetic poly-morphisms affect 3-D protein structure of a drug target, for example, it is possible to ascertain the interaction of a particular drug with the target in a particular patient or groups of patients. Based upon this interaction, the outcome can be predicted. It will be possible to determine whether a patient will benefit from a drug or be at risk for a particular side effect. It is possible to predict these responses before exposure to the drug. These methods also permit rational design of drugs that can treat various populations or ultimately even individuals. These differences and effects can also be taken into account to design drugs that are not dependent upon a particular polymorphism.

Hence, the knowledge derived from understanding the effects of genetic polymorphisms can be used to develop and apply therapeutics more effectively, make clinical trials more successful, for example, by permitting selection of test subjects with the same polymorphism or with polymorphisms for which the drug is designed to interact effectively.

It is shown herein that it is advantageous to use 3-D molecular structures in drug design rather than to consider primary sequence alone. For example, most drugs target proteins either in the afflicted organism or in a pathogen. Disease, drug action and toxicity are all manifested at the protein level. Although the nucleotide sequences of genetic polymorphisms might appear to be quite different, the resulting protein targets may have similar shapes and, therefore, the protein biological function might be the same. Conversely, although genetic polymorphism sequences might appear similar, the resulting proteins may have critical differences in their 3-D structures that greatly affect biological activity. Thus, use of 3-D protein structure models in such methods provide advantages not heretofor realized. Methods for generating 3-D structures are known to those of skill in the art and are also provided herein.

Once the protein target structural models have been selected, structure-based drug discovery methodologies, for example, computational screening or docking programs and methods (e.g., DOCK (available from University of Ca, San Francisco; and AUTODOCK available from Scripps Research Institute, La Jolla), are used to design biologically-active compounds based on the 3-D structures of the biomolecular receptors. Using these methods, drug designers can identify and computationally rank the various potential clinical drug candidates for maximum efficacy, thereby performing drug discovery in silico and avoiding the tedious time and expense associated with in vitro drug discovery methods.

In addition to drug design applications, the information derived from studying the structures of biological targets can be used to understand and predict biological responses in patients, such as efficacy, toxicity, drug resistance and other pharmacological effects. Since human clinical trials may cost upwards of $100-300 million, it is desirable to predict the outcome to the greatest extent possible for each prospective drug candidate so that the best prospective drug candidates are advanced to clinical trials. As described below, methods are provided herein for selecting populations for clinical trials.

1. Methods for Obtaining Amino Acid Sequences of a Target Protein

Any protein or gene or encoded mRNA that exhibits polymorphisms, herein referred to as the target protein, in structure is contemplated for use herein and for generating the databases as provided herein. The target protein is a protein, polypeptide, or oligopeptide that includes, but is not limited to, receptors, enzymes, hormones, prions, or any such compound with which drugs or other ligands, such as small molecules, peptide agonists, peptide antagonists, other proteins, nucleic acids and other biomacromolecules, interact to bring about a biological response. These target proteins occur in any organism, including plants and animals, eukaryotes and prokaryotes, including pathogens, such as protozoans, parasites, viruses, including DNA and retroviruses, and bacteria. The protein or gene can be one expressed in the organism, such as molecule targeted for drug interaction, or one expressed in a pathogen.

The target gene is one that exhibits polymorphisms (i.e., sequence variations among a population) and the target protein is the product of a gene exhibiting genetic polymorphisms, or sequence variations, as described herein. Any gene or protein that exhibits polymorphisms is contemplated herein. In particular, genes that encode proteins, polypeptides, or oligopeptides that are targets for drug interaction are contemplated herein. The genetic polymorphisms can occur in the genes of pathogens (e.g. viruses, bacteriae, and fungi), parasites, plants, animals, and humans. As such, the sequence a target protein can be obtained by the isolation and analysis of the gene or gene product in samples taken from pathogens, parasites, plants, animals, and humans, most preferably from humans.

The genes or proteins may be isolated from any source, such as animal or plant specimens, or the sequences obtained from any source, including known databases. If starting with gene sequences that include single or multiple nucleotide polymorphisms, the amino acid sequences of the translated proteins can be determined. Protein isolation and sequencing methods are well known to those of skill in the art. Alternatively, samples of the target protein can be obtained and sequenced directly from specimens. Multiple sequence analyses can be performed to determine the exact amino acid variations or mutations resulting from the genetic polymorphisms.

Amino acid sequences of target proteins can also be obtained from data banks and databases (e.g. GenBank, Swiss Prot, PIR) and from publications and other sources in which numerous polymorphisms have been identified and mapped. Samples may be obtained from, for example blood and tissue banks, nucleic acid isolated, genes selected or identified and polymorphims can be mapped from such samples.

2. Generation of 3-D Protein Structural Variant Models

After the amino acid sequences of target proteins are obtained via the means described in section 1, the 3-D structural models of the sequences of native proteins or of the protein structural variants are then determined. They can be determined through experimental methods, such as x-ray crystallography and NMR, and from structure databases, such as the Protein Databank (PDB). Moreover, 3-D structural models can be determined by using any of a number of well known techniques for predicting protein structures from primary sequences (e.g. SYBYL (Tripos Associated, St. Louis, Mo.), de novo protein structure design programs (e.g. MODELER (MSI, Inc., San Diego, Calif.) and MOE (Chemical Computing Group, Montreal Canada) and ab initio methods, see, e.g., U.S. Pat. Nos. 5,331,573, 5,579,250 and 5,612,895), homology modeling, and ab initio computational analysis. Homology modeling, structure determination based upon x-ray crystallographic structures, and ab initio techniques and combinations of these methods are among those preferred herein.

a. Homology Modeling

Homology modeling is based on the relationship between protein evolutionary origin, function and folding patterns. Proteins of related origin and function have conserved sequences and structural features among the members of a homologous family. Using these relationships, a three-dimensional structural model for a protein of unknown structure can be constructed by using composite parts of related proteins in the same family. Where only the primary amino acid sequence of a target protein is known, the sequence can be compared to the sequences of related proteins with known structures (reference proteins), and a model can be built by incorporating the structural attributes of the reference protein together with the sequence of the target protein.

Sequence homology calculations generally require: the amino acid sequence of the target protein; a high resolution structure for at least one, but preferably more, related reference proteins; and any other related amino acid sequences. The reference proteins include structures which are similar to the target protein, either by sequence, fold, function, or which are polymorphisms of the target protein. The more related protein structures and sequences that are available or determined, the more reliable the technique will be at providing an accurate model.

In constructing a protein model using homology modeling, sequence alignment is performed between the target sequence and any known structures within the protein family. Sequence alignment requires determining the similarity between protein sequences by maximizing the number of matches between the sequences while introducing the minimum number of insertions and deletions. Sequence alignment algorithms are well known in the art, and standard gap penalties (i.e., programs that automatically introduce gaps to maximize alignment and then adjust the percentage of identity by applying penalties for gap number and gap length) and other parameters can be selected by the skilled artisan. Additionally, the 3-D structures of the known reference proteins, preferably, are aligned to give the best overall fit for the proteins in the family. This provides indication of structurally-conserved regions, such as regions of the proteins that do not contain insertions or deletions, among the reference structures.

Once the sequences are aligned and the structurally-conserved regions are identified, the coordinates of the reference proteins can be used to construct a 3-D model of the target structure. Coordinates from the protein backbone of the reference proteins are then used to construct the backbone framework for the target protein structure. Side chains can be constructed,.for example, by using side chain coordinates from the reference proteins, searching from a database to obtain side chain conformations that fit in with the existing structural framework or by generating side chains ab initio to establish energetically favorable side chain conformations.

The non-conserved regions of the unknown protein can be constructed, for example, using database searching. A database of known protein structures (e.g., PDB) can be searched to identify variable regions in other proteins that have a high degree of sequence similarity to the target sequence and that fit onto the existing structural framework of the protein model. Algorithms for performing sequence similarity matching and homology model building are well known in the art and are available commercially (available from Molecular Simulations, Inc., Tripos, Inc. and from numerous academic sources).

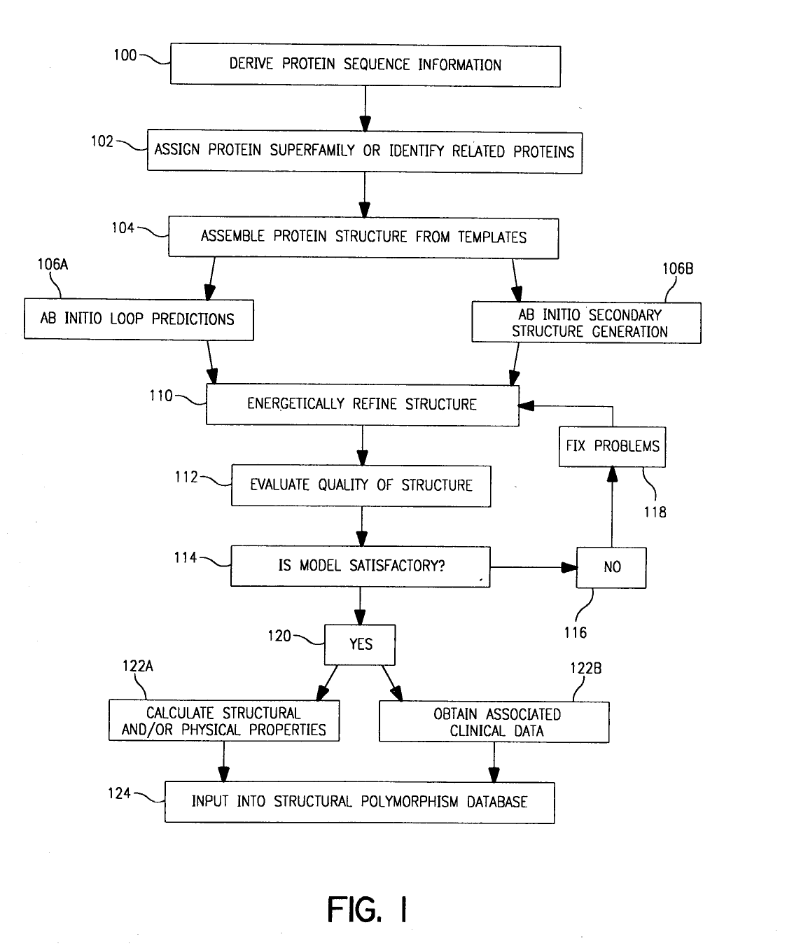

The variable regions can also be modeled by fitting the target sequence to a peptide backbone generated by varying phi and psi angles (e.g., by calculating Ramachandran or Balasubramanian plots, see, Balasubramanian (1974) “New type of representation for Mapping Chain Folding in Protein Molecules,” Nature 266:856-857) or Balaji plots, see, U.S. Pat. Nos. 5,331,573, 5,579,250 and 5,612,895) of the amino acids to give a loop structure that can be integrated into the model structure based on a sterically and energetically reasonable fit (FIG. 1).

In a Balasubramanian plot, the peptide is depicted as a series of different vertical lines, each having solid dots and open circles aligned with the corresponding φ, ψ angle values on the vertical axis, and where each line corresponds to the particular number of the residue having the plotted φ, ψ angles as indicated on a horizontal axis. In the Balaji plot, the values of the φ, ψ angles are shown as the base and tip of a vertical wedge (assuming a vertical angular axis), respectively, with a separate wedge being horizontally positioned on the plot as a function of the residue number of the φ, ψ angles plotted. The Balaji plot replaces the solid dots and open circles of the Balasubramanian Plot with the base of a wedge and the tip of a wedge, respectively; and further replaces the vertical line joining the dots and open circles of the Balasubramanian plot with the body of the wedge.

b. Ab Initio Generation of 3-D Structures

Alternatively, ab initio methods can be used in combination with an existing partial homologous structure to generate unresolved portions of the target structure. Such methods are described, for example, in U.S. Pat. Nos. 5,331,573, 5,579,250 and 5,612,895, which as all patents, applications and publications referenced herein, are each incorporated in their entirety. These methods involve: simulating a real-size primary structure of a polypeptide in a solvent box, i.e., an aqueous environment; shrinking the size of the peptide isobarically and isothermally; and expanding the peptide to its real size in selected time periods, while measuring the energy state and coordinates, i.e., the bonds, angles and torsions of the expanding molecule. As the peptide expands to its full size, it assumes a stable tertiary structure. In most cases, due to the manner in which the expansion occurs, this tertiary structure will be either the most probable structure (i.e., it will represent a global minimum for the structure) or one of the most probable structures. The energy equations used to perform the ab initio simulation are based on the potential energy of the simulated molecule as described using molecular mechanics.

Once a model is built, it can be refined using energy minimization, molecular dynamics calculations, or simulated annealing as described herein. The steric and energetic quality of the structural models is then evaluated by analyzing the structural attributes of the model, such as phi and psi angles (e.g., by calculating Ramachandran or Balasubramanian or Balaji plots), or the energetics of the model, such as by calculating energy per residue or strain energy. If the overall quality of the model is not satisfactory, further iterative energy refinement can be performed until the model is considered to be acceptable (i.e., eₐᵥ<1.5, see below).

A preferred method for generating and refining the structural variant models is illustrated in FIG. 1. First, at block 100 of FIG. 1, protein sequence information, derived genetic polymorphisms, is obtained from the methods described earlier. At block 102, the protein is assigned to a protein superfamily in order to identify related proteins to be used as templates to construct a 3-D model of the protein. If the superfamily is not known, sequence analysis or structural similarity searches can be performed to identify related proteins for use as templates in homology modeling studies, as described herein, as indicated at block 104.

Once the conserved regions of the model are assembled, ab initio loop prediction (Dudek et al. (1998) J. Comp. Chem. 19:548-573) indicated at 106A or ab initio secondary structure generation techniques of block 106B, techniques in which the alignments are adjusted using information on the secondary structure, functional residues, and disulfide bonds as described herein, can be used to complete the model (e.g. U.S. Pat. Nos. 5,331,573; 5,579,250; and 5,612,895). This model, complete with loops, is then subjected to refinement procedures (block 110) based on molecular mechanics, molecular dynamics, and simulated annealing methods. Energetic refinement of the structure can be accomplished by performing molecular mechanics calculations using, for example, an ECEPP type forcefield (Dudek et al. (1998) J. Comp. Chem. 19:548-573) or through molecular dynamics simulations using, for example, a modified AMBER type forcefield (Ramnarayan et al. (1990) J. Chem. Phys. 92:7057-7076. As known to those of skill in the art a modified AMBER (version 3.3) force field is a fully vectorized version of AMBER (3.0) with coordinate coupling, intra/inter decomposition, and the option to include the polarization energy as part of the total energy (see, e.g., Weiner et al. (1986) J. Comp. Chem. 7:230-252). If necessary, the 3-D structures can be dynamically refined, for example, by using a simulated annealing protocol (e.g., 100 ps equilibration, 500 ps dynamics, up to 1000° K, 1 fs data collection).

The refinement process step 110 is used to offset problems that may arise when homology models are not built carefully or when they are built using fully automated methods. Problems that may arise include chain breaks (e.g. consecutive C^{α} atoms are farther apart than the optimum distance of 3.7 to 3.9 Å); distorted geometry (e.g. bond lengths and bond angles are too far from their optimal values); cis-peptide bonds (e.g., incorrect isomerization of the peptide backbone in non-proline residues when it is not required); disallowed backbone and side-chain conformations (e.g., dihedral angles do not satisfy the Ramachandran plot (see, Balasubramanian (1974) Nature 266:856-857) criteria for a fully favorable protein structure conformation); and misfolded loops (e.g. non-homologous loops are generated in unnatural conformations). The refinement procedure 110 removes distortions of covalent geometry by using energetic methdods, converts disallowed backbone and side-chain conformations into allowed ones using simulated annealing methods, conserves protein core structure and secondary structural elements built by homology, and rebuilds unnatural loop constructions (Dudek et al. (1998) J. Comp. Chem. 19:548-573).

For quality control (block 112), the protein structural characteristics, for example, stereochemistry (e.g., phi/psi and side chain angles), energetics (e.g., strain energy), packing profile (e.g., packing factor per residue) and hydrophobic packing are evaluated and required to meet acceptable criteria before the structures are used in further studies or inputted into a structural polymorphism database. Quality control using strain energies entails computing normalized residue energies (NREs) based on the equation:

e_{I}=[E(i,X)−E_{AV}(X)]/E_{SD}(X), where

E(i,X) is the energy of interactions of amino acid X in position i with protein environment and solvent;

E_{AV}(X), E_{SD}(X) is the average residue energies and their standard deviations calculated for 20 amino acids in more than 100 high-quality crystal structures; and

NREs characterize how favorable the interactions of each residue are within the protein environment (Majorov and Abagyan, (1998) Folding & Design 3:259).

The average NRE characterizes the overall quality of a protein structure and is defined as:

eₐᵥ=(1/N) Σᵢe_{I}, where

eₐᵥ≦0.5 denotes high-resolution X-ray crystal structures;

eₐᵥ≦1.0 denotes good as NMR and theoretical models; and

eₐᵥ≧1.5 denotes structures that require further refinement.

After the quality of structure is determined at block 112, the model is checked at block 114 to determine if it is satisfactory. If the overall quality of the model is not satisfactory, a “No” outcome at block 116, then remedial action is undertaken to fix problems at block 118, including further iterative energy refinement (block 110), and repeated checking (block 114). The refinement and evaluation is repeated until the model is considered to be acceptable, a “Yes” outcome at block 120, whereupon structural and/or physical properties (e.g. energetics and phi/psi angles) are calculated at block 122A and clinical data (if available) is obtained at block 122B. The model is then inputted into a structural polymorphism database at block 124.

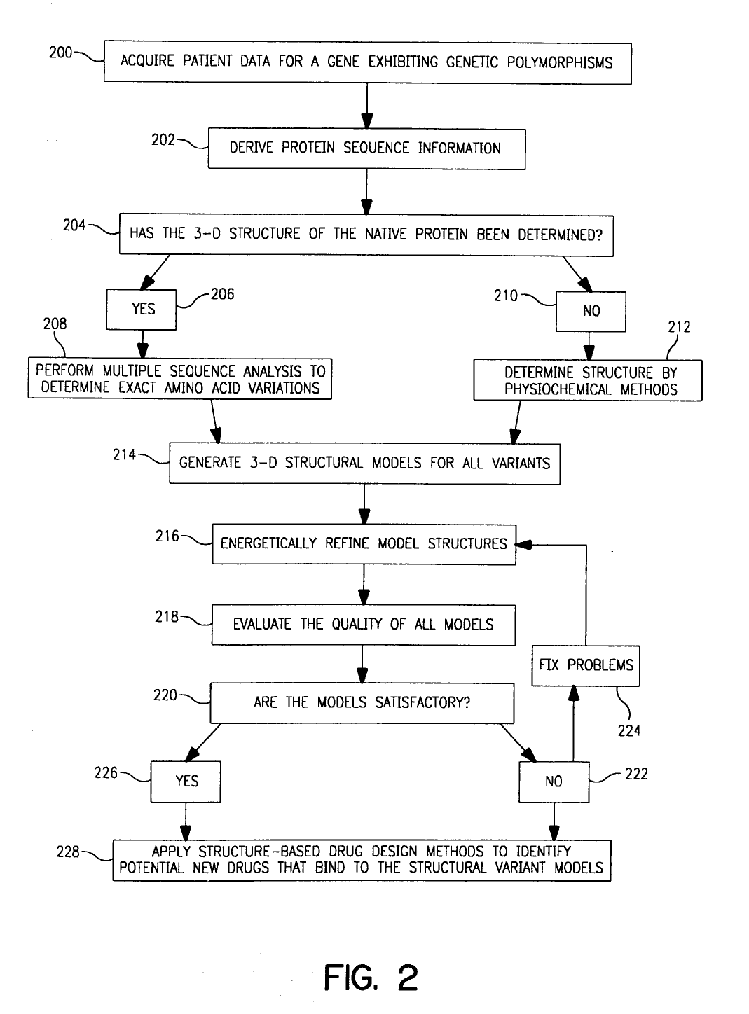

FIG. 2 shows an exemplary method for generating structural variant models derived from genetic polymorphisms and using them in structure-based drug design studies. At the block numbered 200, patient data is acquired for a gene that exhibits genetic polymorphisms. Protein sequence information is then derived, at block 202. A check is made for determination of the 3-D structure of the native protein. If the 3-D structure has been determined, a “Yes” outcome at block 206, then a multiple sequence analysis is performed at block 208 to determine the exact amino acid variations for the structure. If the 3-D structure has not been determined, a “No” outcome at block 210, then the structure is determined using physiochemical methods at block 212.

Next, at block 214, the 3-D structural models for all variants are generated. A refinement process is then completed at block 216 for the structural models. As noted above in connection with FIG. 1, the process involves subjecting each model, complete with loops, to refinement procedures based on molecular mechanics, molecular dynamics, and simulated annealing methods. As before, the energetic refinement of the structure can be accomplished by performing molecular mechanics calculations using an ECEPP type forcefield (Dudek et al. (1998) J. Comp. Chem. 19:548-573), or through molecular dynamics simulations using, for example, a modified AMBER type forcefield (Ramnarayan et al. (1990) J. Chem. Phys. 92:7057-7076), where a modified AMBER (version 3.3) force field is a fully vectorized version of AMBER (3.0) with coordinate coupling, intra/inter decomposition, and the option to include the polarization energy as part of the total energy (Weiner et al. (1986), J. Comp. Chem. 7:230-252). If necessary, the 3-D structures can be dynamically refined, for example, by using a simulated annealing protocol (e.g., 100 ps equilibration, 500 ps dynamics, up to 1000° K, 1 fs data collection).

At block 218, a quality evaluation is performed for all the models. As described in connection with the quality evaluation process in FIG. 1, the evaluation at block 218 involves evaluating the protein structural characteristics, for example, stereochemistry (e.g., phi/psi and side chain angles), energetics (e.g., strain energy), packing profile (e.g., packing factor per residue) and hydrophobic packing, which must meet acceptable criteria before the structures are used in further studies or inputted into a structural polymorphism database.

After the model quality is determined, at block 220 the models are checked to determine if they are satisfactory for further use. If a model is not satisfactory, a “No” outcome at block 222, then the problems are identified and solved with remedial action at block 224. The remedial action may include further iterative energy refinement at block 216 and repeated checks of model quality at block 218. Once the models are satisfactory, a “Yes” outcome at block 226, structure-based drug design methods are applied at block 228 to identify potential new drugs that bind to the structural variant models. The drug design methods are described further below.

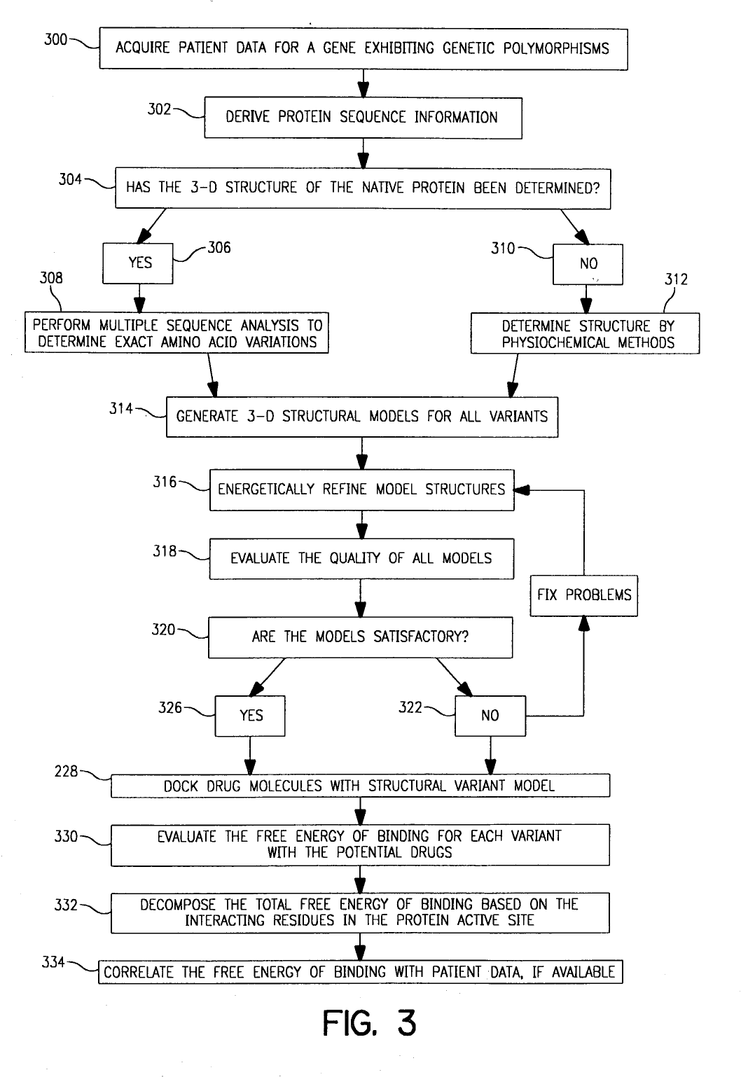

FIG. 3 shows another exemplary and alternative method for generating structural variant models derived from genetic polymorphisms and using them in structure-based drug design studies. The process of FIG. 3 is similar to the process of FIG. 2 from the initial process at block 300 of acquiring patient data for a gene that exhibits genetic polymorphisms through the process of obtaining models that are satisfactory (a “Yes” outcome at block 326). Thus, block numbers in FIG. 3 from 300 through 326 that correspond to FIG. 2 blocks numbered from 200 thorough 226 refer to similar operations. Unlike FIG. 2, however, the process illustrated in FIG. 3 then involves docking operations.

At block 328, once the models are determined to be satisfactory, drug molecules are docked with the structural variant models. Next, at block 330, the free energy of binding is evaluated with the potential drugs under study for each structural variant model. At block 332, the total free energy of binding is decomposed, based on the interacting residue in the protein active site. Lastly, at block 334, the free energy of binding is correlated with patient data, if the data is available. Thus, the 3-D structural data is employed in drug design. Details of using such structural data in drug design are described further below.

C. Crystal Structures

The crystal structure of any protein can be determined empirically and the resulting coordinates used as the basis for determing structures of variants. Such structures are often known (see, e.g., Kohlstaedt et al. (1992) Science 256:1773-1790 for a crystal structure of HIV-1 RT bound to a ligand).

3. Use of 3-D Structural Variant Models in Drug Design

The structural differences in protein structural variants that arise due to genetic polymorphisms can have profound effects on biological activity. Because of the structural differences among the variants, they may have different physical or reactive properties and therefore may exhibit different biological activities. These differences may include, for example, different responses to a given drug, so that a drug which works well in a patient with one particular genetic polymorphism may not work as well in another patient exhibiting a different polymorphism.

The 3-D molecular structures of drug targets derived from genetic polymorphisms can be used in structure-based drug design studies to greatly advance the development of new pharmaceuticals. Relational databases of these 3-D structures that are derived from samplings of genetic polymorphisms over a patient population or a cross-section of the population can be used to design potential drugs in order to optimize effectiveness for the particular population.

The structures and databases described herein can provide information that is useful, for example, in designing a drug that is effective in the greatest percentage of the population. It is desirable that a given drug is effective in the largest percentage of the population, since such a drug is likely to have the greatest clinical utility and thus the greatest commercial value. A drug with superior performance properties is sometimes referred to as a “best in class” drug and is highly prized by pharmaceutical companies since this heralds market leadership and the likelihood of commercial success. The databases and methods described herein can be used to determine 3-D protein structures for drug targets that are associated with particular genetic polymorphisms and to use the structures in drug design studies for design and optimization of candidate drugs that exhibit activity over the broadest patient population.

Genetic polymorphisms may result in target protein structural variants in which drug efficacy correlates with specific populations or subpopulations. In some cases, it might be desirable to target drug design or drug therapy toward a specific patient population, such as a particular race, gender, or age group, affected by a certain disease or condition or toward those having a specific genetic polymorphism. The information derived from comparing the 3-D structural variants arising from different genetic polymorphisms may be useful for understanding why drugs are active or inactive in different subpopulations, or for assisting in developing new drugs to maximize efficacy across specific populations.

a. Selection of Relevant Structural Variants

The structural variant models in the structural polymorphism database provided herein can be used to design new drugs or to select a drug therapy that would be appropriate for a patient exhibiting a particular genetic polymorphism. As it may not be possible for a drug to work equally well for all polymorphisms, and thus all patients, representative structural variants can be selected for use in drug design studies in order to maximize biological activity based on genetic polymorphisms.

In some cases, structural variants are analyzed to determine the common structural features that are conserved through the selected models. These conserved features are used as a basis for drug design. In some cases, the structural variant corresponding to the genetic polymorphism occurring most commonly in a population can be selected for use in identifying drugs that would be effective in the greatest percentage of the population. Optionally, structural variants corresponding to a relevant subpopulation, such as a particular gender, age, race, or other characteristic, can be selected for use in designing drugs that are active in that subpopulation. In other cases, individual structural variant models can be selected for use in designing drugs that are specifically active against one target in one individual arising from a particular genetic polymorphism. Additionally, model structures that represent variants derived from patients that receive a specific treatment regimen or exhibit a particular clinical response (e.g. drug resistance) to a given drug are used as bases for drug design.

The relevant structural variants may be identified using the structural analysis tools described herein, optionally in combination with database and statistical analysis tools that permit a complete analysis and comparison of the molecular structures and properties of the structural variants. The structural variants selected based on the criteria including, but not limited to, those listed above are used in drug design.

b. Drug Design

Once the protein target structural models have been selected, structure-based drug discovery methodologies, for example, computational screening or docking (e.g., DOCK (available from University of Ca, San Francisco; and AUTODOCK available from Scripps Research Institute, La Jolla and others referenced herein or known to those of skill in the art), can then be used to design biologically-active compounds based on the 3-D structures of the biomolecular receptors.

Using these methods, drug designers can identify and computationally rank various potential clinical drug candidates for maximum efficacy, thus cutting the time and expense associated with drug discovery. The preferred design of drug candidates or the modification of existing drugs is based on the intermolecular interactions between the drug candidate or modified drugs and the selected structural variants predicted by computationally docking drug molecules with the target protein models; energetically refining the docked complexes; determining the binding interactions between the drug or potential new drug candidate molecules and the models by calculating the free energy of binding of the docked complexes and decomposing the total free energy of binding based on interacting residues in the protein active site or sites deemed important for protein activity.

C. Computational Docking

Methods for using the structural variant models to design potential new drugs or to aid in the selection of a drug therapy based on the interactions of selected small molecules with the particular variants are provided. Structure-based drug design experiments, such as computational screening or docking studies, calculation of binding energies or analysis of steric, electrostatic or hydrophobic properties of the resulting structural variant models, can be performed on selected structural variant models to aid in the understanding of observed biological activities or to determine new potential drug candidates to bind to the particular target.

In a typical computational docking protocol, the active site, or sites deemed important for protein activity, of the protein model is defined. A molecular database, such as the Available Chemicals Directory (ACD) or any database of molecules, is screened for molecules that complement the protein model. Solvation parameters are factored in (see, e.g., Shoichet et al. (1999) PROTEINS: Structure, Function, and Genetics 34:4-16). In these computational docking studies, drugs or drug candidates are fitted to the structural variant models based on complementary interactions (e.g., steric, hydrophobic, or electrostatic interactions). Methods for performing such studies are well known and software tools for performing the calculations are widely available (M. Lambert, “Docking Conformationally Flexible Molecules into Protein Binding Sites” in Practical Application of Computer-Aided Drug Design, Charifson, Ed., Marcel Dekker, NY, pp. 243-303; Kuntz (1992) Science 257:1078-1082; Kuntz et al. (1982) J. Mol. Biol. 161:269-288; Stewart et al. (1992) Med. Chem. Res. 1:439-443; Shoichet et al. (1993) Science 259:1445-1450; Shoichet et al. (1991) J. Mol. Biol. 221:327-346).

New potential drug candidates can be designed by identifying potential small molecule drugs that can bind to a particular structural variant. This is accomplished, for example, by methods including, but are not limited to, methods for electronic screening of small molecule databases as described herein, methods involving modifying the functional groups of existing drugs in silico, methods of de novo ligand design. Methods for computationally desiging drugs are known to those of skill in the art and include, but are not limited to, DOCK (Kuntz et al. (1982) “A Geometric Approach to Macromolecule-Ligand Interactions”, J. Mol. Biol., 161:269-288; available from University of Ca, San Francisco); and AUTODOCK (see, Goodsell et al. (1990) “Automated Docking of Substrates to Proteins by Simulated Annealing”, Proteins: Structure, Function, and Genetics, 8, pp. 195-202; available from Scripps Research Institute, La Jolla); GRID (Oxford University, Oxford, UK); CAVEAT (UC Berkeley, Calif.), LEGEND (Molecular Simulations, Inc., San Diego, Calif.); LUDI (Molecular Simulations, Inc., San Diego, Calif.); HOOK (Molecular Simulations, Inc., San Diego, Calif.); CLIX (CSIRO, Australia); GROW (Upjohn Laboratories, Kalamazoo); others including HINT, LUDI, NEWLEAD, HOOK, PRO-LIGAND and CONCERTS (see, M. Murcko, “An Introduction to De Novo Ligand Design” in Practical Application of Computer-Aided Drug Design, Charifson, Ed., Marcel Dekker, NY, pp 305-354), methods based on QSAR (quantitative structure-activity relationships, QSAR and Drug Design: New Developments and Applications, Fugita, Ed., (1995) Elsevier, pp 3-81; 3D QSAR in Drug Design, Kubinyi, Ed., (1993) Escom, Leiden), and other methods known to those of skill in the art for determining molecules that have optimal binding interactions with a selected target.

The docked complexes, if needed, are further refined energetically to optimize geometries within the binding site and to select the best structure from a set of possible structures, using molecular mechanics, molecular dynamics, and simulated annealing techniques, including those described herein and others that are known to those skilled in the art.

d. Free Energy of Binding Studies

After the computational docking step, the free energy of binding of the docked complex is calculated, and the total free enegy of binding is decomposed based on the interacting residues in the protein active site or sites deemed improtant for protein activity. Analyses of the binding energies are needed to identify drug candidates. If needed or desired, the free energy of binding of different drugs or potential drugs to each structural variant model can be calculated by substracting the free energy of the non-interacting protein and drug from the free energy of the protein-drug complex. The total free energy of binding is decomposed into its various thermodynamic components, e.g. enthalpic and entropic components, based on the interacting residues in the protein active site in a solvated model to characterize the structural and thermodynamic features in the mode of drug binding and to determine the contribution of the solvent (see, e.g., Wang et al. (1996) J. Am. Chem. Soc. 118:995-1001; Wang et al. (1995) J. Mol. Biol. 253:473-492; Ortiz et al. (1995) J. Med. Chem. 38:2681-2691, which describes a computational method for deducing QSARs from ligand-macromolecule complexes). Following the computational drug design protocol described herein, any potential new drugs that are identified can be synthesized in, for example, industry or academia, and subjected to further biological testing, such as in vitro studies or pre-clinical and clinical in vivo testing.

Based on the predicted intermolecular interactions of the drugs or modified drugs with the structural variant models from binding studies, potential drug candidates that are specific for a protein with a selected polymorphism or that specifically interact with all proteins exhibiting the polymorphism can be identified.

It is also possible to individualize drug design or drug therapy by determining the structural variants associated with a particular patient and then designing or screening drugs or potential drugs to maximize efficacy in that subject or in a subpopulation that exhibits the same genetic polymorphism. The variants may also be used to track polymorphic variations in infectious organisms, such as viruses. For example, the human immunodeficiency viruses (HIVs) reverse transcriptase and protease have served as drug targets (see, Erickson et al. (1996) Ann. Rev. Pharmacol. Toxicol 36:545-571); their three-dimensional structures are known (see, e.g., Nanni et al. (1993) Perspectives in Drug Discovery and Design 1:129-150; Kroeger et al. (1997) Protein Eng. 10:1379-1383). The clinical emergence of drug-resistant variants of these viruses has limited the long-term effectiveness of drugs targeted against these enzymes.

As noted, these enzymatic proteins in order to preserve function must exhibit conserved 3-D structures. The methods herein permit design of drugs specific for the conserved regions of the 3-D structures. They also permit selection of drug regimens based upon the alleles expressed. Hence, methods for designing HIV enzyme-specific drugs are provided. Flow charts illustrating exemplary alternative embodiments using protein 3-D structures derived from genetic polymorphisms in structure-based drug design studies are provided (see, FIGS. 2 and 3). In the flow charts depicted in these figures, the drug design includes structure-based drug design methods (see, FIG. 2) and computational docking of drugs with structural variants, evaluation of the binding energy of the docked complexes, and correlation of the binding energy with patient data such as age, gender, race, drug treatment history, and any other pertinent information that is available (see, FIG. 3). The data generated by this computer-based method can be stored in a database, such as, for example, in a relational database. The resulting database can be screened using searching tools to select potential drugs and therapeutic agents that bind to or exhibit biological responses towards target proteins.

C. Applications of Computer-Based Methods

As discussed above, the computer-based methods provided herein include some or all of the steps of obtaining one or more, preferably two or more, amino acid sequences of a target protein that is the product of a gene exhibiting genetic polymorphisms; generating 3-dimensional (3-D) protein structural variant models from the sequences; and based upon the structures of the 3-D models, designing drug candidates or modifying existing drugs based on the predicted intermolecular interactions of the drug candidates or modified drugs with the structural variants by computationally docking drug molecules with the target protein models; energetically refining the docked complexes; determining the binding interactions between the drug or potential new drug candidate molecules and the models by calculating the free energy of binding of the docked complexes and decomposing the total free energy of binding based on interacting residues in the protein active site or sites deemed important for protein activity. There are numerous applications of these methods, which include structure-based drug design and drug testing; selection of clinically relevant populations for drug testing and other such methods.

1. Genetic Polymorphisms and Structure-Based Drug Design

As noted above, structure-based drug design is an increasingly useful methodology that has made a great impact in the design of biologically active lead compounds. Drug designers can design and screen potential new drugs via computational methods, such as docking or binding studies, before actually beginning patient testing. The drugs designed by such methods, and also those identified by traditional methods of drug discovery, are then tested in clinical trials. Among those that show efficacy for a particular indication and low toxicity ultimately are approved for use. It is found, however, that not all patients with a particular indication respond uniformly to the drugs. The drug may not be efficacious or side-effects may be pronounced.

The methods provided herein, represent a further advance in the use of rational drug design methods. As described herein, polymorphic variation has an effect upon the 3-D structure of encoded proteins. As a result, drugs interact with variants differently, leading to differential responses in the population as a whole. A new approach to drug design and testing is provided herein. This methods involves identifying polymorphisms and determining 3-D resulting structures, which are then used in methods, including, computational drug design, in the selection of patient populations, in designing treatment protocols and in other applications.

2. Drug Resistance

Methods for understanding and overcoming drug resistances by using 3-D protein model structures resulting from multiple genetic polymorphisms or mutations in an infectious agents, such as viruses, bacterial and other pathogenic agents are provided. Also provided are methods for using this information in drug design studies.

In the case of infectious organisms or other replicating or mutating agents, such as flu, HIV, rhinovirus or biological warfare agents, some polymorphisms or mutations may arise over time which convey resistance or susceptibility to specific drug therapy, for example, by altering the drug target structure or physical properties so that a specific drug or therapy, such as an antibiotic or vaccine, may no longer be able to bind to or otherwise interact with the target protein to exert its desired biological effect. For certain infectious agents, such as HIV, genetic polymorphisms in certain genes give rise to drug resistance as the virus mutates (see, e.g., Erickson et al. (1996) Annu Rev. Pharmacol. Toxicol. 36:545-571).

Where drug resistance that arises from mutations or poly-morphisms is observed, the methods described herein can be used to develop new drugs that overcome the resistance. For example, once drug resistance is observed, the structure associated with the resistant polymorphism can be determined and used in further drug design studies to suggest new drugs or modifications to the existing drug that will restore biological activity by targeting different mutants or that will target multiple mutants simultaneously.

The model structures can also be used to correlate drug resistance in infectious diseases with the structural variants derived from genetic polymorphisms. Here, the 3-D structure of the virus or other drug target is determined for the particular variant model against which the drug was effective. When drug resistance arises due to a genetic polymorphism, a model for the structure variant associated with the resistant organism can be generated, and a new drug can be designed or modifications can be made to the existing drug to overcome the resistance.

For example, samples of the mutating organism can be obtained over time and structural models for the resulting proteins can be generated. These models can then be used to design new drug therapies that are active against the mutated organism. Multiple drug resistant structures can be analyzed to obtain an average structure or to identify common structural features in order to design new drugs that have the broadest spectrum of activity against multiple mutations.

Such structural information is useful in designing effective drug therapies to overcome resistance or to develop drugs that are effective over a range of genetic polymorphisms and thus work for the maximum number of patients.

3. Identification of Conserved Structural Features or Pharmacophores

If common structural features are observed over a range of protein targets that are derived from genetic polymorphisms, these common features may be used to design a drug that is effective with a variety of genetic polymorphisms and thus many patients. The retention of certain common structural features over a large number of genetic polymorphisms suggests that those features may not be mutatable because the conserved structure may be essential to protein function, e.g., to the viability of an infectious organism or virus. Such conserved structural elements are prime targets for structure-based drug design, e.g., anti-infective or antibiotic drug design, and can lead to highly effective therapies.

The common structural features can serve as a basis for structure-based drug design, for example, by serving as a scaffold for building a receptor model into which potential drug candidates can be docked or as a pharmacophore query for screening a library of physical or virtual chemical or biochemical molecules to identify compounds that match the pharmacophore template and, thus, are potential drug candidates.

Analysis of 3-D protein structural variants derived from genetic polymorphisms to identify the common structural features over a large number of structural variants can aid in the design of drugs that are active over a broad range of genetic polymorphisms, such as in a large number of patients or against drug resistant targets.

In comparing sets of related protein structures, such as those with the same biological function or those resulting from genetic polymorphisms, certain parts of the structural framework are often found to be conserved, while other parts vary among the proteins. Mutations that occur in the conserved regions of the structure can have significant effects biological activity. For example, in viruses, the conserved features can be essential to protein function and, thus, to the viability of the infectious organism or virus. Identifying the conserved structural features over a range of structures often gives insight into which structural features are necessary for biological activity and are therefore non-mutatable. By analyzing a number of structural variants derived from genetic polymorphisms that exhibit drug resistance, it is possible to identify or design drugs that interact best with the common structural features in all of the variants. Using these features in structure-based drug design studies leads to the identification of drugs that retain biological activity despite multiple mutations, or polymorphisms, and could help to overcome the problem of drug resistance.

In certain preferred embodiments, new potential drug candidates can be identified using the structural variant models by identifying pharmacophores or conserved features in the protein structural variant models and using this structural information to identify small molecules that would bind to the structural variant models.

Using structural comparison tools described herein, the common structural features that are conserved across a range of structural variant models of a given protein based on different genetic polymorphisms can be identified. To do this, multiple structural variant models are compared, generally by superimposing the coordinates of one variant model onto those of one or more other variants and observing the structural fit. Such functionality is commonly found in molecular graphics or homology modeling packages. Once the optimum fit of structures is performed, then the structural features that are present throughout the structural variant models can be identified and used as the basis for drug interactions in structure-based drug design studies. For example, the pharmacophores or conserved features can be specified as database queries and a library or database of small molecule structures can be searched to identify new lead compounds to bind to the pharmacophores. Alternatively, other structure-based ligand design strategies can be employed to design lead compounds or to identify modifications to be made to existing drugs to improve biological activity.

4. Identification of Compensatory Structural Changes

Certain proteins, for example, viral proteins or other infectious organisms, may harbor multiple genetic polymorphisms. Since each genetic polymorphism can give rise to slight changes in structure, some, and over time, many, additional genetic polymorphisms may cause changes in the protein structures that significantly affect biological activity. These structural changes could result in, for example, different dynamical behavior, alteration in enzyme kinetics or differences in substrate recognition, which can significantly alter drug response. For example, a mutation for one drug compound can suppress a mutation to a second drug due to compensatory effects. In these cases, a drug which is predicted to be ineffective for a given patient based upon the single nucleotide correlation may, in fact, be effective as a result of these changes.

Because mutations are so frequent in AIDS and other viruses, few sequences are exactly the same in different patients. Thus, it is difficult or inconclusive to generate multiple mutation sequence correlations for drug resistance. If each patient has a different viral sequence due to a high viral mutation rate, then no sequence correlation is even possible in such cases.

The methods described herein can be used to study the effects of multiple genetic polymorphisms on a resultant protein structure. Multiple mutations are common in AIDS and other viruses, which makes sequence correlation difficult. By observing the structural effects of the mutations on the resulting protein, it is possible to look at the net effect of all structural changes and to consider the overall structure of the protein in drug design studies. For example, a mutation might occur in the active site, or site of drug action, in a protein. Additionally, there may be related mutations in other parts of the protein structure, which might not be identified from a single point mutation correlation. These related mutations could have an effect on biological activity of the protein. By looking only at the active site, it might be predicted that a drug or potential drug would not bind to the protein. The additional mutation, however, might cause compensatory structural changes in the protein structure that alter its properties in a way that restores biological activity.

By computing 3-D protein structures from gene sequences containing multiple polymorphisms, it is possible to more accurately predict the effect of multiple sequence mutations on protein structure and, thus, to obtain a better correlation between sequence and drug resistance than by considering sequence correlations alone. This information can be useful, for example, in understanding drug resistance and can aid researchers and clinicians in developing new drug therapies to overcome drug resistance.

The structures that are derived based on multiple generic polymorphisms can be used in structure-based drug design studies to provide frameworks, or scaffolds, into which drug or potential drug molecules can be docked. This permits the design of drugs that are active against a wider range of structural variants, thus, in more patients or against a range of drug resistant proteins.

5. Clinical Applications

A knowledge of the repertoire of structural differences arising from genetic polymorphisms across the human population or specific subpopulations can provide insight into the differing biological responses in patients based on their genetic differences. For example, where clinical data are available for patients having particular genetic polymorphisms, this information can be associated with the 3-D protein structural variants and used to find correlations between polymorphisms and observed drug responses.

The methods provided herein can be used to design drug therapies that bring about favorable clinical responses (or eliminate unfavorable effects) in patients, to identify pharmacological effects of drugs in different patient subpopulations (e.g. age, race, gender) and to simulate clinical trails to increase the probability that the trials will yield optimal results.

Because of the high cost of clinical trials, such studies are generally focused on small patient populations. The structural analysis tools described herein permit the extension of clinical trials to cover patient populations not specifically included in the study. This is accomplished through correlation of the structural variants derived from genetic polymorphisms with clinical responses.

The molecular structures and databases described herein can also find application in the understanding and prediction of clinical or pharmacological drug responses, for example, efficacy, toxicity, dose dependencies or side effects in patients. For example, relational databases containing 3-D protein structural variants can provide a means for managing and using the information to understand and predict clinical responses in patients.

In other embodiments, observed clinical data from patients in a clinical trial can be associated with the structural variant models for each genetic polymorphism exhibited in the clinical subjects, for example, in a structural polymorphism relational database. The correlation between the structural variants and observed clinical effects can then be utilized to predict clinical outcomes in patients that did not participate in the clinical trial. For example, a structural variant model can be generated for a patient based on a genetic polymorphism exhibited in the patient, and the database can be mined to identify structurally similar variants for which clinical results are known. Structural similarity can be determined, for example, by superimposing the structures and measuring the RMS (root mean squared) differences between the structures or by using pattern matching or motif searching algorithms. The results can be used to predict clinical responses in the patient based on the clinical data associated with the structurally similar variants.

The predicted correlations can also be used to aid in the design of subsequent clinical trials. The follow-up trials can be made more effective through the judicious selection of patients with given genotypes (i.e., those exhibiting the same genetic polymorphisms), as guided by the structurally predicted outcomes. For example, a clinical trial can be designed based on a subpopulation of clinical subjects which exhibit a specific genetic polymorphism (i.e. structural variant) to demonstrate the effectiveness of a given therapeutic on a targeted population.

In other embodiments, the methods provided herein can be used in the selection of drug therapies for patients exhibiting a particular genetic polymorphism. This is accomplished by generating the structural variant model associated with the polymorphism, docking drug molecules that might be used to treat the patient into the structural variant model and calculating the binding energies of each drug with the variant. The results of docking or free energy calculations can be correlated to clinical data, for example, patient population (e.g., ethnic background, race, sex, age), treatment regimen, patient response to a particular drug or duration of treatment. The binding energies can be compared, for example, to determine which drug would best bind to the variant in order to identify the drug that could best be used to treat the patient to optimize biological activity.

### D. CREATION OF 3-D STRUCTURAL POLYMORPHISM DATABASES

The above-noted methods all rely upon the use of databases of nucleic acid sequences. Any such database known to those of skill in the art may be employed; numerous such databases are publically available (e.g. the Stanford HIV database). The Stanford HIV database is hierarchal database with information about HIV patients who received or did not receive protease inhibitor treatments, patient-dates, isolates, sequences, hyperlinks to MEDLINE and GenBank abstracts, and art. This database, however, does not contain 3-D protein structures of any proteins including HIV reverse transcriptase (RT) and HIV protease (PR; see, e.g., Shafer et al. (1999) Nucleic Acids Res. 27:348-352, Shafer et al. (1999) J. Virol 73:6197-6202, http://hivdb.stanford.edu/hiv, Richter (Jan. 20, 1999) “AIDS drugs found to be effective in the world's most common HIV strains).

Databases of sequences and associated information may also be generated as described herein by obtaining samples and sequences from a variety of sources. In all instances, further databases are generated by then calculating 3-D structural models of the encoded proteins or relevant portions, such as active binding sites, thereof, from the nucleic acid sequence information. It is these databases of nucleic acid sequence and/or primary protein sequence and the associated 3-D structure that are provided herein and that are used in the all of the methods, except for the computational phenotyping discussed below, which does not require a database, provided herein. Hence databases containing computationally determined 3-D structures of polymorphic proteins or portions thereof are provided herein. These databases serve as tools in a variety of methods, including those provided herein.

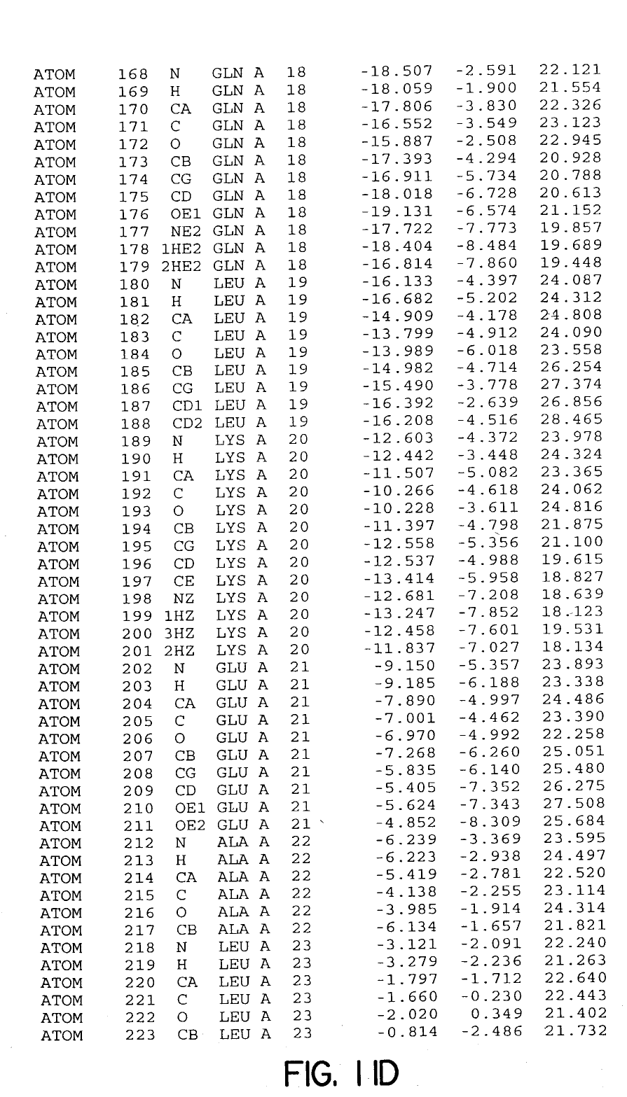

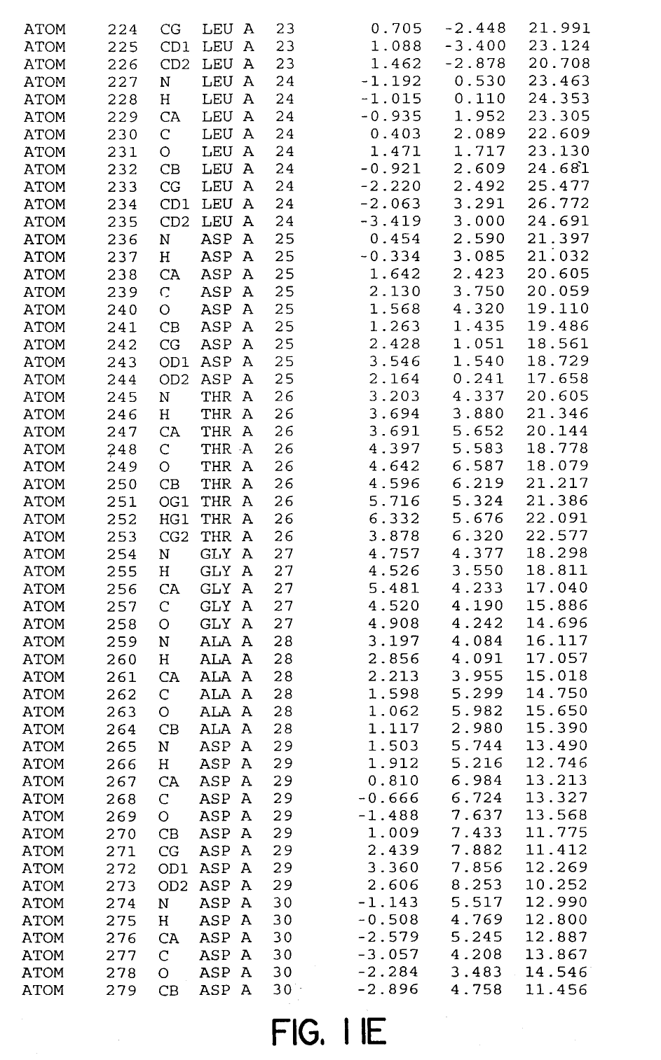

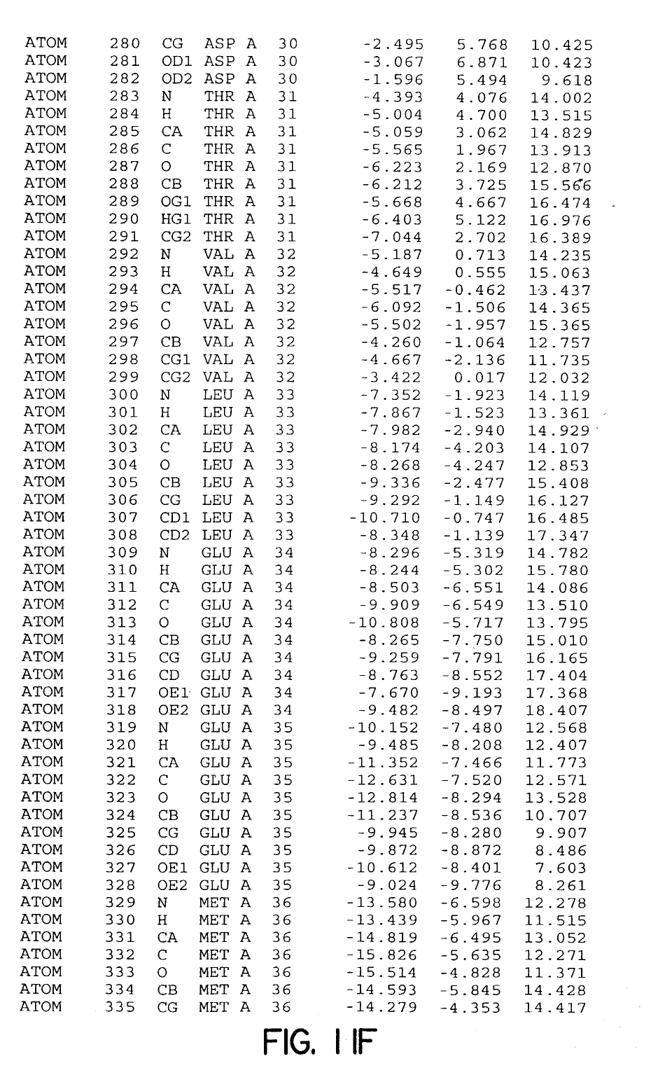

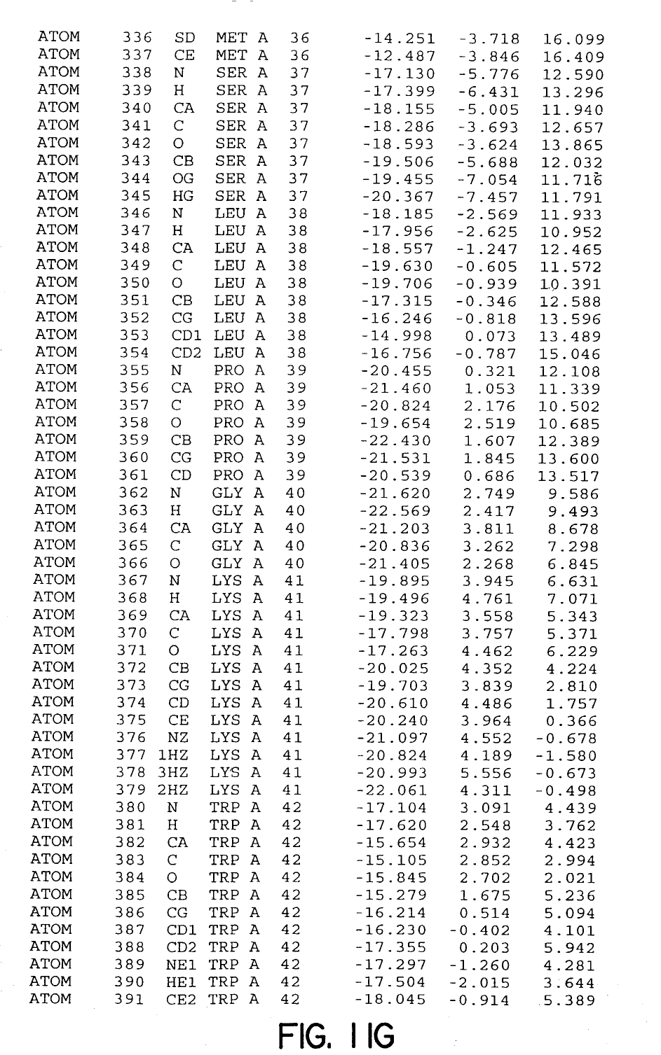

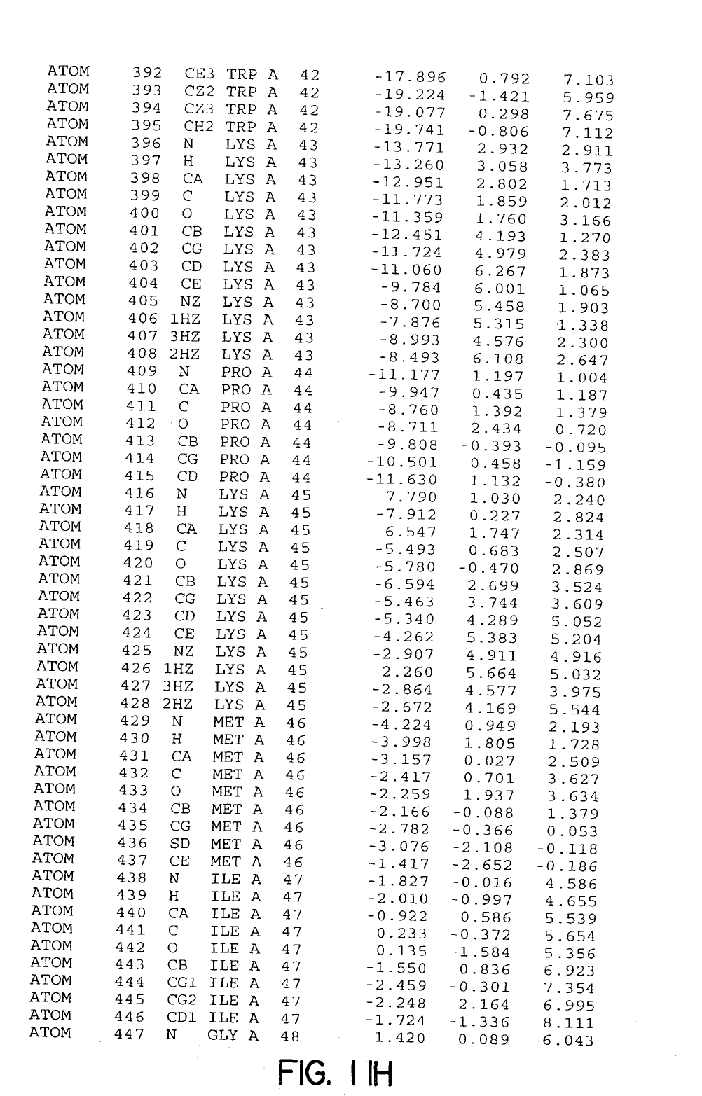

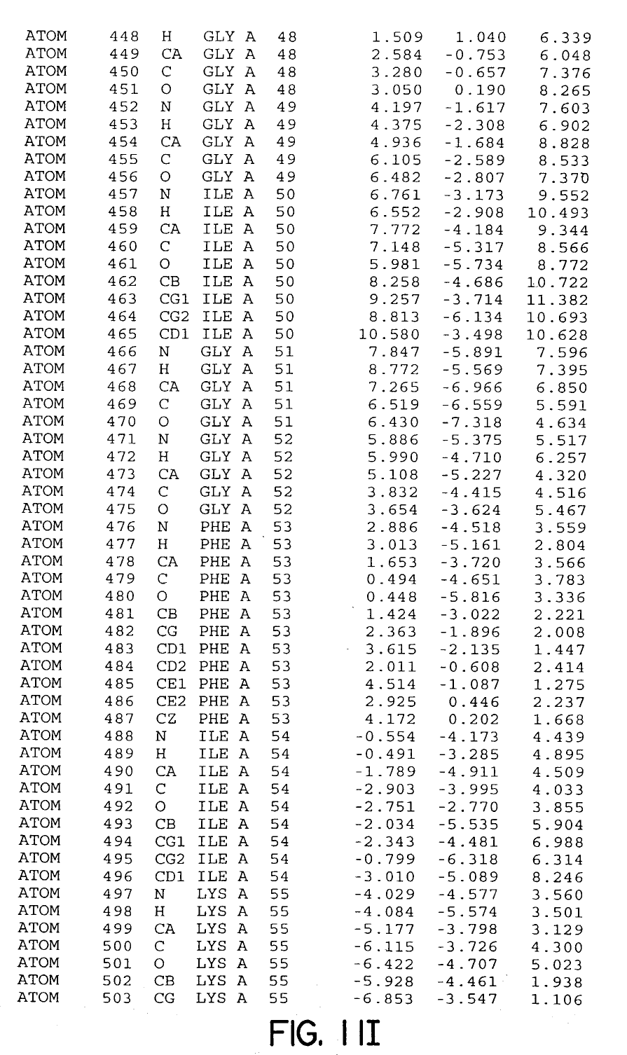

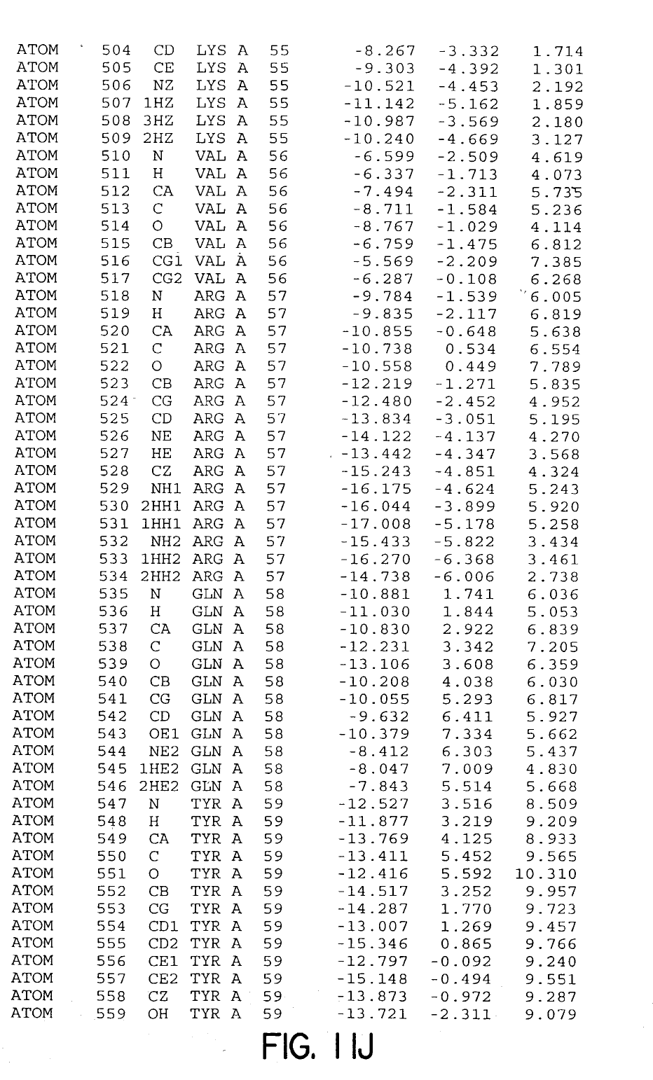

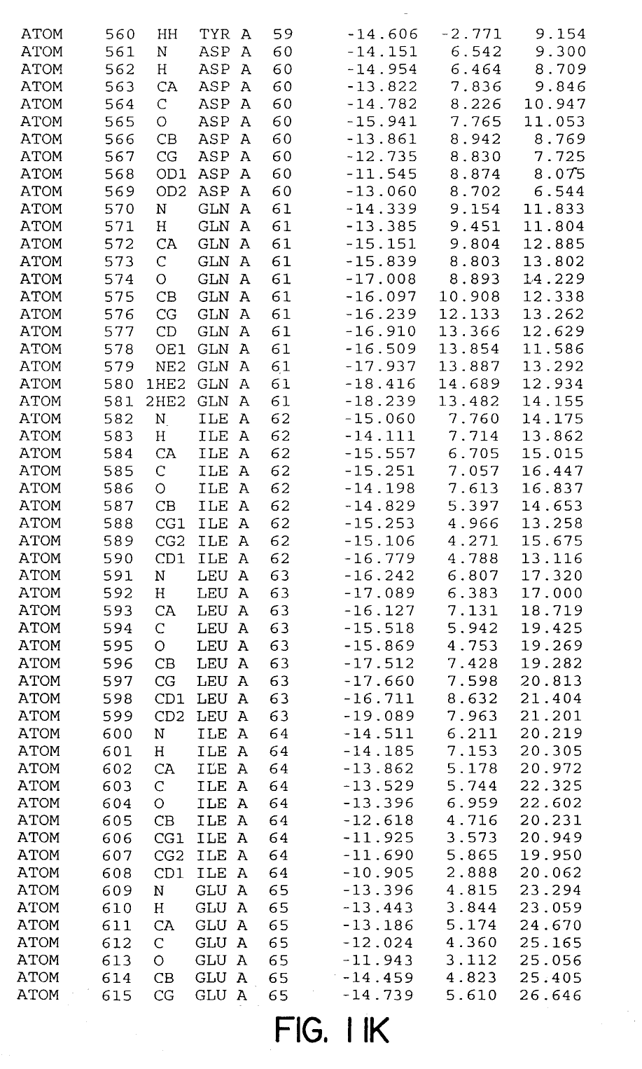

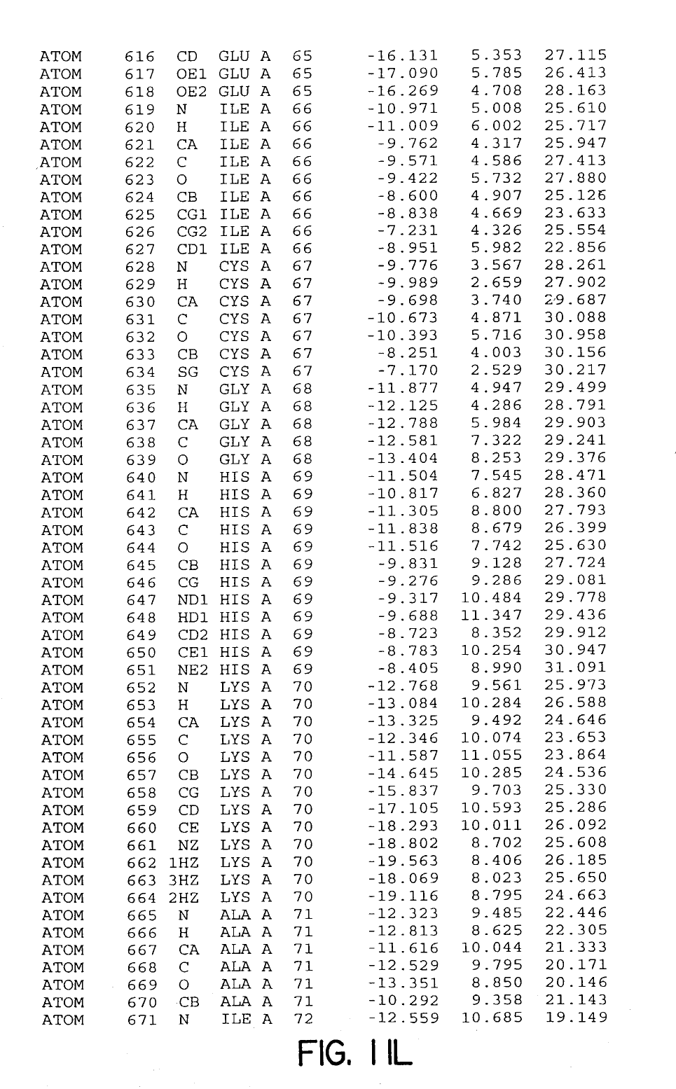

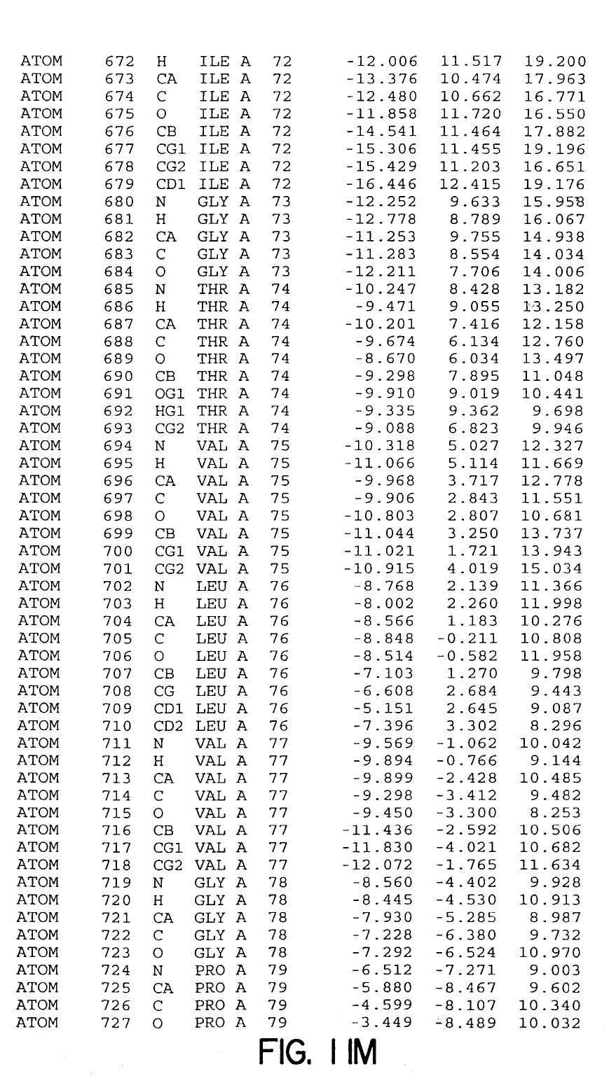

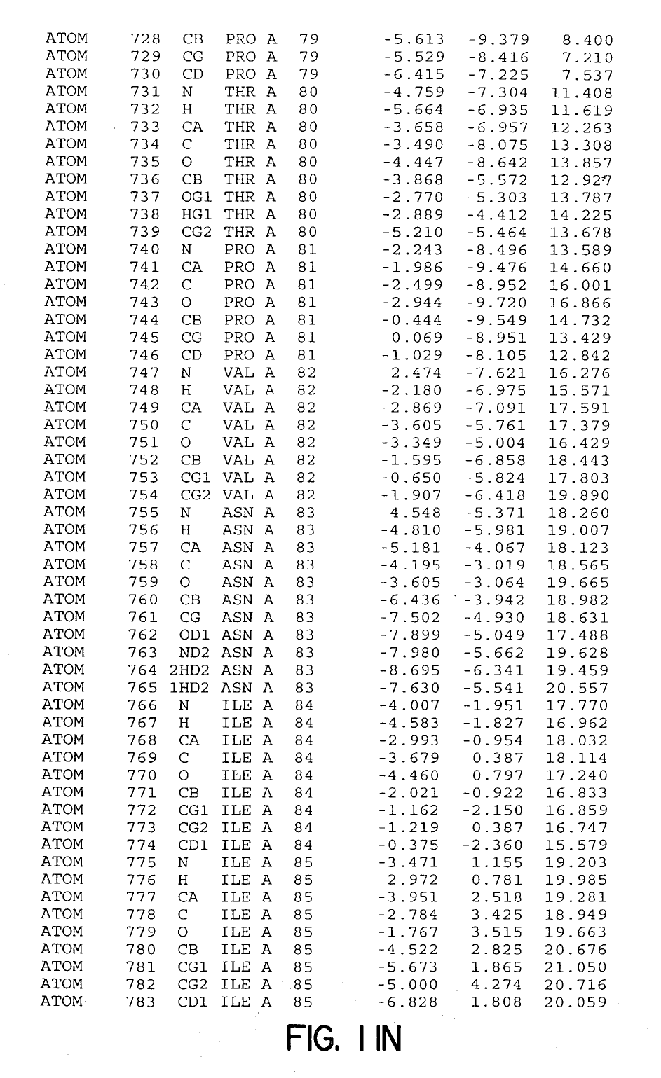

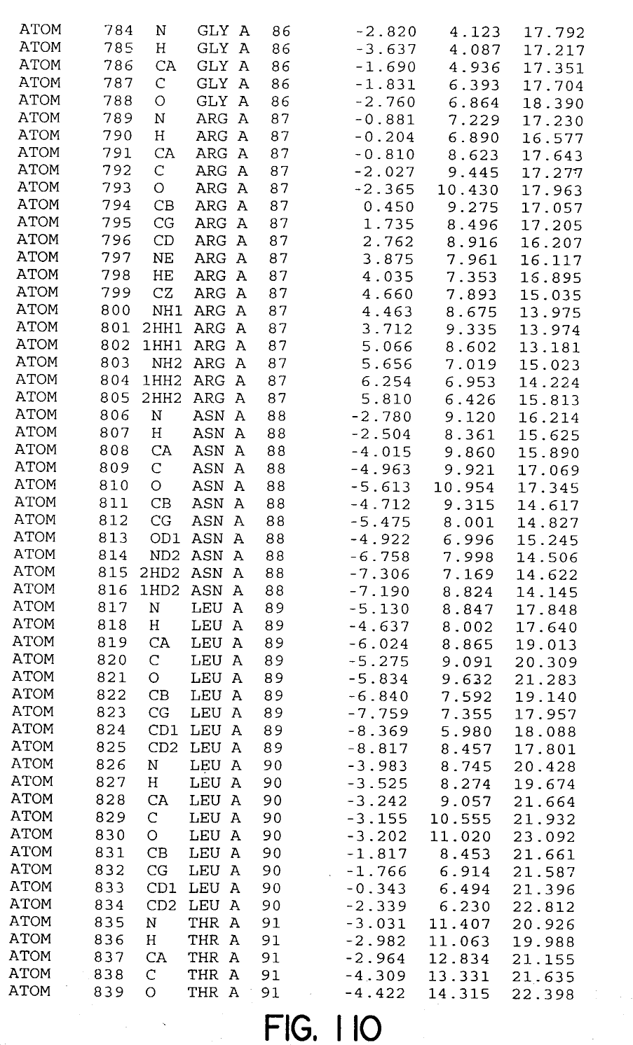

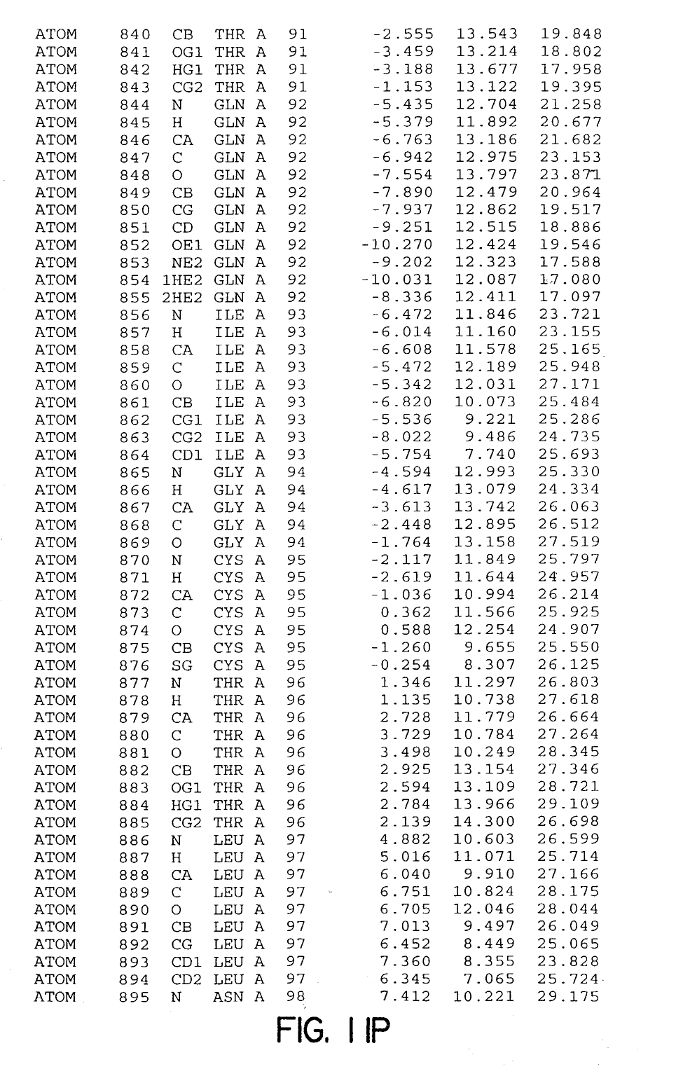

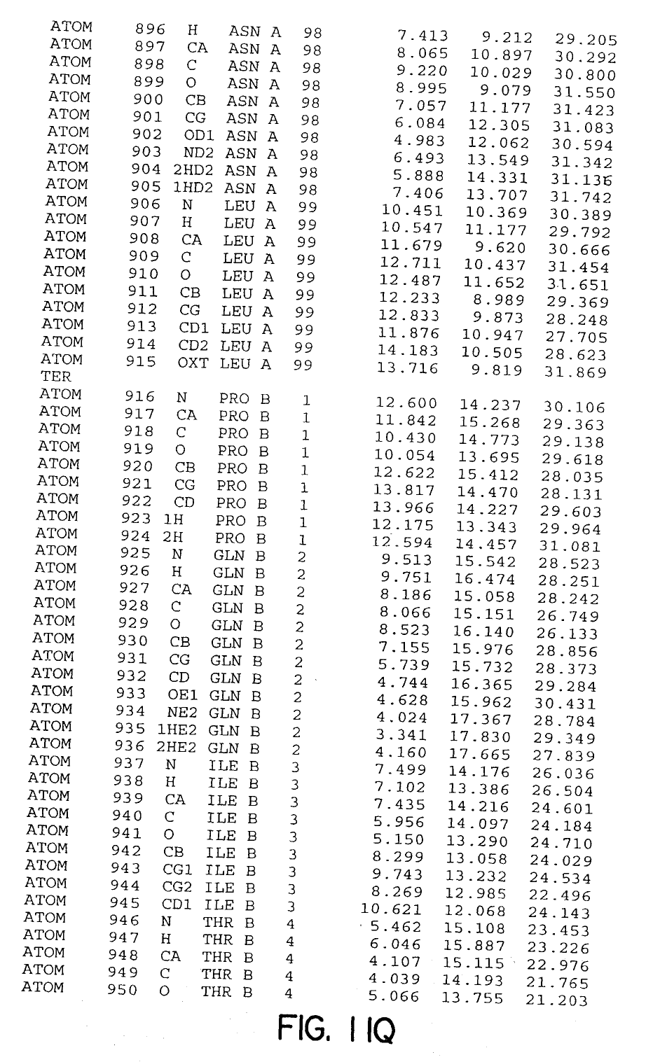

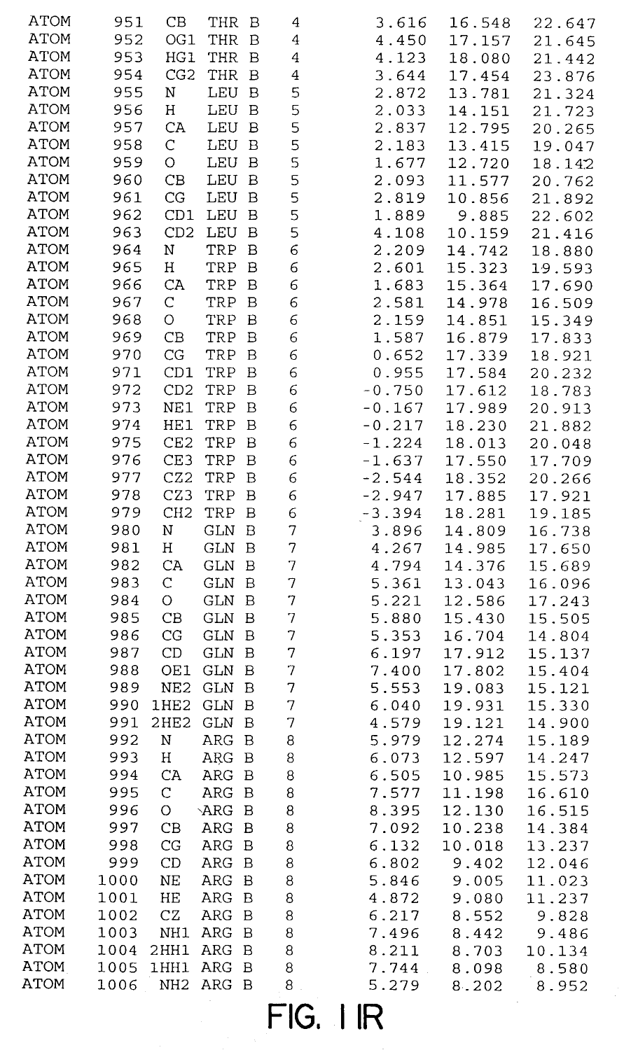

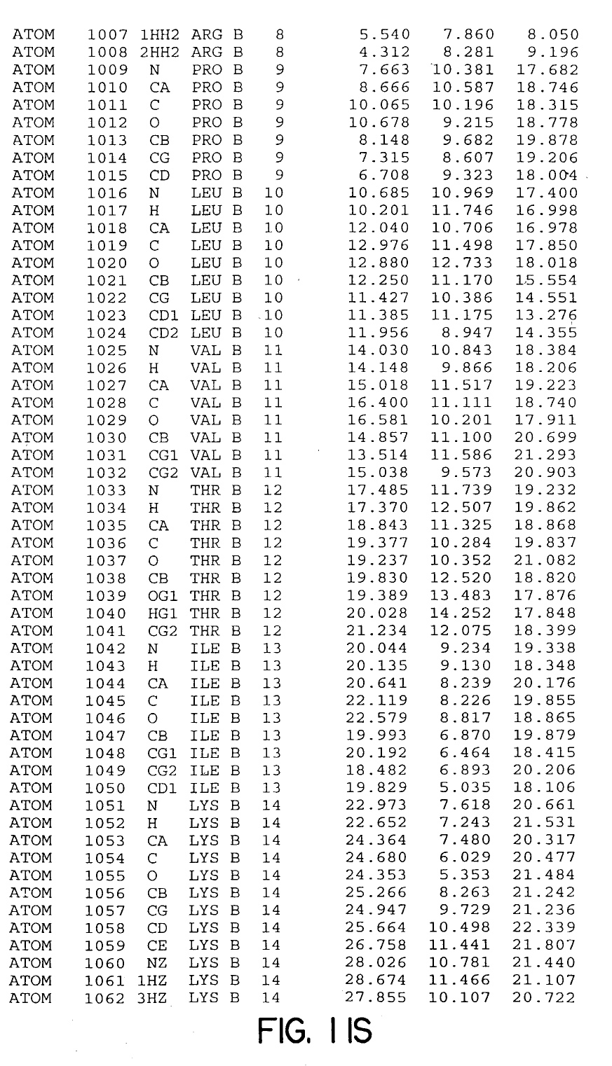

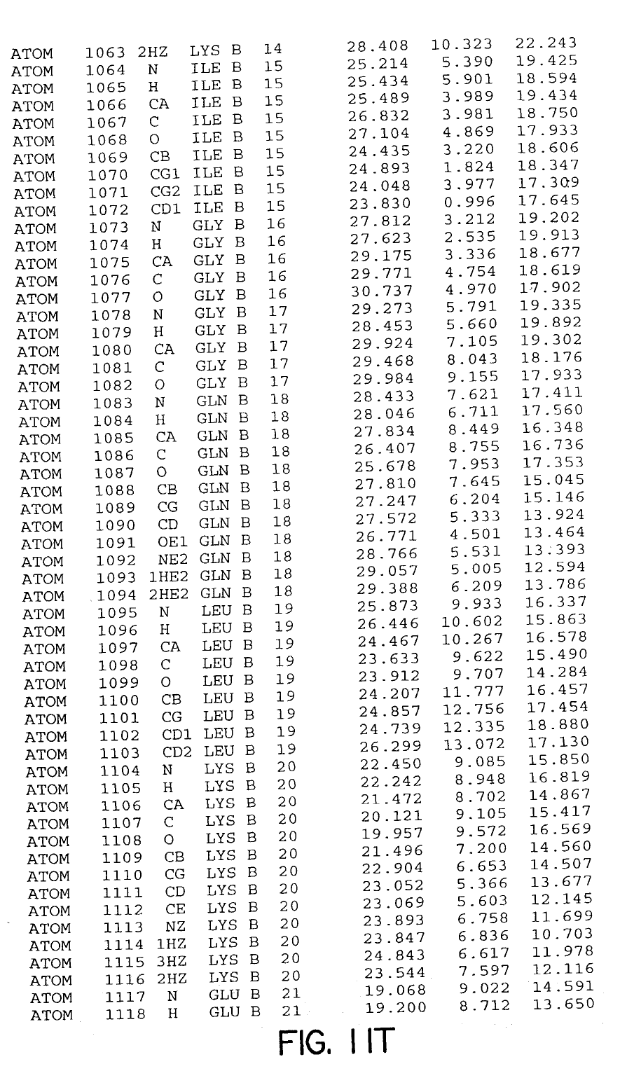

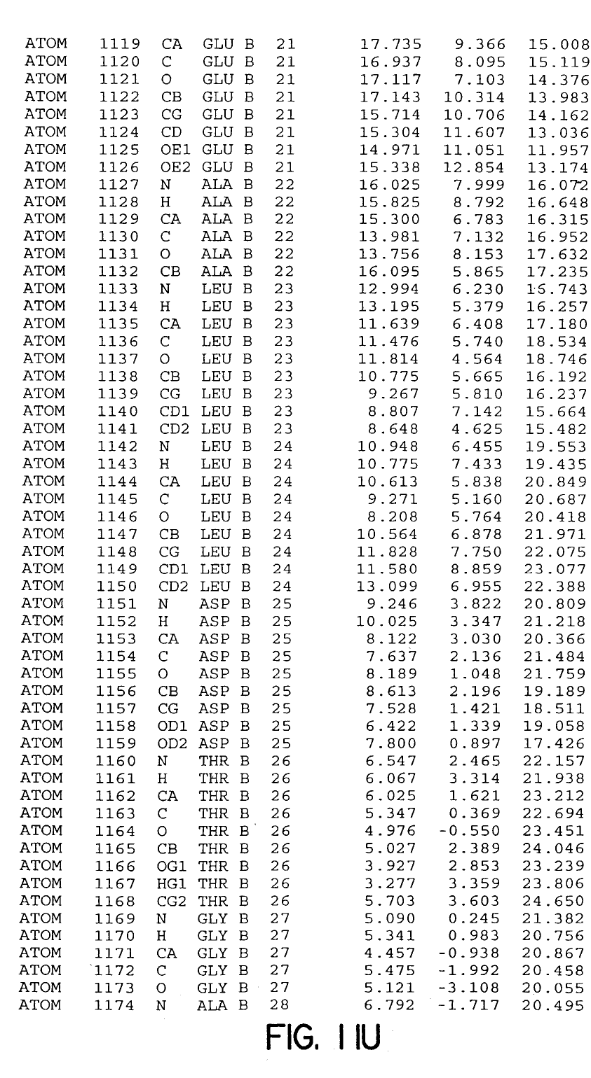

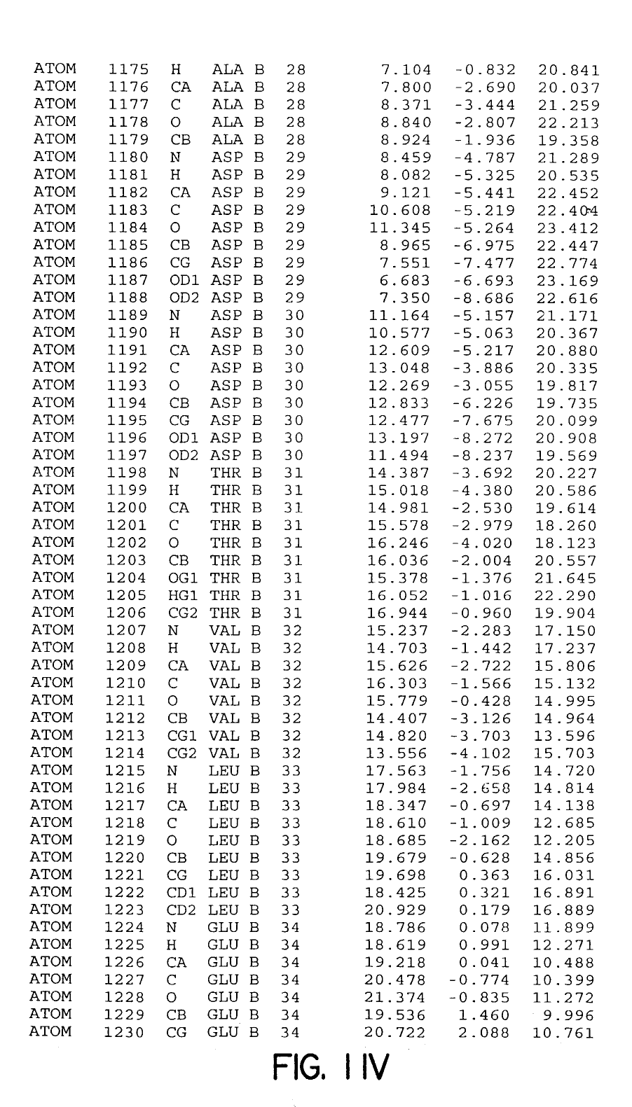

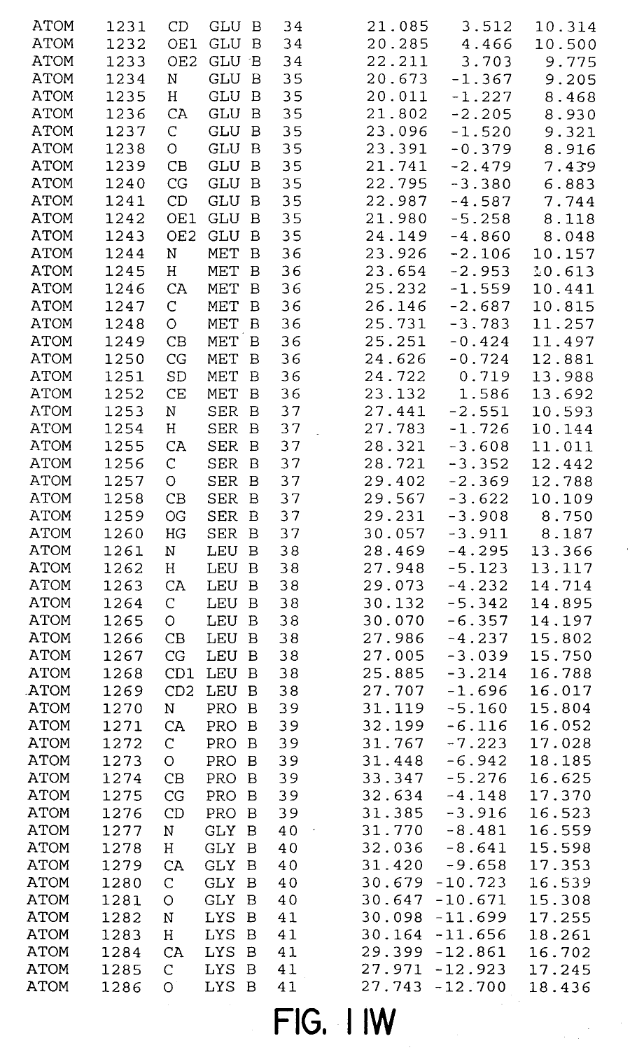

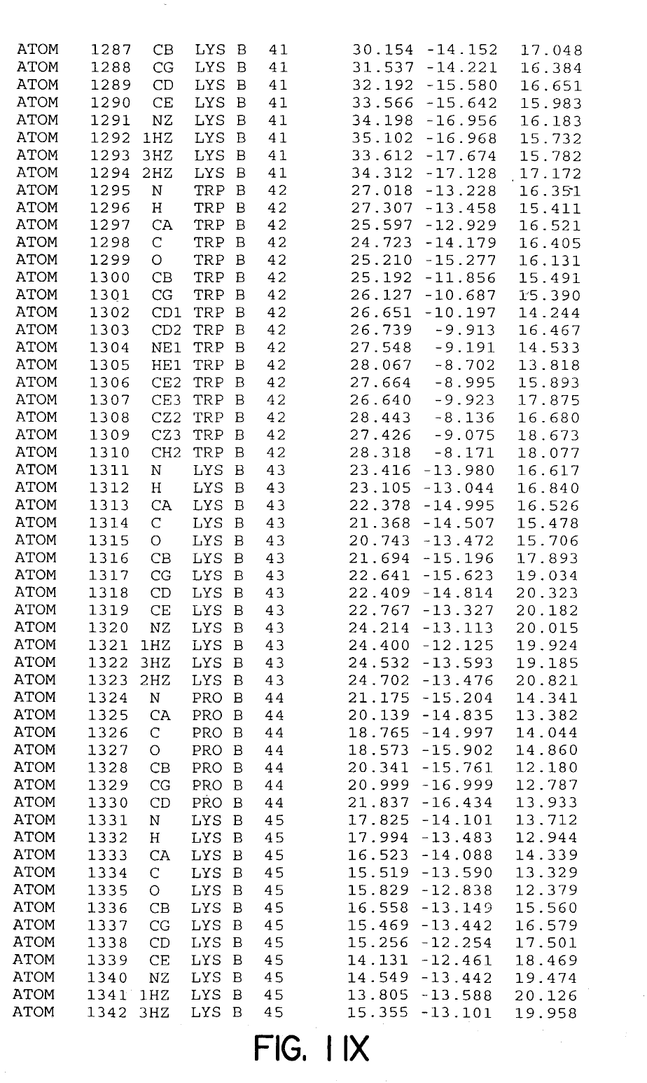

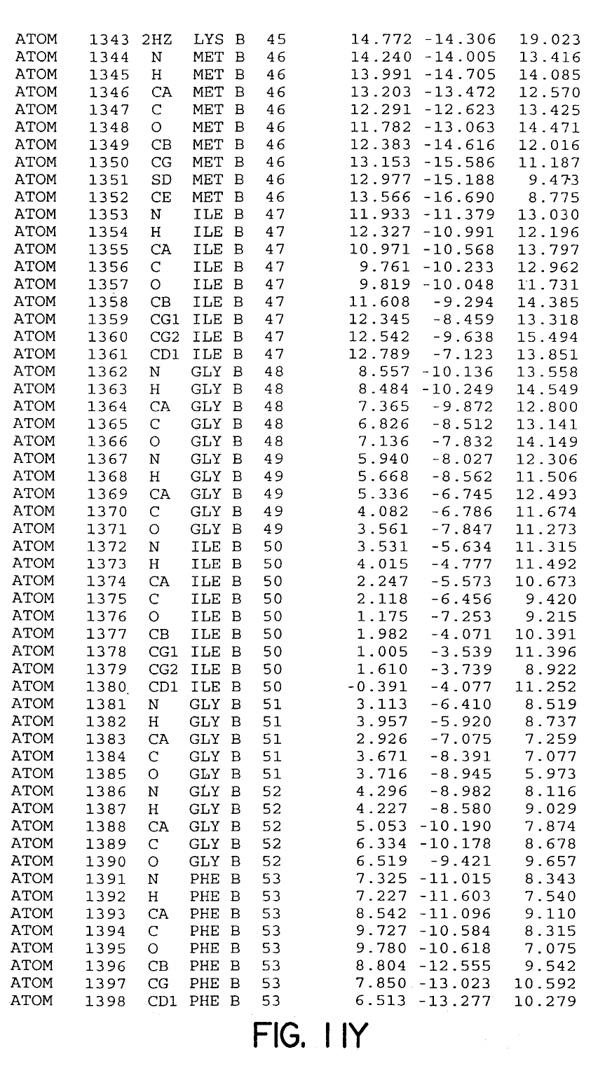

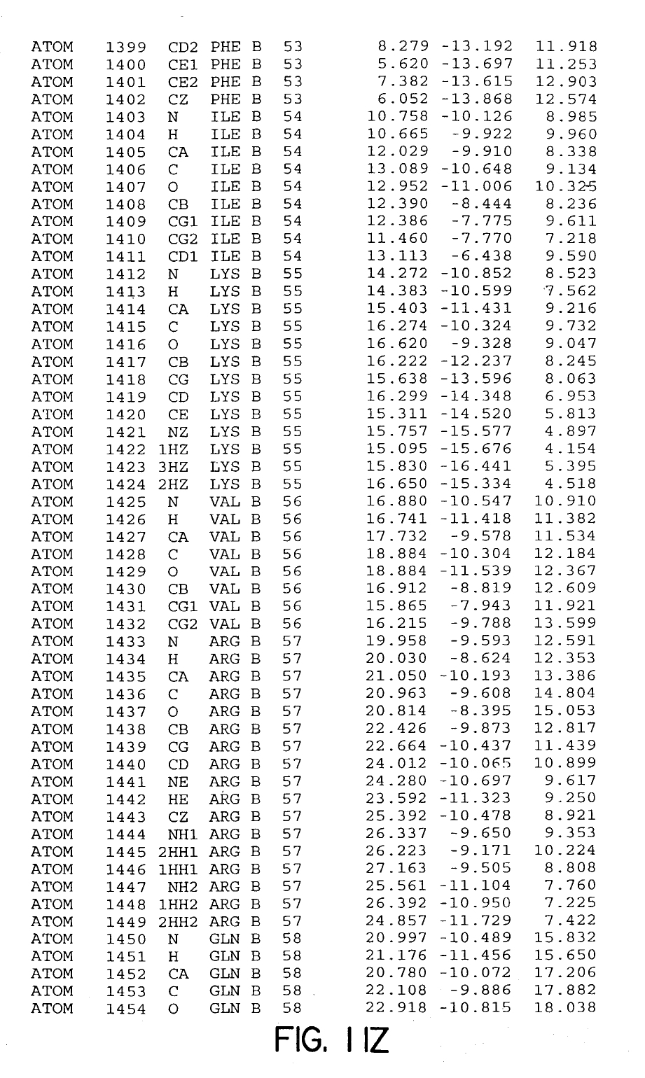

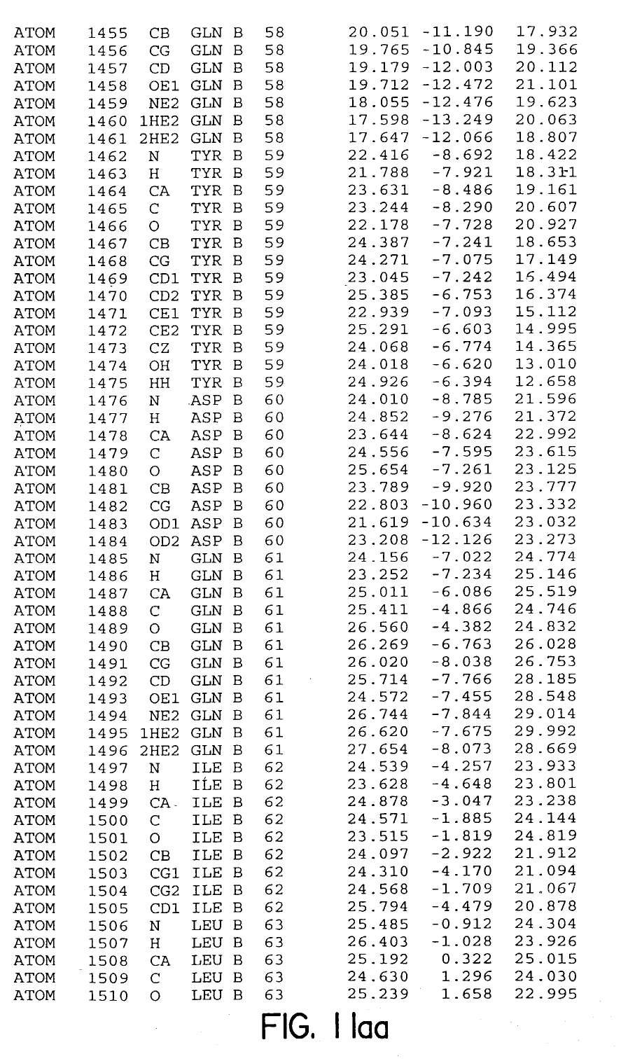

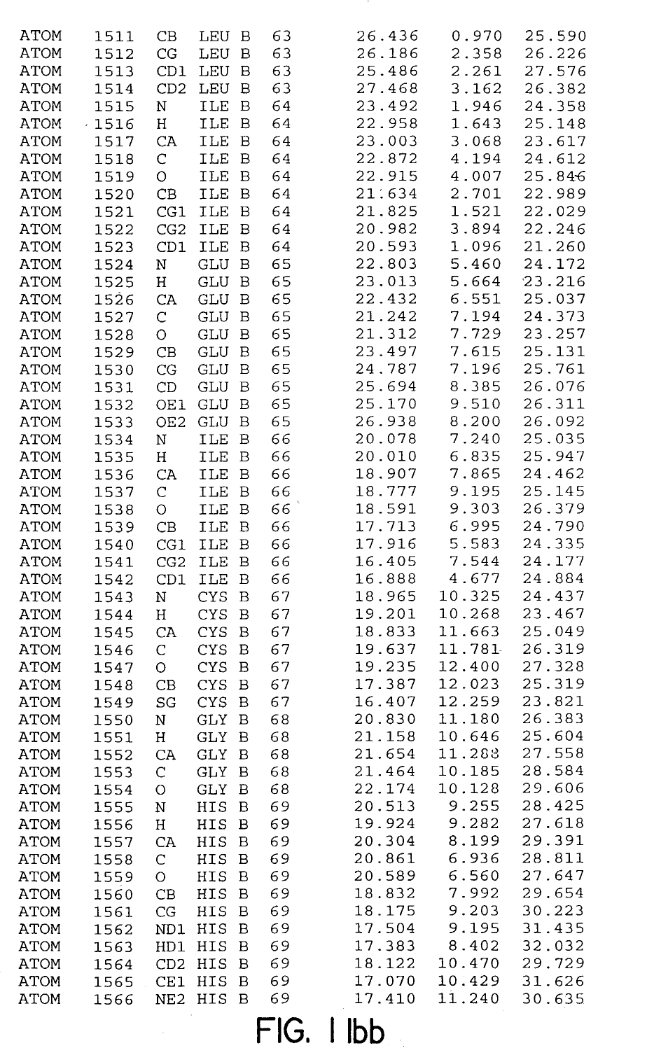

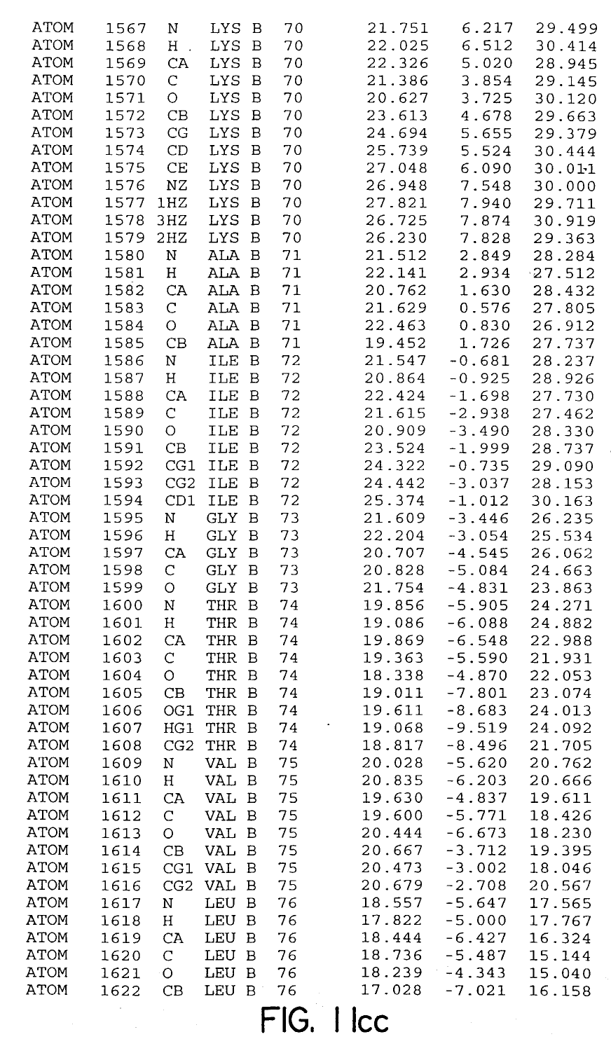

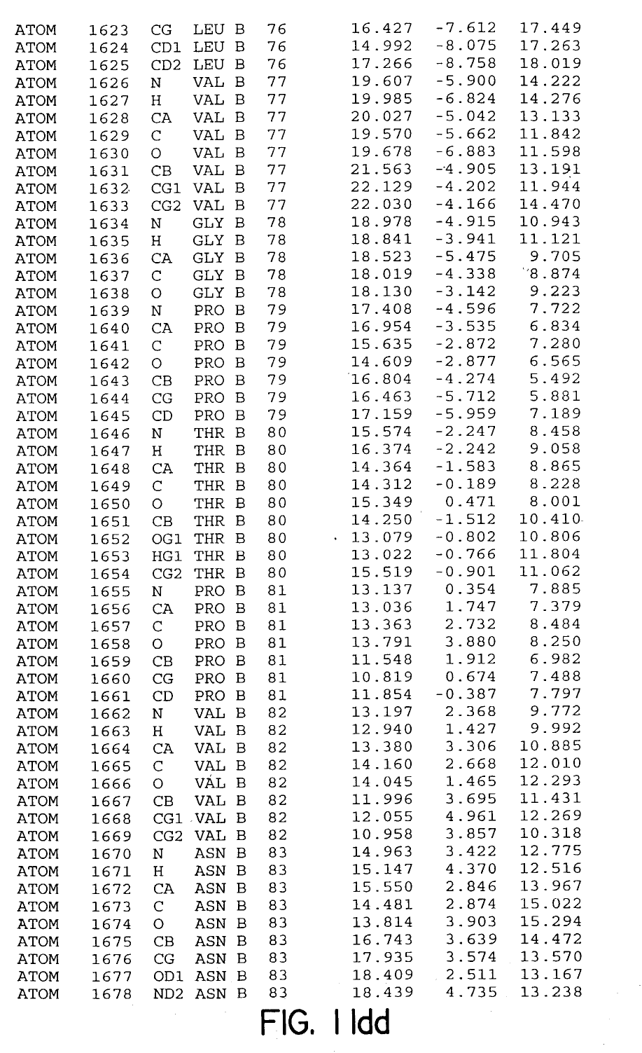

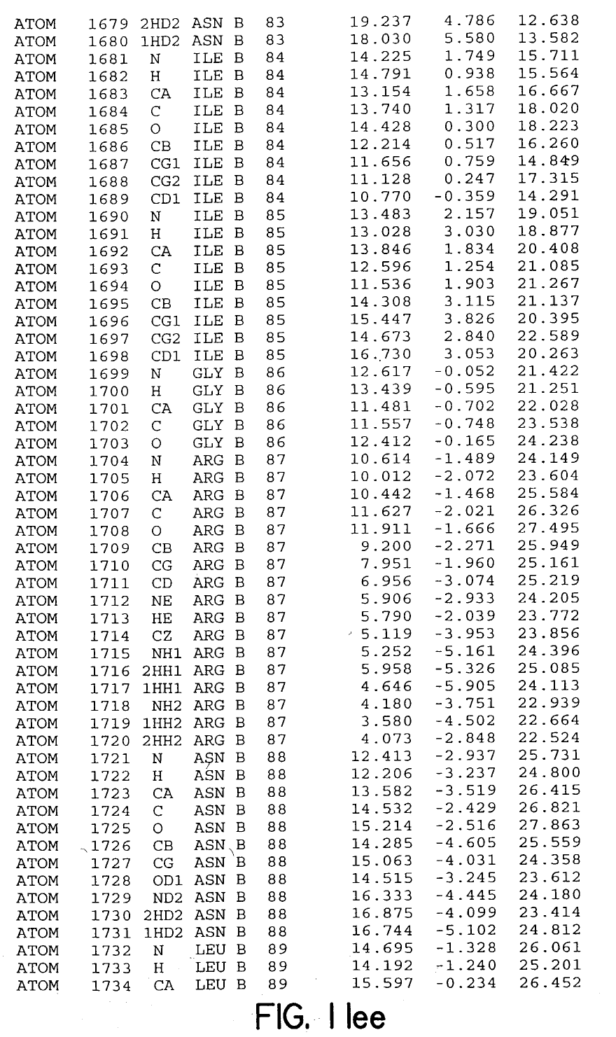

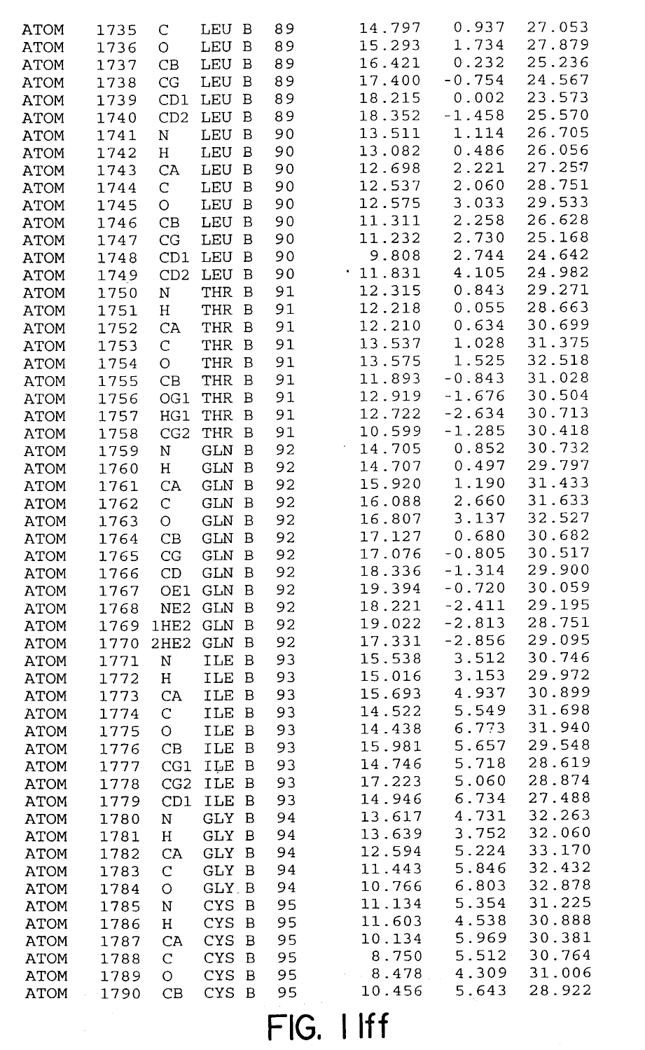

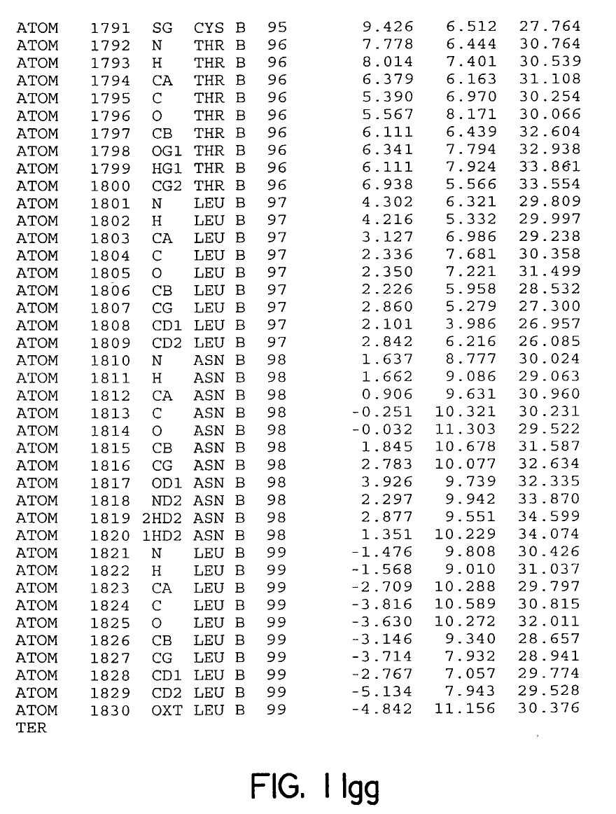

Databases that include 3-D structures for variant proteins encoded by the nucleic acids that contain polymorphisms are provided. These are generated after 3-D structural models are constructed for the protein structural variants, preferably for all of the protein structural variants, representing the genetic polymorphisms, by inputting the atomic coordinates into a structural polymorphism database, preferably a relational database, and optionally with associated structural and/or physical properties (e.g., phi/psi and side-chain angles and energetics), and other data, if available, including, but are not limited to, historical data, such as parental medical histories, and clinical data. The resulting database is used in structure-based drug design studies and for clinical analyses. FIG. 11 is a tabulation of the 3-D coordinates of a representative entry, an HIV protease, that is encoded by the DNA in one of SEQ ID Nos. 3-74 and 77-117, and that is an entry in an exemplary database that includes 3-D structures. Exemplary databases that contain the nucleic acids sequences and structures of all proteins encoded by SEQ ID Nos. 3-117 as well additional nucleic acids are provided herein and are described in the EXAMPLES.

A database is preferably interfaced to a molecular graphics package that includes 3-D visualization and structural analysis tools, to analyze similarities and variations in the protein structural variant models (see, copending U.S. application Ser. No. 09/531,995, which is published as International PCT application No. WO 00/57309, and is a continuation-in-part of U.S. application Ser. No. 09/272,814, filed Mar. 19, 1999). Briefly, International PCT application No. WO 00/57309 provides a database and interface for access to 3-D molecular structures and associated properties, which can be used to facilitate the design of potential new therapeutics. The interface also provides access to other structure-based drug discovery tools and to other databases, such as databases of chemical structures, including fine chemical or combinatorial libraries, for use in structure-focused high-throughput screening, as well as to a host of public domain databases and bioinformatics sites. The interface also provides access to other structure-based drug discovery tools and to other databases, such as databases of chemical structures, including fine chemical or combinatorial libraries, for use in structure-focused high-throughput screening, as well as to a host of public domain databases and bioinformatics sites. This interface can be modified as needed to adapt for use with a paritcular database.

A relational database that collects multiple data files relating to the same molecular structure in the same subdirectory and that provides an interface to access all of the collected files from the same structure using the same user interface program is also provided. The collected files include a variety of information and computer file formats, depending on the type of information to be conveyed to users of the database. In practice, a user communicates over a public network, such as the Internet, or over a controlled network, such as an internet, with a secure file server that controls access to the collected files, and the interface to the collected files is provided by a standard graphical user interface program that is widely available. In this way, a convenient means of searching molecular structure data for characteristics of interest is provided. Data searching, file viewing, and investigation of multiple representations of molecular structures from within a single viewing program can also be performed using the database and interface.

The data files can be those available over a wide network such as the Internet, and a suitable graphical user interface designed or obtained. Such interface is used for viewing the data files is a standard Internet web browser program, such as the web browser products by Netscape Communications, Inc. and Microsoft Corporation that are distributed free of charge. Such browser products readily import and provide views of files having a wide variety of formats that contain alphanumeric, video, and audio data. A security server is preferably located between the user browser program at a network client machine controls access to the database, which is housed at a file server connected to the security server. Before a user gains access to the database, the security server checks authorization for the individual user and then, if appropriate, permits downloading of appropriate data from the database file server. It is contemplated that the databases containing 3-D structures of proteins or portions thereof the exhibit polymorphism will be loaded.

Data for a molecular structure is loaded into the database by specifying the file pathnames for the various data files that contain the different types of data, including the different molecule views. Using a browser to view the data files permits various helper applications, called plug-ins, to smoothly and transparently accept the different file formats and provide views to the user. The various data files of the database are organized in accordance with the database design when they are loaded into the database and are managed by a relational database management program.

In addition to 3-D protein structures and associate primary sequences, as provided herein, the database can optionally contain associated biological or clinical data, such as drug resistance, side effects, efficacy, pharmacokinetics and other data, that correlate with or can be correlated the structural variants. This information will be used for correlating observed clinical effects to specific structural variants and for predicting clinical responses and outcomes based on a patient's structural variants, i.e., genetic polymorphisms.

Structural analysis tools are preferably integrated with the structural database for comparing and analyzing the resulting protein structural variant models. For example, the molecular graphics software package described in International PCT application No. WO 00/57309, includes structural analysis capability to measure the structural attributes of the model (distances, angles, etc.), to analyze sequences and secondary structures, to study physical properties such as hydrophobicity, electrostatic potential, and active or reactive sites in the protein, as well as to evaluate the quality of the structure (both conformationally and energetically).

Structures can also be compared by aligning them, such as by performing a least squares fitting of the x-, y- and z-coordinates of each of the structural variant models and superimposing the structures or any other alignment method or structural comparison method. For example, the structures of the variants can be clustered, or grouped together, based on structural similarity. This can save time over studying each structural variant independently because, where structures are considered to be similar enough that they are clustered together (e.g., if their structures can be superimposed within a specified tolerance), then only a representative structure, or perhaps an average structure or scaffold, which is derived as a composite of the individual structural variant models, can be used in further drug design studies.

Tools for database searching can also be included in the software package. These can be used to query the database for structural variant models having similar properties, such as molecular structure or sequence similarity. These tools are used, for example, to mine the database to identify variant models that are structurally similar (e.g. to find structures that overlap within a specified tolerance), and thus would be predicted to interact in the same way with potential drugs or exhibit the same clinical response. This information could be useful in understanding the structural or clinical effects of different genetic polymorphisms and could potentially save time and money by extending the results of previously performed clinical or computer-based drug design studies to predict the results of studies on similar structural variants that have not yet been performed.

1. Exemplary Databases

Databases containing data representative of the 3-D structure of structural variants encoded by a selected gene or genes or the 3-D structure of other polymorphic variants are provided. The selected genes can be genes of drug targets, such as receptors, and genes of infectious agents, such as the HIV protease or reverse transcriptase. Exemplary databases are presented in Example 5 which describes the construction, interface, use and applications of HIV PR and RT databases. These databases may be stored on any suitable medium and used in any suitable computer system. Systems and methods for generating, storing and processing databases are well known.

2. Computer Systems

Computer systems for processing the databases and computer systems containing the databases are provided. The processing that maintains the database and performs the methods and procedures using the databases may be performed on multiple computers, or may be performed by a single, integrated computer. For example, the computer through which data is added to the database may be separate from the computer through which the database is sorted or analyzed, or may be integrated with it. Each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically includes volatile semiconductor random access memory (RAM). Each computer preferably includes a program product reader that accepts a program product storage device from which the program product reader can read data (and to which it can optionally write data). The program product reader can include, for example, a disk drive, and the program product storage device can comprise removable storage media such as a magnetic floppy disk, an optical CD-ROM disc, a CD-R disc, a CD-RW disc, or a DVD data disc. If desired, computers can be connected so they can communicate with each other, and with other connected computers, over a network. Each computer can communicate with the other connected computers over the network through a network interface (see, e.g., Examples below) that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader, or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network. In the network method, the computer receives data including program steps into the memory through the network interface after network communication has been established over the network connection by well known methods that will be understood by those skilled in the art without further explanation.

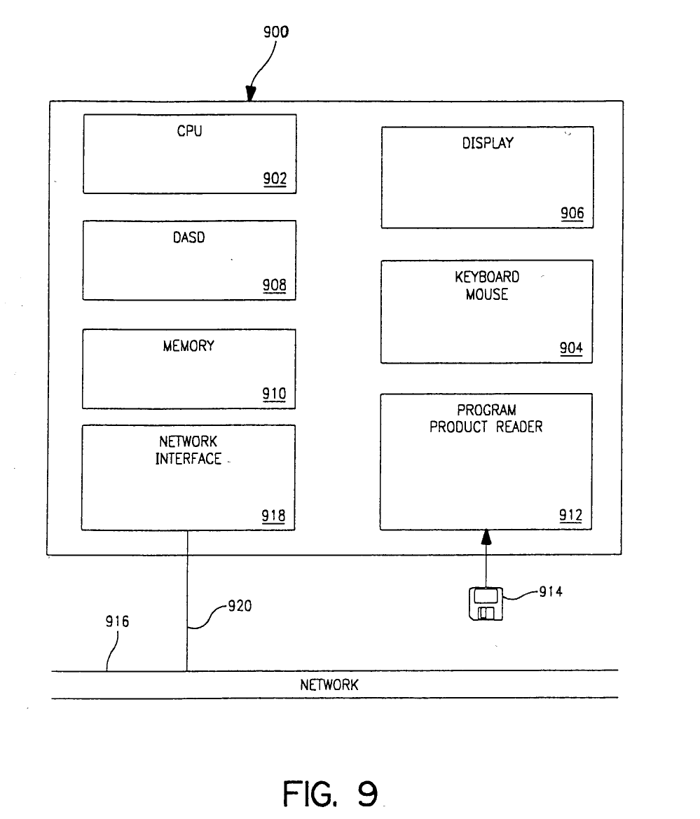

The computer that implements the client side processing, and the computer that implements the server side processing, or any other computer device of the system, may comprise any conventional computer suitable for implementing the functionality described herein. FIG. 9 is a block diagram of an exemplary computer device 900 such as might comprise any of the computing devices in the system. Each computer operates under control of a central processor unit (CPU) 902, such as an application specific integrated circuit (ASIC) from a number of vendors, or a “Pentium”-class microprocessor and associated integrated circuit chips, available from Intel Corporation of Santa Clara, Calif., USA. Commands and data can be input from a user control panel, remote control device, or a keyboard and mouse combination 904 and inputs and output can be viewed at a display 906. The display is typically a video monitor or flat panel display device.

The computer device 900 may comprise a personal computer or, in the case of a client machine, the computer device may comprise a Web appliance or other suitable Web-enabled device for viewing Web pages. In the case of a personal computer, the device 900 preferably includes a direct access storage device (DASD) 908, such as a fixed hard disk drive (HDD). The memory 910 typically comprises volatile semiconductor random access memory (RAM). If the computer device 900 is a personal computer, it preferably includes a program product reader 912 that accepts a program product storage device 914, from which the program product reader can read data (and to which it can optionally write data). The program product reader can comprise, for example, a disk drive, and the program product storage device can comprise removable storage media such as a floppy disk, an optical CD-ROM disc, a CD-R disc, a CD-RW disc, a DVD disk, or the like. Semiconductor memory devices for data storage and corresponding readers may also be used. The computer device 900 can communicate with the other connected computers over a network 916 (such as the Internet) through a network interface 918 that enables communication over a connection 920 between the network and the computer device.

The CPU 902 operates under control of programming steps that are temporarily stored in the memory 910 of the computer 900. When the programming steps are executed, the pertinent system component performs its functions. Thus, the programming steps implement the functionality of the system illustrated in FIG. 1. The programming steps can be received from the DASD 908, through the program product 914, or through the network connection 920, or can be incorporated into an ASIC as part of the production process for the computer device. If the computer device includes a storage drive 912, then it can receive a program product, read programming steps recorded thereon, and transfer the programming steps into the memory 910 for execution by the CPU 902. As noted above, the program product storage device can comprise any one of multiple removable media having recorded computer-readable instructions, including magnetic floppy disks, CD-ROM, and DVD storage discs. Other suitable program product storage devices can include magnetic tape and semiconductor memory chips. In this way, the processing steps necessary for operation in accord with the methods herein can be embodied on a program product.

Alternatively, the program steps can be received into the operating memory 910 over the network 916. In the network method, the computer receives data including program steps into the memory 910 through the network interface 918 after network communication has been established over the network connection 920 by well-known methods that will be understood by those skilled in the art without further explanation. The program steps are then executed by the CPU 902 to implement the processing of the system.

To implement the functionality described herein, it has been found that a suitable computer for performing database server tasks includes a “Pentium” level CPU having at least 128 MB of memory, 30 GB of disk storage, and 256 MB of disk swap space for files. A recommended configuration for computer performance would include, for example, a “Pentium III” processor at 700 MHz or faster, memory of 256 MB or greater, disk storage space of 50 GB or more, and swap space of 500 MB or more. A suitable configuration for performing user tasks as described above includes a “Pentium” level CPU having 128 MB memory, disk space of 240 MB with swap space of 256 MB, and an optional display circuit card supporting OpenGL and having 4 MB of memory. A recommended configuration includes, for example, a “Pentium III” processor at 500 MHz or faster, memory of 256 MB or greater, disk space of 500 MB or more, swap space of 500 MB or more, and an optional display card having 8 MB of memory or more, supporting resolution of 1024×768.

In a preferred embodiment, the software used in the computing system described above includes, for the server machine, operating system software such as “Windows NT Server 4.0” from Microsoft Corporation, with Service Pack 5, Version 1280 (10 Jun. 1999) or more recent, with database management server software such as, but are not limited to, “Oracle Server Standard Edition 8.1” from Oracle Corporation. The software used in a preferred embodiment of the user machine includes operating system software such as “Windows NT Workstation 4.0” from Microsoft Corporation, with Service Pack 5, version 1280 (10 Jun. 1999) or more recent, as well as “Oracle Client Standard Edition Version 8.1” or higher. The client machine will also be compliant with the “Java” programming language (Java Runtime Environment 1.2.2). As will be known to those skilled in the art, other configurations may be suitable, depending on the applications being used and the computer performance desired.

### E. COMPUTATIONAL PHENOTYPING

Also provided herein is a method designated computational phenotyping. Computational (also referred to herein as in silico phenotyping). This refers to the method in which a 3-D protein structure is generated from a given genotype and protein-drug binding analyses in silico (computationally) are performed in order to determine whether drug binding does (i.e. sensitive) or does not (i.e. resistant) take place. This type of analysis is contemplated to be performed for an individual patient or subject or groups thereof, such as ethnic groups, gender-based or age-based groups, particular species or groups thereof) to assess or select a drug for treatment of a particular disease or other such use, and is done to assess efficacy of a particular drug on a desired target, where the target exhibits polymorphisms. The following discussion and example, below, is with reference to HIV PR and RT, but it is understood that the methods and applications can be applied to any protein or gene product that exhibits polymorphic variation, and particularly to gene products that are drug targets.

Among the methods of computational phenotyping, there are three distinct methodologies that are clinically useful for determining either resistance or sensitivity to particular HIV-1 antiviral therapeutics. These are: genotyping, phenotyping, and virtual phenotyping. These methodologies are used to optimize the choice of therapeutics during the initiation of therapy, after drug failure, and/or during salvage therapy. Genotyping involves extracting the HIV viral RNA and amplifying all or part of the genes encoding the protease and reverse transcriptase proteins and sequencing them in order to assess the presence of resistance-associated mutations.

In phenotyping, the amplified sequences are instead sub-cloned into expression vectors and then tested for their replicative ability in vitro by transfecting them into cultured and/or established cell lines, such as, for example, human T cells, monocytes, macrophage, dendritic cells, Langerhans cells, hematopoeitic stem cells, HeLa, XC, Mm5MT, LTL, COS 7, NIH3T3, LTA, MCF-7, or other cells derived from human tissues and cells that which are the principal targets of viral infection in the presence or absence of antiviral drugs (see, e.g., U.S. Pat. No. 5,837,464; see, also EP 0852626; EP 1012334; and EP 0877937), Virtual phenotyping (ViroLogic, Inc.) is an interpretive service in which the phenotype of a specimen (i.e. of a plant, animal, pathogen, or human) is inferred from the specimen's genotype based upon an extensive correlative database of known genotypes and phenotypes. Such a correlative database must be updated constantly to maintain clinical accuracy.

Similar to virtual phenotyping, computational or in silico phenotyping infers phenotype based upon specimen genotype. Computational phenotyping is distinct from virtual phenotyping in that sensitivity or resistance to drugs is determined directly through protein-drug binding analysis performed in silico and not through correlation with a database of known genotypes and phenotypes. The advantage of computational phenotyping is that new resistance conferring mutations can be discovered rapidly and in “real time” without the need for phenotyping to train the genotype. Moreover, in silico phenotypes are not subject to error caused from compensatory mutations which may act synergistically or anti-synergistically with resistance-associated mutations to increase, decrease, or reverse specific drug resistances. Computational phenotyping will generate information that can, for example, be presented in a report that is marketed within the in vitro diagnostics industry as an adjunct test/service to help optimize therapy and assist physicians, farmers, academic institutions, government agencies, and industries with specimen treatment. Thus, a computer-based method for predicting clinical responses e.g. drug sensitivity or drug resistance in patients, plants, animals, pathogens, and microorganisms based on genetic polymorphisms is provided.

The genotypes used in the methods are obtained from any source, including, but are not limited to, from a plant, animal, pathogen, or mammal with the most preferred source being a mammal, particularly a human for whom a particular drug treatment is contemplated, and is the genotype of the drug target, such as, as exemplified herein, HIV RT or PR from a particular infected individual. Other exemplary drug targets are proteins, polypeptides, oligopeptides, including, but not limited to, a receptor, enzyme, hormone, and any such compound with which drugs or other ligands interact to bring about a biological response. For exemplification of this method, the protein considered is an enzyme, in particular HIV protease (PR) and reverse transcriptase (RT), which are therapeutic drug targets. Nucleic acid encoding the target from individual sample, such as blood sample or other body fluid sample from a mammal, such as a human patient, is sequenced, and the 3-D structure thereof determined. The drug of interest is computationally tested to assess whether it interacts with the sample.

The following examples are included for illustrative purposes only and are not intended to limit the scope of the invention.

### EXAMPLE 1

### Binding Correlations of Mutant Forms of Hepatitis C Virus (HCV) Protease with Different Inhibitors

This example provides the results of a theoretical study of NS3 protease complexes with two known peptide inhibitors (see SEQ ID Nos. 1 and 2; Ingallinella et al. ((1998) Biochemistry 37:8906-8914).

Introduction

During HCV replication, the final steps of processing are performed by a virally encoded chymotrypsin-like serine protease NS3. NS3 is an approximately 3000 amino acid protein that contains, from the amino terminus to the carboxy terminus, a nucleocapsid protein (C), envelope proteins (E1 and E2) and several non-structural proteins (NS1, 2, 3, 4a, 4b, 5a and 5b). NS3 is an approximately 68 kDa protein, encoded by approximately 1893 nucleotides of the HCV genome, and has two distinct domains: (a) a serine protease domain containing approximately 200 of the N-terminal amino acids; and (b) an RNA-dependent ATPase domain at the C-terminus of the protein. The NS3 protease is considered a member of the chymotrypsin family and is a serine protease that is responsible for proteolysis of the polypeptide (polyprotein) at the NS3/NS4a, NS4a/NS4b, NS4b/NS5a and NS5a/NS5b junctions responsible for generating four viral proteins during viral replication. This protease is inhibited by N-terminal cleavage products of substrate peptides. The NS3 protease, which is necessary for polypeptide processing and viral replication has been identified, cloned and expressed (see, e.g., U.S. Pat. No. 5,712,145).

Active NS3 forms a heterodimer with a polypeptide cofactor NS4A. The crystal structure of NS3 with and without the NS4A cofactor is known (see, e.g., Love et al. (1996) Cell 87:331-342; Habuka et al. (1997) Jikken Igaku 15:2308-2313; Yan et al. (1998) Protein Sci. 7:837-847, which provides the structure with NS4A).

The NS3 protease is a target for design of antiviral drugs. For example, a series of potent hexapeptide inhibitors of NS3 has been developed by optimization of the product inhibitors (Ingallinella et al. (1998) Biochemistry 37:8906-8914).

Analyses

Models of the complexes of NS3 with the two protease inhibitor peptides were obtained by flexible docking of the peptides into the active site of the crystal structure of NS3/4A, followed by evaluation of protein-peptide binding energies. The models were tested by in situ modification of the docked ligands. A qualitative agreement between the binding energies and inhibitor IC₅₀ values obtained from literature was found.

The peptides studied were:

| | | |
|---|---|---|
| | | SEQ |
| Sequence* | IC⁵⁰, nM | ID |

| | | |
|---|---|---|
| Ac-Asp¹-D-Glu²-Leu³-Ile⁴-Cha⁵-Cys⁶-COO- | 15 | 1 |
| Ac-Asp¹-L-Glu²-Leu³-Ile⁴-Cha⁵-Cys⁶-COO- | 60 | 2 |

Cha=β-cyclohexylalanine

In the modeling studies, it was assumed that:

the high-affinity inhibitory peptides 1 and 2 have a similar mode of binding to the active site of NS3;

the minimum binding pharmacophore includes the SH group of Cys⁶ and carboxyl groups of Asp¹, Glu² and Cys⁶; and

the side chains of residues 3, 4 and 5 may enhance binding by non-specific hydrophobic interaction with NS3.

Methods

Initial Structure of the NS3-Peptide Complex

The crystal structure of NS3 with a peptide cofactor NS4A was obtained from the arts (Kim et al. (1996) Cell 87:343) and was used in the studies with peptide inhibitors. The crystal structure of NS3/NS4A was regularized using molecular mechanics described herein. Initial NS3-NS4-peptide complexes were constructed by placing the peptides into the NS3 binding site expected by structural homology to by other serine proteases:

the C-terminal carboxyl was placed near the oxyanion-stabilizing site (residues 137-139);

the side chain of Cys⁶ was inserted into the hydrophobic cavity formed by L135, F154 and A157; and

the ε-amino group of K136 was placed in contact with the C-terminal carboxyl (see, Kim et al. (1996) Cell 87:343, Steinkuhler et al. (1998) Biochemistry 37:8899).

Monte Carlo Simulations

In order to optimize the complexes, Biased Based Probability Monte Carlo (BPMC) simulations (Abagyan et al. (1994) J. Mol. Biol. 235:983) were performed on the NS3-peptide complexes using the ICM program (commercially available from MolSoft, San Diego, Calif.) with ECEPP/3 force field and atomic solvation energies (Momany et al. (1975) J. Phys. Chem. 79:2361, Nemethy et al. (1992) J. Phys. Chem. 96:6472, Abagyan et al. (1997) Computer Simulations of Biomedical Systems: Theoretical and Experimental Applications, vol. 3, ESCOM Science Publishers BV, Leiden. The sampling method was BPMC with random change of one variable at a time. A Metropolis acceptance criterion was applied after energy minimization (quasi-Newton, up to 1000 steps). Simulations were performed at a temperature of 1000° K. The peptide translational and rotational degrees of freedom, all peptide torsion angles and X angles of the protein side-chains located within 7.0 Å of any peptide atom were varied during the BPMC simulations.

The energy function used in the MC simulations included:

ECEPP/3 terms for energy in vacuo (VDW (van der Waals), H-bond, electrostatic and torsion potentials);

distance dependent electrostatics with e₀=4.0; and

surface energy with atomic solvation parameters.

The total energies of the complexes were calculated including contributions from: ECEPP/3 VDW, H-bond, S-S bond and torsion terms; exact-boundary electrostatic energy with e₀=8.0; and side-chain entropies. Hydrophobic free energies were estimated as sA, where A is accessible surface area and s is a tension constant of 0.03 kcal/mol Å².

Strategy of the Flexible Monte Carlo Docking

The simulations proceeded with multiple, relatively short MC runs (2000-5000 generated structures). New docking cycles were started from the lowest-energy or other interesting structures found in previous runs. Structures saved during various MC runs were sorted by total energies and RMSD (root-mean-squared deviation), and compressed into a cumulative conformational stack. Binding energies were calculated for representative structures of each complex thus obtained. This strategy was more efficient than continuous long simulations because the variable torsion angles and distance constraints are defined for an initial structure and do not change during the MC run.

Binding Energies of the Peptide-Protein Complexes

For low-energy conformations found after several iterative BMPC cycles, peptide-protein binding energies were estimated using the equation:

E_{bind}=E₀+E_{compl}−Eₚₑₚₜ−Eₚᵣₒₜ,

where E_{compl} is the energy of the complex, Eₚₑₚₜ & Eₚᵣₒₜ are separate energies of the peptide and protein, respectively, and E₀ is an adjustable constant.

The binding energy function included: exact-boundary electrostatic free energy contributions; side-chain entropy; and surface tension hydrophobic free energy terms. (Zhou and Abagyan (1998) Folding Design 3:513, Schapira et al. (1999) J. Mol. Recognition 12:177). ECEPP/3 hydrogen-bonding terms were included with a weight of 0.5.

Results

Models of the NS3-Peptide Complexes

RMSD between pharmacophore atoms of peptides 1 and 2 were calculated for all pairs of BPMC structures. Two models of the NS3-peptide complexes were selected assuming (1) similar positions of pharmacophore groups of two peptides in the binding site (RMSD≦2.0 Å) and (2) low binding energy of the complexes (ΔE_{bind}<5.0 kcal/mol). Two models of the NS3-peptide complex were selected by visual inspection.

Characteristics of the binding sites for peptide inhibitors in two NS3-peptide complex models are summarized in Table 1.

TABLE 1

| | Peptide | NS3 residue, | Type of | Present for Peptide | |
|---|---|---|---|---|---|
| site | residue | group | interaction | Model 1 | Model 2 |
| P1 | Cys⁶COO⁻ | K136 NH₃ + | H-bond/el. | 1, 2 | 1, 2 |
| | | G137 NH | H-bond | 1, 2 | 2 |
| | | S139 OH | H-bond | 1, 2 | 2 |
| | Cys⁶ SH | L135, F154, A157 | hydroph | 1, 2 | 1, 2 |
| P2 | Cha⁵ | H57, R155, A156 | hydroph | 1, 2 | — |
| | | A157, V158 | hydroph | — | 2 |
| P3 | Ile⁴ | V132, S133 | hydroph | 1, 2 | 2 |
| | | V158, C159 | hydroph | — | 1 |
| P4 | Leu³ | Res. 157 to 160 | hydroph | 1, 2 | 2 |
| | | V132, S133 | hydroph | — | 1 |
| P5 | Glu² COO− | R161 guanidine | H-bond/el. | — | 1, 2 |
| P6 | Asp¹ COO− | R161 guanidine | H-bond/el. | 1, 2 | — |
| | | S133 OH | H-bond | — | 1, 2 |

Validation of the Models: Modifications of the Protein and Ligands in the Binding Site

In order to validate the proposed models, the K136M mutation and peptide modifications known from SAR (structure-activity relationship) studies were performed in low-energy structures of the NS3-peptide 2 complex.

Positions of the modified ligand and conformations of adjacent protein side chains were adjusted by energy minimization. Distance restraints were applied to keep the ligand near its initial position.

Changes in calculated binding energies upon modifications, ΔE_{bind}(calc), were compared to the values expected from ratios of inhibitory potencies, ΔE_{bind}(exp).

ΔE_{bind}(exp)=RT In(IC₅₀^{mod}/IC₅₀⁰),

where IC₅₀⁰ and IC₅₀^{mod} are inhibitory potencies of the parent and modified compounds.

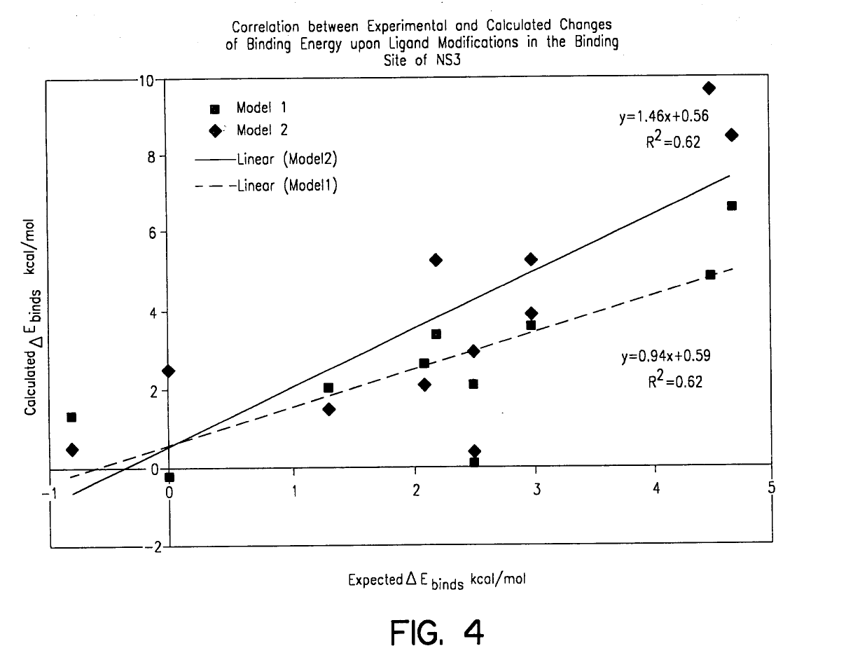

The correlation between experimental and calculated changes in binding energy upon ligand modifications in the binding site of NS3 is illustrated in FIG. 4.

Discussion

The two NS3-peptide complex models suggest a common binding pattern for the inhibitor P1 site (Cys⁶-OH) with the carboxyl group hydrogen-bonded to the oxyanion hole residues G137 and S139, and the Cys⁶ side chain embedded in a hydrophobic pocket formed by L135, F154 and A157.

This study confirms the possibility of hydrogen bonding between the C-terminal carboxyl and ε-amino group of K136 suggested by Steinkuhler et al. ((1998) Biochemistry 37:8899) based on the K136M mutation in NS3. Changes in calculated binding energies upon mutation are consistent with an 8-fold increase in K₁ of an inhibitor with a free carboxyl group and with the lack of an effect on binding when the peptide is amidated.

The models differ in binding of the negatively charged side chains in positions P5 and P6. The R161 guanidine interacts with a carboxyl group of Asp¹ and Glu² in Models 1 and 2, respectively. In Model 2, the Asp¹ carboxyl also interacts with the hydroxyl of S133.

The models are in agreement with SAR data for peptide inhibitors of NS3. Predicted changes in binding energy upon modification of the protein and peptides correlate reasonably well with the changes expected from IC⁵⁰ ratios. Standard deviations of ΔE_{bind}(calc)−ΔE_{bind}(exp) were 0.8 and 1.6 kcal/mol for Models 1 and 2, respectively, with correlation coefficients of 0.62. After the largest outlier was removed from each dataset, correlations improved to 0.81 and 0.76, respectively.

Conclusions

An effective iterative Biased Probability Monte Carlo protocol for the docking of flexible peptide ligands into a flexible protein active site has been developed. Two models of the complexes of HCV NS3 protease with potent peptide inhibitors were proposed based on the docking simulations and on evaluation of protein-ligand binding energies. The models were validated by in situ modifications of NS3-peptide complexes and by correlation of binding energies of modified complexes with those expected from experimental IC₅₀ values. Proposed models can be used for planning further mutagenesis studies of the HCV NS3 protease and the models can be used in the design of non-peptide inhibitors using structure-based drug design methodologies.

### EXAMPLE 2

### Lead Optimization by Receptor-Based free Energy Quantitative Structure Activity Relationships (QSARS) for Tumor Necrosis Factor (TNF) Receptor Antogonist Discovery

The goal of the modeling studies in this phase was to identify binding modes and complex structures of the compounds that bind to TNF receptor type I protein in order to guide the design of new compounds. An approach that relies on docking compounds to the receptor, evaluating free energy changes of binding of the docked structures, and comparing the calculated values with experimental inhibition constants K_{I} of the compounds was developed. The success of the calculations was assessed by evaluating the consistency of the calculated free energy changes of binding and the experimental K_{I}.

The difference in free energy changes of binding between two compounds with inhibition constants K_{I} and K_{I}′ can be calculated as,

ΔΔG=−kT InKᵢ′/K_{I}

where k and T are Boltzmann's constant and absolute temperature, respectively.

The 13 active compounds were studied. Their potencies, as measured by K_{I}, range from 0.1 to 30 μM, spanning about 3 kcal/mol in free energy. It was found that the calculated free energy changes of binding are highly consistent with the corresponding experimental values, with correlation coefficient 0.966 and difference less than 0.5 kcal/mol (see Table 2 and FIG. 4). The predicted binding modes and complex structures can thus be accepted with confidence.

To modify these compounds, important pharmacophore features on the surface of the receptor that are critical for binding of the compounds were identified. These features include a hydrophobic belt, a hydrophilic belt and 3 hydrogen bond donor sites. A few of potential hydrogen bonding sites, which are not used by the current compounds, were also derived, and can be used for designing more potent binders.

Graphics-guided redesign of the compounds was performed. The free energy calculation was used to predict the binding activity of each design. Fourteen new compounds were thus designed and binding activities were predicted. The chemical structures of the designed molecules, together with the binding modes of the lead compounds, were synthesized and shown to have high affinity for the target. Some of them exhibit a K_{I} in low-nanomolar range. Hence the method provided herein for modification of drugs for binding to calculated 3-D structures of a target protein resulted in redesigned drug candidates with enhanced affinity for the target.

This approach has advantages over the traditional x-ray crystallography method, which include the following:

(1) The binding modes are determined for a group of compounds instead of single compound; analysis of similarity and differences reveals rich information in binding mechanisms.

(2) The predictive power of the free energy calculation is very desirable for redesign of compounds.

(3) The correlation with the biochemical activities assures relevancy of the explored binding modes, while a structure given by x-ray crystallography may not necessarily be one related to the biological functions of the compound.

A comparison of calculated relative free energy changes of binding ΔΔΔ and experimental ΔΔG converted from inhibition constants K_{I} (all in kcal/mol) of the compounds (referenced by a code name) is presented in Table 2.

TABLE 2

| Compound | ΔΔA | ΔΔG |
|---|---|---|
| SBI-2030 | 0 | 0 |
| SBI-2002 | −0.97 | −1.25 |
| SBI-2005 | −0.72 | −1.14 |
| SBI-307 | −0.56 | −0.08 |
| SBI-2008 | −0.53 | −0.82 |
| SBI-2006 | −0.34 | −0.44 |
| SBI-306 | −0.07 | 0.40 |
| SBI-2000 | 0.29 | 0.27 |
| SBI-2001 | 0.72 | 1.12 |
| SBI-304 | 1.55 | 1.45 |
| SBI-308 | 1.70 | 1.78 |
| SBI-305 | 1.86 | 1.67 |
| SBI-2048 | 1.95 | 1.94 |

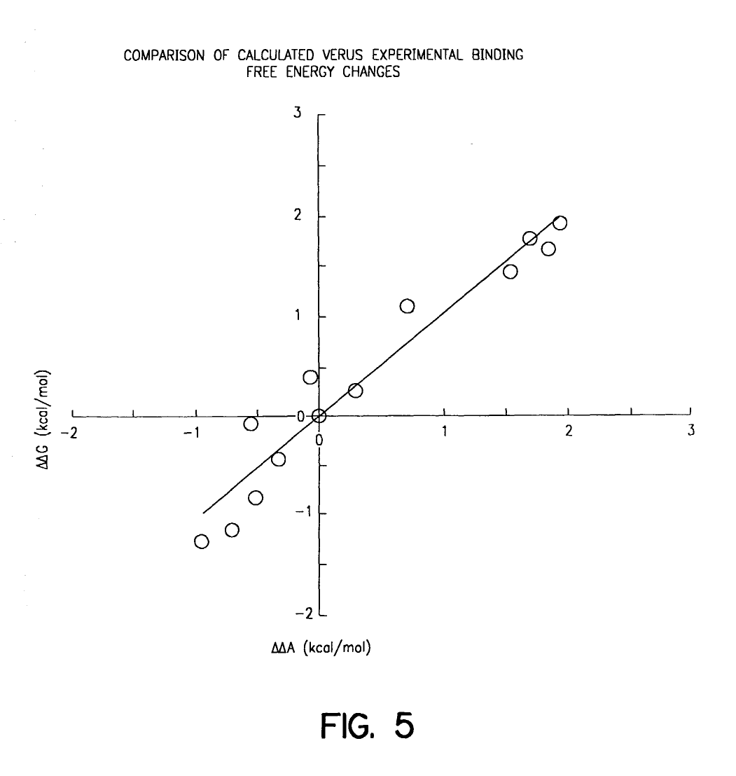

A comparison of calculated versus experimental binding free energy changes is given in FIG. 5.

### EXAMPLE 3

### HIV Protease Models for Drug Studies

Antiviral therapy for AIDS has focused on the discovery and design of inhibitors for two main enzyme targets of the HIV-1: reverse transcriptase (RT) and protease (PR). HIV RT is a heterodimer composed of p51 and p66 subunits. The p51 subunit is composed of the first 450 amino acids encoded by the RT gene and the p66 subunit is composed of all 560 amino acids of the RT gene. RT is responsible for RNA-dependent DNA polymerization, RNaseH activity, and DNA-dependent DNA polymerization.

HIV PR is a homodimer of two identical 99-amino acid chains. HIV PR is an aspartic proteinase that is responsible for the post-translational processing of the viral gag and gag-pol polyprotein gene products, which yields the structural proteins and enzymes of the viral particle (see, e.g., Erickson et al. (1996) Annu. Rev. Pharmacol. Toxicol. 36:545-571, Bouras et al. (1999) J. Med. Chem. 42:957-962). Despite several promising new anti-HIV agents, the clinical emergence of drug-resistant variants of HIV limits the long-term effectiveness of these drugs. Genetic analysis of the resistant forms of HIV has identified a number of critical mutations in the RT and PR genes. Moreover, structural analysis of inhibitor-enzyme complexes and mutational modeling studies can lead to a better understanding of how these drug-resistant mutations exert their effects at the structural and functional levels.

HIV-PR Inhibitor Computational Binding Studies

This example provides the results of a computational study on HIV PR. The 3-D protease structure was generated, docked with known viral inhibitors, and analyzed via free energy of binding studies described herein. A quantitative agreement between the calculated add experimental protease-drug binding energies was obtained. Moreover, a series of 3-D HIV PR models were analyzed to identify the invariant regions of the protease. These insights have implications for the design of new drugs and therapeutic strategies to combat AIDS drug resistance.

Optimization of 3D Structures

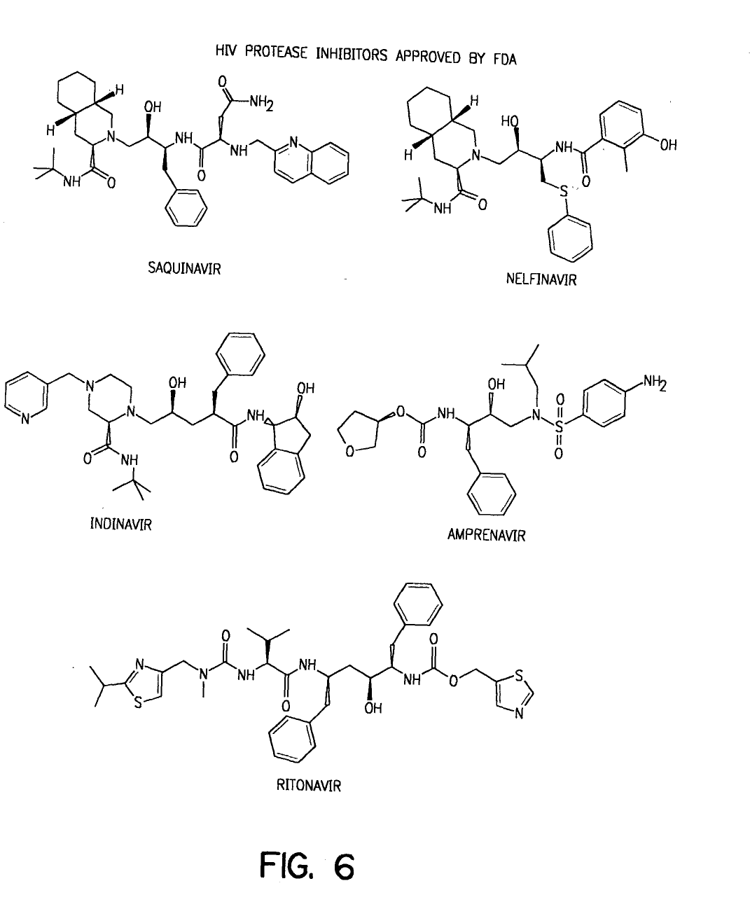

Five PR inhibitors approved by the FDA for clinical use were used: saquinavir, nelfinavir, indinavir, amprenavir, and ritonavir (FIG. 6). Initial 3-D structures for the wild-type HIV PR complexes with these FDA approved inhibitors were obtained from the Protein Data Bank and were then optimized using Monte Carlo (MC) simulations with an ECEPP/3 force field as described in Example 1. The energy function used in the MC simulations included: ECEPP/3 terms for energy in vacuo (van der Waals, H-bond, electrostatic and torsion potentials); distance dependent dielectrics with e₀=4.0; and surface free energy calculated using atomic solvation parameters ((Dudek et al. (1998) J. Computational Chem. 19:548-573, Wang et al. (1995) J. Mol. Biol. 253:473-492). Standard ECEPP charges were used for the protein residues. Lys, Arg, Glu, and Asp residues were charged. Charged and protonated states of Asp 125 (chain B) were considered as well. The inhibitors were docked into the active site of the protease, and the protein-drug complexes were energetically refined using the methods described in Example 1. Partial charges for the inhibitors were calculated with the Gasteiger-Marsili method implemented in SYBYL 6.5 (Tripos Assoc., Inc.). Different protonation states were examined for indinavir and amprenavir, but the other inhibitors were assumed to be electroneutral. Water molecules located within 7.0 Å from a ligand atom in the X-ray structure were retained in the model complex during optimization.

Calculation of Binding Energies

For low energy conformations found after several iterative BMPC cycles, protein-drug binding energies were estimated using the equation:

E_{bind}=E₀+E_{compl}−E_{ligand}−Eₚᵣₒₜ,

where E_{compl} is the energy of the complex, E_{ligand} & Eₚᵣₒₜ are energies of the ligand and protein when separated, and E₀ is an adjustable constant. The binding energies of the protein and ligand were calculated using the following energy function:

E=Eₑₗ+E_{vw}+E_{hb}+Eₛ,

where Eₑₗ is the exact-boundary electrostatic using e₀=8.0, Eₛ is the side-chain entropy term, and E_{vw} and E_{hb} are the ECEPP/3 van der Waals and hydrogen-bonding terms.

After the energies of the wild type PR-inhibitor complexes were calculated, mutation sites were introduced into the optimized X-ray structures or model complexes. The amino acid substitutions were followed by local optimization, using an ECEPP/3 force field, of protein side chains around the mutation sites via the energy minimization of substructures that included the ligand, water molecules within the sphere of radius 7.0 Å around the ligand, and protease residues within the sphere of radius 3-5 Å around the mutated residues. The energy of binding of the mutated complex was calculated based on the equation described herein. The difference in binding energy resulting from mutations (mut) of the wild-type (WT) protease were calculated using the following equation:

ΔE_{bind}(calculated)=E_{bind}(WT)−E_{bind}(mut).

This change in binding energy was compared to data from experimental (exptl) studies (Gulnik et al. (1995) Biochemistry 35:9282-9287, Klabe et al. (1998) Biochemistry 37:8735-8742, Pazhanisami et al. (1996) J. Biol. Chem. 271:17979-17985, Jacobsen et al. (1995) Virology 206:527-534, Maschera et al. (1996) J. Biol. Chem. 271:33231-33235) based on the equation:

ΔE_{bind}(exptl)=RTIn(K_{I}mut/K,wt).

Plots of ΔE_{bind}(calculated) vs. ΔE_{bind}(exptl) were generated, and the results, summarized in Table 3, show a strong correlation between the calculated binding energies and the experimentally determined binding energies for the PR-inhibitor complexes. For example, the correlation coefficient R for PR-ritonavir and PR-amprenavir is 0.9, where R=1 denotes congruency between the computationally calculated and experimentally determined binding energy data. These correlation data validate the computational protocol and calculations described herein as a method for predicting protein-drug binding or protein-drug resistance (i.e. non-binding). The evaluation of changes in binding energy of protein-drug complexes upon protein sequence variations can be used as a possible descriptor and, thus, can be used to predict the efficacy of drugs on proteins resulting polymorphisms in genes. Moreover, the analysis of the free energy of binding in complexes between the protein models that are produced by the method set forth in this example and drugs that have been designed or modified is a good predictive tool for drug designers.

TABLE 3
Correlation between Experimental and Calculated Binding Energies
for HIV Protease Inhibitors

| HIV | X-ray | No of exptl. | Correlation | Correlation |
|---|---|---|---|---|
| PRInhibitor | Complex ID | data points | coefficient R | S.D., kcal/mol |
| Saquinavir | 1HXB | 18 | 0.84 | 0.68 |
| Indinavir | 1HSG | 17 | 0.79 | 0.80 |
| Ritonavir | 1HXW | 12 | 0.90 | 0.72 |
| Amprenavir | 1HPV | 15 | 0.90 | 0.54 |

| | | |
|---|---|---|
| Nelfinavir | 1OHR | Insufficient data |

Identification of Structural Invariant Regions of HIV Protease

Clinical effectiveness of HIV PR inhibitors is limited by the rapid emergence of drug-resistant mutations. Resistant PR variants first occur by the mutation of amino acids close to or in and around the drug binding site, which are then accompanied by compensatory mutations of more distant amino acids. The identification of highly conserved, structural invariant regions of a PR would provide new potential targets and thus lead to the development of therapeutics having greater clinical efficacy than those drugs commonly employed to treat HIV.

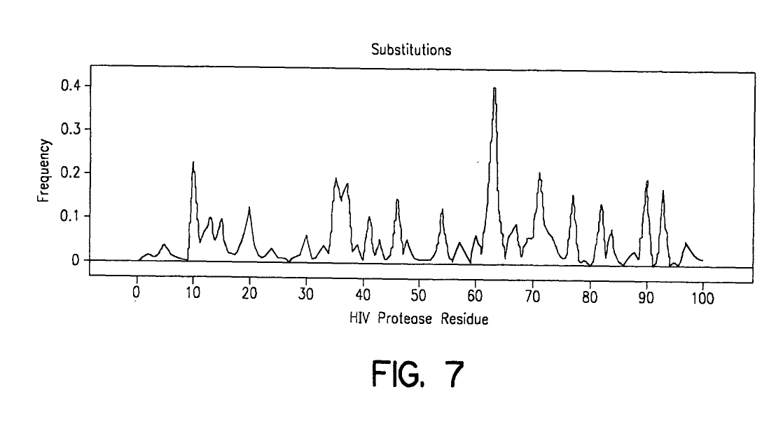

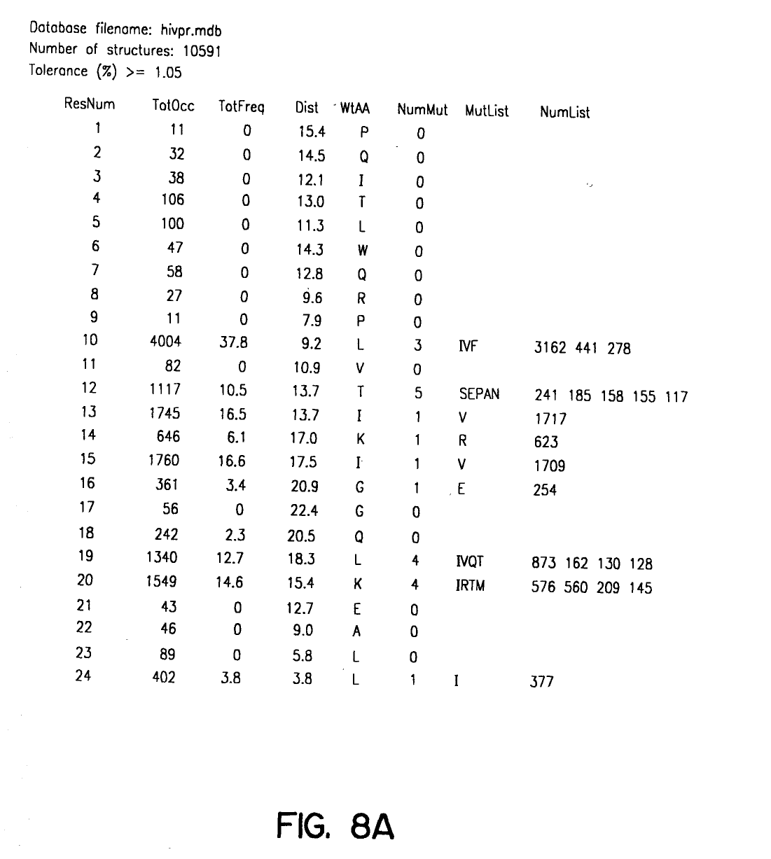

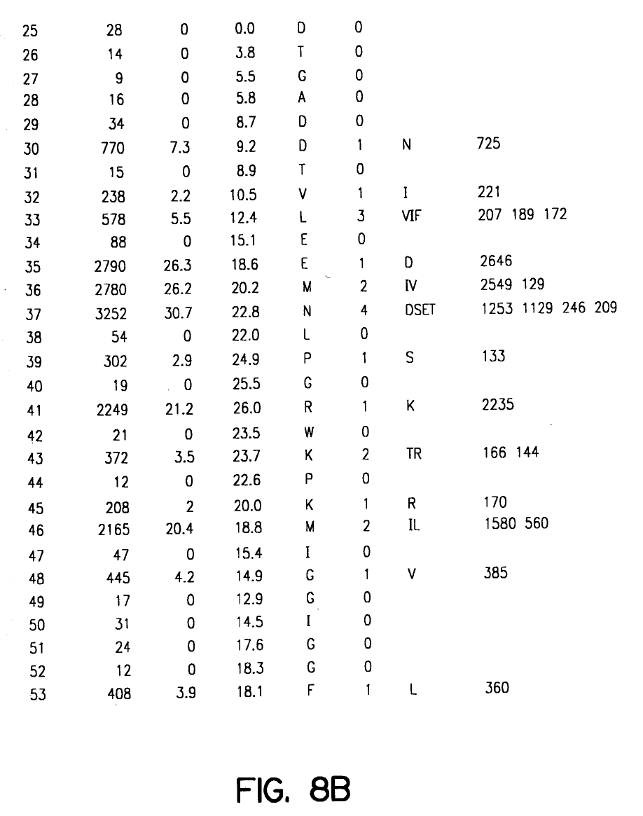

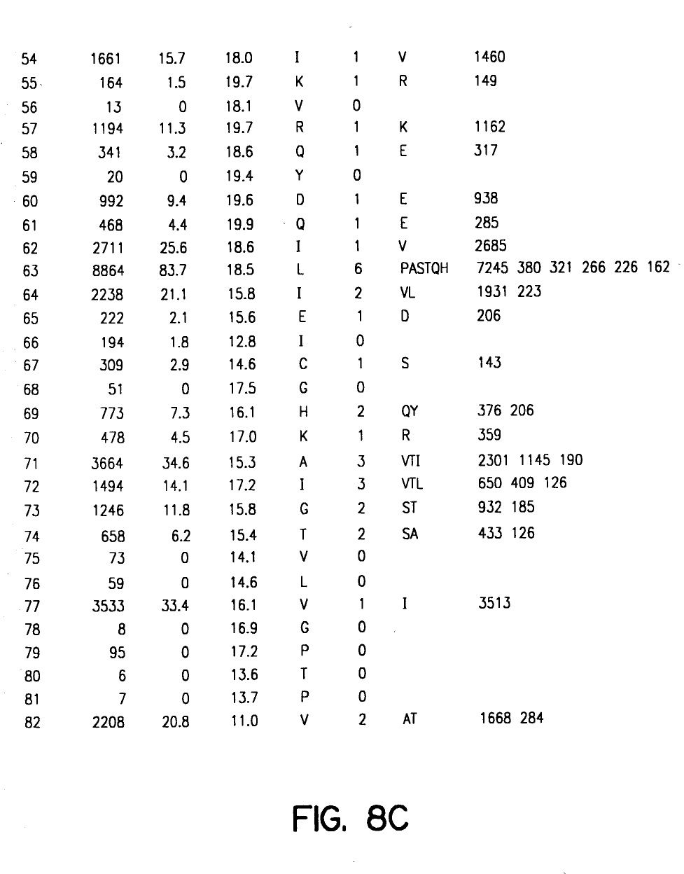

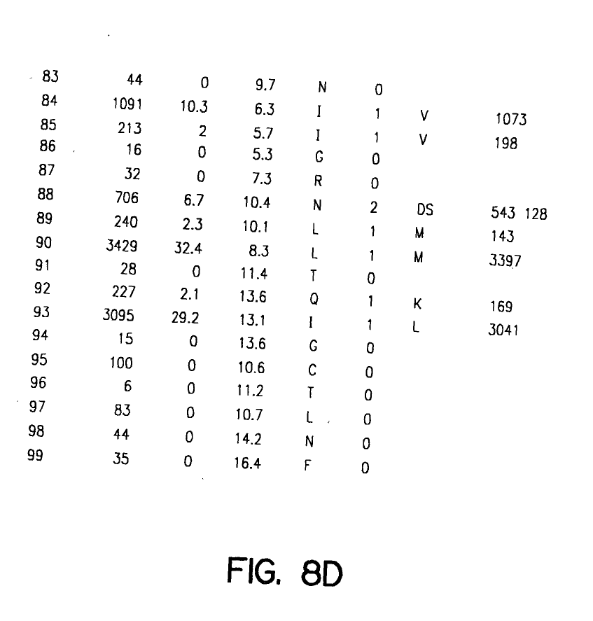

The protein sequences of HIV protease were obtained from GenBank and from the blood samples of patients using standard isolation and sequencing techniques well known in the arts. The protein sequences were modeled into 3-D structures using the computational protocol described in Example 1. The protease sequences were aligned, and the frequency of mutation, regardless of type, was determined at each amino acid position and plotted in FIG. 7, where the frequency of mutation in this set of HIV-1 Protease sequences varied from 0 to 40%. Sequence alignment also revealed how many different types of amino acids could be substituted in any specific residue, yielding the tolerance of each residue to substitutions of different types. The data showing the frequency of mutation of each residue out of PR sequences, the types of mutations, and the distance of the mutating residue from the active site (Asp 28) are shown in FIG. 8. This information, sequences obtained from 10591 different genotypes, was used to identify invariant and/or highly conserved regions of PR and to map these regions to a 3-D structure for the purpose of identifying new potential regions on the protein as targets for therapeutic intervention. These invariant regions include, but are not limited to, residues 1-9, 25-29, 49-52, 78-81, and 94-99, where residue 1 is an aliphatic amino acid, more preferably proline; residue 2 is a hydrophilic amino acid, more preferably glutamine; residue 3 is an aliphatic amino acid, more preferably isoleucine; residue 4 is a hydrophilic amino acid, more preferably threonine; residue 5 is a hydrophobic amino acid, more preferably leucine; residue 6 is an aromatic amino acid, more preferably tryptophan; residue 7 is a hydrophilic amino acid, more preferably glutamine; residue 8 basic amino acid, more preferably arginine; residue 9 is an aliphatic amino acid, more preferably proline; residue 25 is a hydrophilic amino acid, more preferably aspartic acid; residue 26 is a hydrophilic amino acid, more preferably threonine; residue 27 is an aliphatic amino acid, more preferably glycine; residue 28 is an aliphatic amino acid, more preferably alanine; residue 29 is an acidic amino acid, more preferably aspartic acid; residue 49 is an aliphatic amino acid, more preferably glycine; residue 50 is a hydrophobic amino acid, more preferably isoleucine; residue 51 is an aliphatic amino acid, more preferably glycine; residue 52 is an aliphatic amino acid, more preferably glycine; residue 78 is an aliphatic amino acid, more preferably glycine; residue 79 is an aliphatic amino acid, more preferably proline; residue 80 is a hydrophilic amino acid, more preferably threonine; residue 81 is an aliphatic amino acid, more preferably proline; residue 94 is an aliphatic amino acid, more preferably glycine; residue 95 is a thio-containing amino acid, more preferably cysteine; residue 96 is hydrophilic amino acid, more preferably threonine; residue 97 is hydrophobic amino acid, more preferably leucine; residue 98 is hydrophilic amino acid, more preferably asparagine; and residue 99 is an aromatic amino acid, more preferably phenylalanine. These invariant regions can subsequently be used to assist in the design drugs or therapeutic agents which bind to the invariant regions and disrupt the activity of the protease with greater efficacy than drugs commonly used to treat HIV and where the free energy of binding between said drug or therapeutic agent and the structural invariant region is evaluated as described herein. The methods described in this example can also be applied to HIV RT and to any protein of interest that exhibits polymorphisms.

### EXAMPLE 4

Computational Phenotyping of HIV-1 Protease and Reverse Transcriptase Computational or in silico phenotyping is performed to assess phenotypic properties of a protein. This example demonstrates application of this method to HIV-1 protease and reverse transcriptase to test whether the efficacy of various protease inhibitors for an HIV patient.

To practice this method 3-D structures of HIV-1 protease and reverse transcriptase based upon the nucleic acid isolated from HIV from a patient are generated. Protein-drug binding analysis in silico in order to determine whether drug binding does (i.e. sensitivity) or does not (i.e. resistance) take place.

Sequencing of HIV-1 Protease and Reverse Transcriptase is performed on HIV-1 cDNA following extraction, reverse transcription, and PCR amplification of viral RNA obtained from patient specimens, such as blood samples or other body fluid or tissue samples. Methods for the extraction, reverse transcription, and PCR amplification of viral RNA are well known in the art. For each sequence, a computer-generated 3-D structure of the protein is modeled and then docked with antiviral drugs in silico using methods described in Example 1 and elsewhere herein to analyze protein-drug interactions. Antiviral drugs that can be tested include, but are not limited to, saquinavir, indinavir, ritonavir, amprenavir, and nelfinavir for HIV protease; zidovudine, lamivudine, stavudine, zalcitabine, didanosine, abacavir, adefovir, delavirdine, nevirapine, and efavirenz for HIV reverse transcriptase; and any FDA-approved or non-FDA approved antiviral drug. From these protein-drug interaction studies, relative drug resistance or sensitivity is inferred by calculating and evaluating the free energy of binding in low energy conformations of complexes between the variant protease structure and docked antiviral drug or variant reverse transcriptase structure and docked antiviral drug, using the methods described in Examples 1 and 3 and elsewhere herein.

The results of the computational phenotyping procedure can be presented as a patient report that states whether a drug or drugs are sensitive or resistant to the RT or PR obtained from the patient. Such a patient report assists physicians in selecting appropriate drugs for HIV patients. It also is useful for the in vitro diagnostics industry in an adjunct test/service capacity to help optimize antiviral therapy.

### EXAMPLE 5

### HIV Protease and Reverse Transcriptase Databases

Exemplary databases of the 3-D protein structures of polymorphic variants are described in this example. The HIV PR and RT databases are a comprehensive collection of 3-D polymorphic structural data along with related information, including nucleic acids encoding all or a portion of the protein. These data provide a means to understand differences in the interactions between a drug or drugs and the structural variations of the drug targets. This example describes the creation, interface for, and use of structural variant databases of HIV protease and reverse transcriptase polymorphic variants.

Construction of Databases

To implement the RT or HIV database described herein, suitable computer for performing database server tasks includes a “Pentium” level CPU having at least 128 MB of memory, 30 GB of disk storage, and 256 MB of disk swap space for files. A recommended configuration for better computer performance would include, for example, a “Pentium III” processor at 700 MHz or faster, memory of 256 MB or greater, disk storage space of 50 GB or more, and swap space of 500 MB or more. A suitable configuration for performing user tasks as described above includes a “Pentium” level CPU having 128 MB memory, disk space of 240 MB with swap space of 256 MB, and an optional display circuit card supporting OpenGL and having 4 MB of memory. A recommended configuration for better performance would include, for example, a “Pentium III” processor at 500 MHz or faster, memory of 256 MB or greater, disk space of 500 MB or more, swap space of 500 MB or more, and an optional display card having 8 MB of memory or more, supporting resolution of 1024×768.

Preferably, the software used in the computing system described above includes, for the server machine, operating system software such as “Windows NT Server 4.0” from Microsoft Corporation, with Service Pack 5, Version 1280 (10 Jun. 1999) or more recent, with database management server software such as “Oracle Server Standard Edition 8.1” from Oracle Corporation, or better. The software used in a preferred embodiment of the user machine includes operating system software such as “Windows NT Workstation 4.0” from Microsoft Corporation, with Service Pack 5, version 1280 (10 Jun. 1999) or more recent, as well as “Oracle Client Standard Edition Version 8.1” or better. The client machine will also be compliant with the “Java” programming language (Java Runtime Environment 1.2.2). As will be known to those skilled in the art, other configurations may be suitable, depending on the applications being used and the computer performance desired.

Database Interface

The database interface was a Java-based interface with useful features. The database is interfaced to a molecular graphics package that includes 3-D visualization, including wire-frame representations; secondary structure ribbons; and solid surfaces, and structure analysis tools. The database also provides an interface to access all of the collected files from the same 3-D structure. The database interface also provides access to other databases, such as databases of chemical structures and public domain databases such as GenBank and the Protein Data Bank. The OpenGL and C++ module has real-time interaction with the sequence display and sequence analysis modules, such that highlighting residues in one display results in highlighting those same residues in other displays.

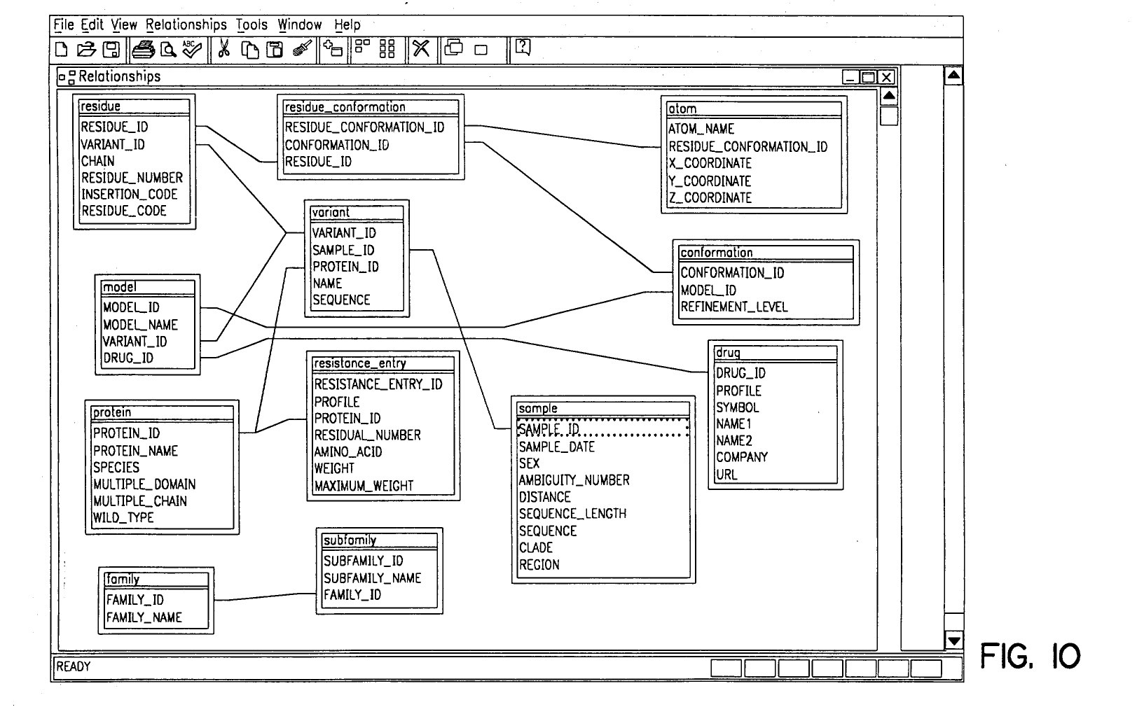

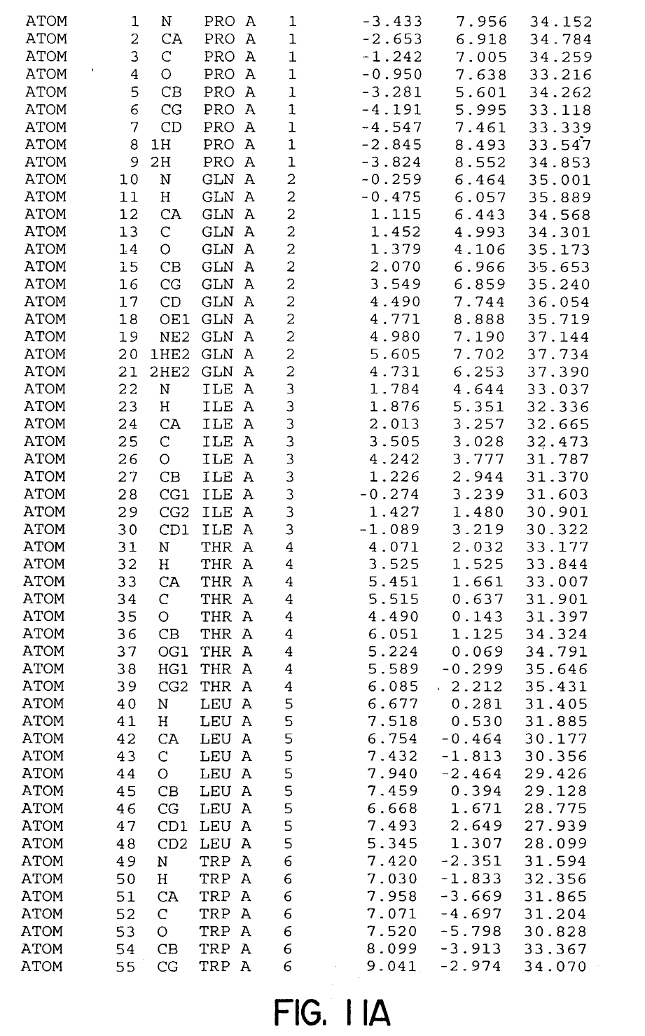

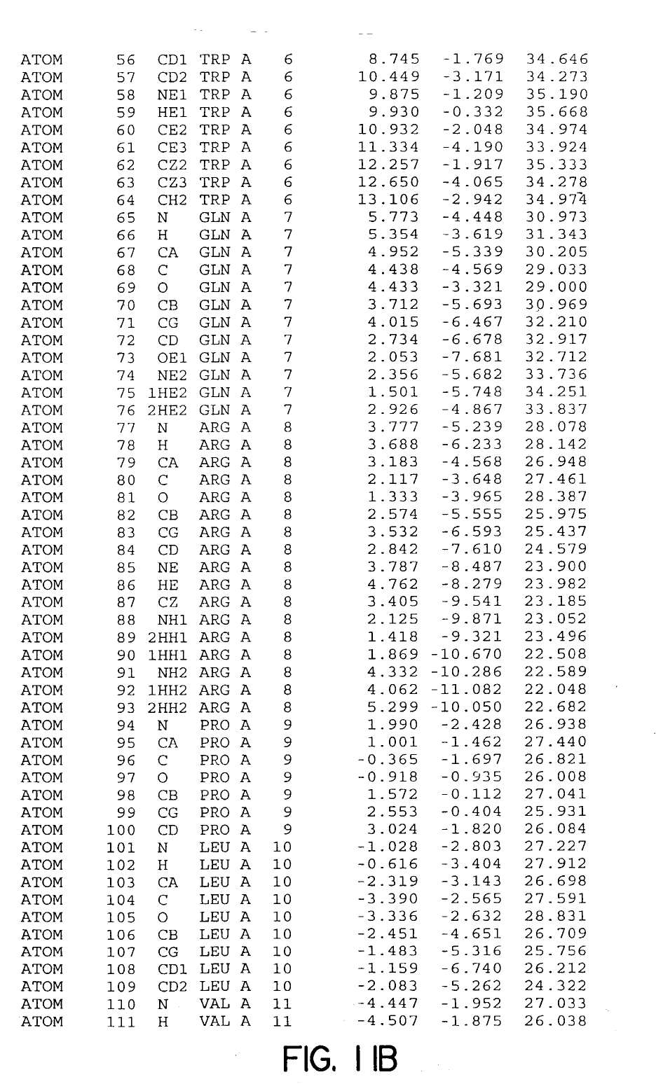

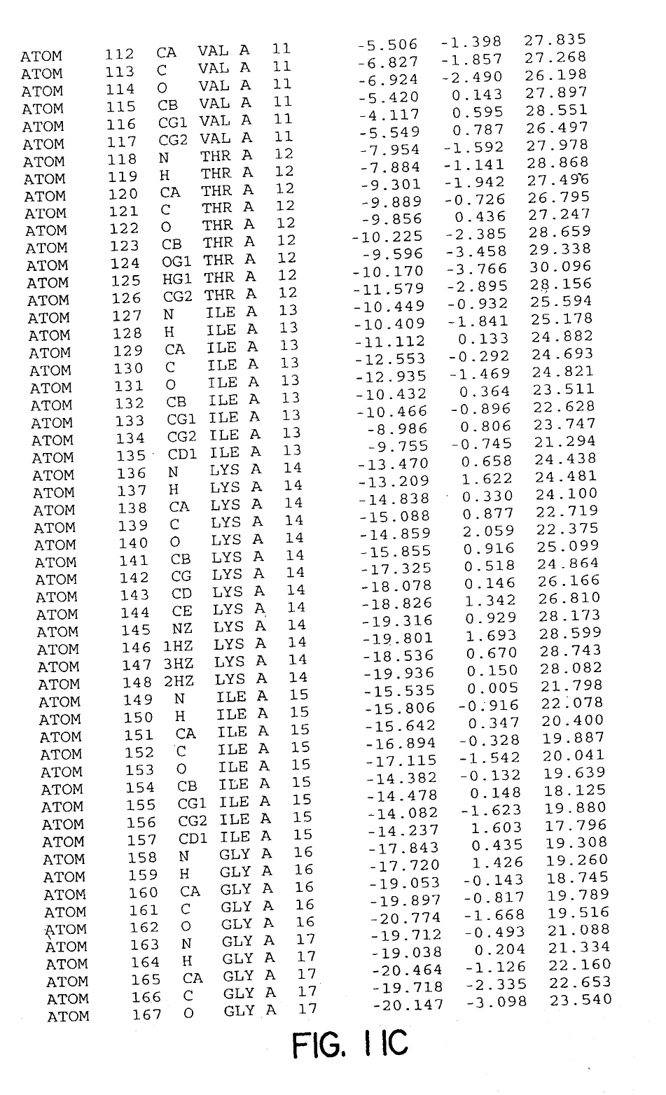

The relational database containing the protein information may be structured according to relational objects to facilitate the analysis and computation processes described in the preceding examples. FIG. 10 is a graphical representation of the database objects for the system described herein. The database is organized by classes, each of which is characterized by data attributes and subclasses for the proteins.

FIG. 10 shows that the database design includes classes comprising Variant and related classes of Sample, Residue, Model, Resistance_Entry, and Protein. Other classes include Conformation, Residue_Conformation, Atom, Drug, Family, and Subfamily. These classes store attribute data values and specify class parameters and behaviors to provide the functionality described herein.

For example, FIG. 10 shows that the Variant class stores parameters to specify a variant, including subclasses that specify a Variant_ID, Sample_ID, Protein_ID, Name, and Sequence, where Variant_ID is the identification number of the variant; Sample_ID is the identification number of the sample from which HIV PR and RT were obtained; Protein_ID is the identification number of the protein i.e. PR or RT; Name is the name of the variant distinguishing it from other variants encoded by the same DNA due to ambiguities in the nucleic acid sequence; and Sequence is the nucleotide or amino acid sequence. Similarly, FIG. 10 shows that the Sample class includes subclasses relating to a specific sample and which specify Sample_ID, Sample_Date, Sex, Ambiguity_Number, Distance, Sequence_Length, Sequence, Clade, and Region, where Sample_ID is as defined herein; Sample_Date is the date the sample was obtained; Sex is the gender of the sample donor; Ambiguity_Number is fraction of ambiguous nucleotide positions; Distance is a normalized number the variation of an amino acid from the master lade; Sequence_Length is the length of the sequence; Sequence is as defined herein; Clade is the master sequence; and Region is the geographic location from which the sample was obtained. The Model class includes subclasses comprising Model_ID, Model_Name, Variant_ID, and Drug_ID, where Model_ID is the identification number of the 3-D protein model; Model_Name is the name of the 3-D protein model; Variant_ID is as defined herein; and Drug_ID is the identification number of the drug i.e. antiviral drug. The atom class includes the subclasses comprising Atom_Name, Residue_Conformation_ID, X_Coordinate, Y_Coordinate, and Z_Coordinate, where Atom_Name is the name of atom in the 3-D protein structure; Residue_Conformation_ID is the identification number of the amino acid conformation in a 3-D structure; and X_Coordinate, Y_Coordinate, and Z_Coordinate are the coordinates of the 3-D protein structure. The conformation class includes the subclasses comprising Conformation_ID, Model_ID, and Refinement_Level, where Conformation_ID is the identification number of a conformation of a 3-D structure; Model_ID is as defined herein, and Refinement_Level is the number of times the conformation was refined energetically. The drug class includes the subclasses comprising Drug_ID, Profile, Symbol, Name1, Name2, Company, and URL, where Drug_ID is as defined herein; Symbol is the FDA symbol for the drug; Name1 is the name of the drug, Name2 is an alternative name of the drug; Company is the company that makes the drug; and URL is the website address of the company that makes the drug. The residue_conformation class includes the subclasses comprising Residue_Conformation_ID, Conformation_ID, and Residue_ID, where Residue_Conformation_ID is as defined herein; Conformation_ID is as defined herein; and Residue_ID is the identification number of the amino acid. The Resistance_Entry class includes the subclasses comprising Resistance_Entry_ID, Profile, Protein_ID, Residual_Number, Amino_Acid, Weight, and Maximum_Weight, where Protein_ID is as defined herein, Amino_Acid is the amino acid. The Family class includes the subclasses comprising Family_ID and Family_Name, where Family_ID is the identification number of the protein family and Family_Name is the name of the protein family. The SubFamily class includes the subclasses comprising SubFamily_ID, SubFamily_Name, and Family_ID, where SubFamily_ID is the identification number of the protein subfamily, SubFamily_Name is the name of the protein subfamily, and Family_ID is as defined herein. The Protein class includes the subclasses comprising Protein_ID, Protein_Name, Species, Multiple_Domain, Multiple_Chain, and Wild_Type, where Protein_ID is as defined herein, Protein_Name is the name of the protein i.e. RT or PR; Species is the species of the source of the protein i.e. humans; Multiple_Domain is the domain of the protein i.e. p66 or p51 in the case of RT; Multiple_Chain is the a or b chain in the dimers of RT and PR; and Wild_Type is the wild-type protein sequence for RT and PR. The residue class includes the subclasses comprising Residue_ID, Variant_ID, Residue_Number, Insertion_Code, and Residue_Code, where Residue_ID is the identification number of the amino acid, Variant_ID is as defined herein, Chain, Residue_Number is the numbering of an amino acid in a protein sequence, Insertion_Code is the identification number if different insertions occur in the amino acid sequence, and Residue_Code is the single letter or 3-letter code of an amino acid. Those skilled in the art will understand the database design exemplified in FIG. 10. It should be understood that other classes or parameters may be included, as selected by those skilled in the art, for the desired database design.

Database Content

The databases contain information on the variants of HIV PR and RT present in patient populations. The master amino acid sequence, nucleic acid sequence, and 3-D structure are obtained from GenBank; an exemplary master sequence is set forth in SEQ ID No. 118. Nucleotide sequences exhibiting polymorphisms and the corresponding structural variant protein sequences are determined by isolating nucleic from viruses and viral nucleic acid obtained from the blood samples of patients throughout the US, as well as from other countries, using sequencing methods well known in the art. The sequences were inputted into the RT and PR databases. Exemplary of the nucleotide sequences and the encoded amino acids for HIV RT and PR in this data base are set forth in SEQ ID NOS. 3 to 117, where r is g or a; y is t/u or c; m is a or c; k is g or t/u; s is g or c; w is a or t/u; b is g or c or t/u; d is a or g or t/u; h is a or c or t/u; v is a or g or c; and n is a or g or c or t/u or unknown or other. The amino acid sequences of the wild type and structural variants are used to create 3-D protein structures which are deposited into the databases.

1. 3-D Protein Models

The structure of the wild-type or master sequence model of PR and RT were obtained from the crystal structures found in PDB. The initial structure was refined energetically using BPMC with an ECEPP force field as described in Example 1. The quality of the model was assessed by calculating Normalized Residue Energies (NREs), where models with eₐᵥ23 1.5 require further energetic refinement; and models with eₐᵥ<1.5 were deposited into the database as described herein. The 3-D protein structures of the variant sequences were generated by comparing these structures to the master sequence (see, e.g., SEQ ID No. 118; i.e., homology modeling) and energetically refining the models ab initio, using the same force field and BPMC procedure as the master sequence and applying the same quality control standard as described herein. FIG. 11 is a tabulation of the 3-D coordinates of an exemplary HIV PR entry in a database that includes 3-D structures. For US purposes and where permitted, Tables 4 and 5 are provided electronically on CD ROM. These Tables house the coordinates that represent the 3-D protein structures of proteins encoded by the nucleic acids set forth in SEQ. ID. NOS. 3-117. It will be noted that these sequences encode a full length PR and about 200 nucleotides the p51 subunit, which is the subunit of interest herein. To construct the full-length 3-D structure, the 3-D structure of each encoded portion of the p51 subunit was generated and then combined with the structure of the master sequence to produce a full-length structure.

These 3-D structures in the database can be selected and exported into computational docking programs for analyzing protein-drug interactions on known drugs, new drugs or modified drugs. The database can be mined to find protein models that correspond to patients with a particular genetic polymorphism, patients with the most commonly occurring polymorphism, to a relevant patient subpopulation (e.g., gender, age, race, or other characteristic), to patients receiving a specific treatment regimen, to patients exhibiting a particular clinical response, to structural invariants, or to other relevant criteria. Drugs can be docked into the active sites of PR and RT and subsequently energetically refined using an ECEPP force field and BPMC as described in Example 1. The quality control is that the protein-drug complex represents a low energy conformation, which may take several iterative BMPC cycles. Then, the binding energies of the protein-drug complexes can be estimated using the methods of Example 1. Drug designers can modify the structures of drugs or design new drugs, using methods well known in the arts, to maximize the drug binding to the models generated by this database.

2. Other Data

Each PR or RT nucleotide sequence in the database has associated with it an identification number, the nucleotide sequence length, the translated amino acid sequence (or sequences in cases of ambiguous nucleotide positions), a 3-D structure for each amino acid sequence (from which a number of structurally related values are calculated), the genotyping date, the gender of the patient, the geographical location from which the sample was sent, the lade of the sequence, the fraction of ambiguous nucleotide positions, drug information, and other clinical information.

Database Usage

A query menu allows the user to retrieve data based on the various fields: sample ID, residue number (with or without specific amino acid mutation), date gender, geographic location, distance from the master sequence, and other useful queries. The set of sequences that satisfies the user's query are brought up in a sequence display module, which have variations from the master sequence indicated initially, although the sequences can be highlighted according to predicted resistance. This subset of sequences can be subjected to further analyses. For example, a histogram summarizing the number of mutations at each position in the subset can be generated. The 3-D structures for any of the variants in the database can be displayed and analyzed in the structure visualization module, allowing the user to compare the similarities and differences between 3-D structures by superimposing the 3-D structures. The user and also export these structures into programs for protein-binding studies as described herein. Thus, by mining the databases, a user will access 3-D structures and clinical and sample information that can be used in and correlated with protein-drug binding studies of HIV PR and RT.

Database Applications

The HIV PR and RT databases have many applications. The applications include, but are not limited to, any application and method provided herein, such as databases that assist in de novo drug design and drug binding calculations. In particular, the database can be used in the design of 2nd and 3rd generation drugs to combat potential resistance to HIV therapy, and it can be used in the design of drugs that will impact a broad spectrum of the infected population. The databases provide the ability to design drugs that focus on the most highly conserved regions of a drug target and drugs that will avoid resistance to mutation. The database could be used to rank drug candidates by likely efficacy within a given subpopulation of patients (e.g. age, race, gender) in pre-clinical trials and to predict the most effective drug regimen to give a patient, and for designing clinical trials.

Since modifications will be apparent to those of skill in this art, it is intended that this invention be limited only by the scope of the appended claims.

What is claimed is:

1. A computer-based method of selecting drug therapies for subjects based on genetic polymorphisms, comprising:
obtaining amino acid sequences of a target protein that is the product of a gene exhibiting genetic polymorphisms, wherein the sequences represent different genetic polymorphisms;
generating 3-D protein structural variant models from the sequences;
computationally docking drug molecules with the target protein models;
energetically refining the docked complexes;
determining the binding interactions between the drug or potential new drug candidate molecules and the models; and
selecting drug therapies based on the drug or drugs that have the most favorable binding interactions with the structural variant models.

2. The method of claim 1, wherein the binding interactions are determined by:
calculating the free energy of binding between the protein structural variant and the docked drug molecule; and
decomposing the total free energy of binding based on the interacting residues in the protein active site.

3. A computer-based method for predicting clinical responses in subjects based on genetic polymorphisms, comprising:
obtaining one or more amino acid sequences for a target protein that is the product of a gene exhibiting genetic polymorphisms;
generating 3-D protein structural variant models from the sequences;
building a relational database of protein structural variants derived based on genetic polymorphisms and observed clinical data associated with particular polymorphisms exhibited in the subjects, wherein the database comprises:
3-D molecular coordinates for the structural variant models;
a molecular graphics interface for 3-D molecular structure visualization;
computer functionality for protein sequence and structural analysis;
database searching tools; and
observed clinical data associated with the genetic polymorphisms, subject medical history and subject history associated with the genetic polymorphisms;
obtaining a target protein structural variant based on the same gene associated with a polymorphism in a subject;
generating a 3-D protein model based on the subject's gene sequence;
screening/comparing the 3-D model derived from the subject to the structures contained in the database by:
identifying structures in the database that are similar to the model derived from the subject; and
predicting a clinical outcome for the subject based on the clinical data associated with the identified structures.

4. A computer-based method for designing therapeutic agents that are active against biological targets that have become drug resistant due to genetic mutations, comprising:
obtaining a first 3-D protein structural variant model of a target protein against which a given drug has biological activity;
generating a second 3-D protein structural variant model of the target in which genetic mutations have occurred and against which the same drug is no longer biologically active;
comparing the structures of the first and second model to identify structural differences; and
performing structure-based drug design calculations in order to identify new drugs or modifications to the existing drug to bring about biological activity against the second model.

5. A computer-based method for identifying compensatory mutations in a target protein, comprising:
obtaining the amino acid sequence of a target protein containing multiple amino acid mutations that is expressed in a subject, wherein the structure of a form of the target protein that responds to a particular drug, including the active site, has been structurally characterized;
generating a 3-D structural model of the mutated protein;
comparing the structure of the mutated protein with the form of the protein that responds to the drug to identify structural differences and/or similarities arising from the mutations;
comparing the biological activities of the drug against both the mutated protein and the form of the protein that responds to the drug to determine the effects of the mutations on drug response; and
identifying the mutations in the protein that affect biological activity based on the comparisons.

6. A method for creating a 3-D structural polymorphism relational database, comprising:
obtaining one or more amino acid sequences of a target protein that is the product of a gene exhibiting a genetic polymorphism, wherein sequences represent different genetic polymorphisms;
generating 3-D protein structural variant models from the sequences;
energetically refining the models;
evaluating the quality of the models;
optionally obtaining associated clinical properties or data; and
inputting the model and any associated properties and/or data into a relational database.

7. The method of claim 6, wherein after energetically refining the models, the models are further refined.

8. The method of claim 6, wherein the database comprises amino sequences of two or more polymorphic variants.

9. The method of claim 6, wherein the database comprises amino sequences of ten or more polymorphic variants.

10. The method of claim 6, wherein the database comprises amino sequences of about 100 or more polymorphic variants.

11. The method of claim 6, wherein the database comprises amino sequences of about 1000 or more polymorphic variants.

12. The method of claim 6, wherein the database comprises amino sequences of more than 8000 polymorphic variants.

13. A database created by the method of claim 6.

14. The database of claim 13, comprising variant 3-dimensional structures of a selected target.

15. The database of claim 13 that comprises structures of proteases or polymerases.

16. The database of claim 13, wherein the proteases are viral proteases or polymerases.

17. The database of claim 13, wherein the viral proteases are human immunodeficiency virus proteases and the polymerase is a viral reverse transcriptase.

18. The method of claim 6, wherein quality is assessed by computing the normalized residue energies such that if eₐᵥ is≧1.5 a model is further refined until eₐᵥ is <1.5; if eₐᵥ is <1.5 a model is deposited into the database.

19. A computer system, comprising a database containing data representative of the three dimensional structure of polymorphic variants of a drug target.

20. The system of claim 19, wherein the target is a cell surface receptor or an enzyme.

21. The system of claim 19, wherein the enzyme is a protease or a polymerase.

22. A database, comprising:
sequences of nucleotides encoding a protein or portions thereof, wherein proteins comprise polymorphic variants; and the portions encode a domain of the protein that comprises a site in the protein that binds to a drug candidate; and
the coordinates of 3-dimensional (3-D) structures of the encoded proteins or portions thereof.

23. The database of claim 22 that is a relational database.

24. The database of claim 22 that comprises at least 2 polymorphic variants and the corresponding 3-D structures.

25. The database of claim 24 that comprises more than 10, more than 100, more than 1000, more than 8000, or more than 10,000 polymorphic variants and the corresponding 3-D structures.

26. The database of claim 22, wherein the protein is a receptor or enzyme from a eukaryotic or prokaryotic organism.

27. The database of claim 22, wherein the organism is a pathogen or a mammal.

28. The database of claim 22, wherein the organism is a pathogen is a virus or bacterium and the mammal is a human.

29. The database of claim 22, wherein the protein is a protease or a reverse transcriptase.

30. A database, comprising the sequences of nucleotides set forth in SEQ ID Nos. 3-117 that encode HIV protease or the portion of HIV reverse transcriptase set forth in each SEQ ID.

31. The database of claim 22, further comprising 3-D structural coordinates for a protein or portion thereof comprising sequences of amino acids encoded by each of SEQ ID Nos. 3-117.

32. The database of claim 23, wherein the protein is HIV protease.

33. The database of claim 23, wherein the protein is HIV reverse transcriptase.

34. A method for predicting clinical responses in subjects based on a genetic polymorphism, comprising:
comparing a three-dimensional (3-D) model of a structure of a protein from a biological sample from a subject to 3-D structures contained in a database;
identifying a structure in the database that is similar to the model of a protein from the subject; and
predicting a clinical outcome for the subject based on the clinical data associated with the identified structures.

35. The method of claim 34, wherein:
the clinical outcome is the efficacy of a treatment; and
the efficacy of a particular treatment for subjects who have the protein in the database is known.

36. The method of claim 34, wherein the protein is an human immunodeficiency virus (HIV) protein.

37. The method of claim 36, wherein the HIV protein is a protease or reverse transcriptase.

38. The method of claim 34, wherein the primary sequence of the target protein is deduced from the sequence of a gene obtained from a subject sample.

39. The method of claim 34, wherein the subject is a human.

40. The method of claim 34, wherein the protein is encoded by gene that has a plurality of polymorphisms.

41. A method for predicting resistance or susceptibility to a particular drug therapy, comprising:
generating a 3-D structural model of a target protein from a subject;
performing protein-drug binding analyses in silico; and
predicting drug sensitivity or resistance based on the protein-drug binding analyses.

42. The method of claim 41, wherein the protein is an HIV protein.

43. The method of claim 42, wherein the HIV protein is a protease or reverse transcriptase.

44. The method of claim 41, wherein the primary sequence of the target protein is deduced from the sequence of a gene obtained from a subject sample.

45. The method of claim 41, wherein the subject is a human.

46. A method for predicting clinical responses in subjects based on genetic polymorphisms, comprising:
comparing a 3-D model of the structure of a protein from biological sample from a subject to 3-D structures contained in a database of claim 38;
identifying structures in the database that are similar to the model of the protein from the subject; and
predicting a clinical outcome for the subject based on the clinical data associated with the identified structures.

47. The method of claim 46, wherein the subject is a human.

48. A computer-based method of selecting drug therapies for subjects based on a genetic polymorphism, comprising:
determining the amino acid sequence of a protein from a biological sample from a subject;
identifying a structural variant model with the same amino acid sequence or similar 3-dimensional (database) structure in a database of three-dimensional structural variants;
selecting a drug therapy for the subject based on the drug or drugs that have the most favorable binding interactions with the structural variant model that corresponds to the protein in the subject sample.

49. The method of claim 47, wherein the protein is encoded by gene that has a plurality of polymorphisms.

50. A computer-based method of selecting drug therapies for subjects based on a genetic polymorphism, comprising:
determining the amino acid sequence of a protein from a biological sample from a subject;
identifying a structural variant model with the same amino acid sequence or the same or similar 3-dimensional (3-D) structure in a database of claim 13;
selecting a drug therapy for the subject based on the drug or drugs that have the most favorable binding interactions with the structural variant model that corresponds to the protein in the subject sample.

51. The method of claim 50, wherein the subject is a human.

52. The method of claim 50, wherein the protein is encoded by gene that has a plurality of polymorphisms.

53. The method of claim 1, wherein a subject is a human.

54. The method of claim 3, wherein a subject is a human.

55. The method of claim 5, wherein a subject is a human.

56. The method of claim 48, wherein the subject is a human.
